(19) United States
(12) Patent Application Publication
(10) Pub. No.: US 2003/0026831 A1
(43) Pub. Date: Feb. 6, 2003

(54) Anionic liposomes for delivery of bioactive agents

(76) Inventors: Aparna Lakkaraju, Minneapolis, MN (US); Janet M. Dubinsky, St. Paul, MN (US); Walter Low, Shorewood, MN (US); Yueh-Erh Rahman, LaJolla, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/131,786

(22) Filed: Apr. 22, 2002

Related U.S. Application Data
(60) Provisional application No. 60/285,337, filed on Apr. 20, 2001.

(51) Int. Cl. A61K 9/127

(52) U.S. Cl. 424/450

(57) ABSTRACT

The present invention relates to the delivery of bioactive agents into cells. More specifically, the present invention relates to methods of using anionic liposomes to deliver bioactive agents, including oligonucleotides, plasmid DNA, RNA, proteins, and drugs, to non-dividing cells. The present invention also relates to compositions that include the anionic liposomes.

### CROSS REFERENCE TO RELATED APPLICATIONS

This application claims the benefit of priority to U.S. Provisional Patent Application Serial No. 60/285,337, filed Apr. 20, 2001, of which is herein incorporated by reference in its entirety.

### GOVERNMENT FUNDING

The present invention was made with government support under Grant Nos. 5R01-AG10034-07 and NS39414-01, awarded by the National Institute of Health (NIH). The Government may have certain rights in this invention.

### FIELD OF THE INVENTION

The present invention relates to the delivery of bioactive agents into cells. More specifically, the present invention relates to methods of using anionic liposomes to deliver bioactive agents, including oligonucleotides, plasmid DNA, RNA, proteins, and drugs, to non-dividing cells. The present invention also relates to compositions that include the anionic liposomes.

### BACKGROUND OF THE INVENTION

Because of their polyanionic nature, oligonucleotides and oligonucleotide analogs suffer from poor lipid bilayer permeability. Oligonucleotides are generally taken up by the cell through the largely inefficient pathway of passive diffusion, with only a few molecules actually gaining entry into the cell. Furthermore, oligonucleotides taken up by passive diffusion, including the uncharged methylphosphonates that are internalized to a greater extent than the others, are ultimately degraded within lysosomes. This not only greatly decreases the amount of antisense oligonucleotide available to the cell, but also increases the potential for toxicity stemming from the breakdown products of modified oligonucleotides. Hence there is a need for the development of delivery systems that will (1) enhance cellular uptake of oligonucleotides, (2) rescue them from being chewed up by lysosomal enzymes, and (3) help achieve therapeutically useful oligonucleotide concentrations in the cytoplasm and/or the nucleus (Stein, C. A., Two problems in antisense biotechnology: in vitro delivery and the design of antisense experiments. Biochim. Biophys. Acta., 1489, 45-52 (1999b)).

For oligonucleotides that act via an RNase H-dependent mechanism, nuclear as well as cytoplasmic localization is important, since this enzyme is reported to be located primarily in the nucleus, with a small fraction in the cytoplasm (Hostomsky et al., Ribonucleases H. In Nucleases. S. M. Linn, R. S. Lloyd, and R. J. Roberts, editors. Cold Spring Harbor Laboratory Press. 341-376 (1993)). For oligonucleotides that act via steric hindrance, cytoplamic localization is sufficient, while those that interfere with mRNA splicing need to make their way into the nucleus.

Current Oligonucleotide Delivery Systems

Olignucleotides can be delivered into cells via mechanical, electrical, or chemical methods, as summarized in Table 1. Chemical methods are the most promising for future clinical applications. Liposomes are the most widely used systems for nucleic acid delivery today.

TABLE 1
Methods used to deliver oligonucleotides to cells.

| Oligonucleotide Delivery Method | Vector System |
|---|---|
| Mechanical | Microinjection |
| | Particle Bombardment |
| Electrical | Electroporation |
| Chemical (intracellular delivery) | Liposomes |
| | Cationic Lipids |
| | Cationic polymers |
| | Nanoparticles |
| | Proteins |
| Chemical (membrane permeabilization) | Streptolysin O |
| | Amphotericin B |

References: Akhtar et al., 2000; Bally et al., 1999; Garcia-Chaumont et al., 2000; Luo and Saltzman, 2000.

Cationic Lipids and Liposomes

Cationic liposomes, composed of positively charged lipids, complexed to oligonucleotides via electrostatic interactions, are commonly used to deliver oligonucleotides in vitro (Capaccioli et al., Cationic lipids improve antisense oligonucleotide uptake and prevent degradation in cultured cells and in human serum. Biochem. Biophys. Res. Commun., 197, 818-25 (1993)). Due to their positive charge, these complexes have a high affinity for negatively charged cell membranes and are thought to enter the cells via adsorptive endoctytosis. Many commercially available cationic liposomes have a “helper” lipid, dioleoyl phosphatidyl ethanolamine (DOPE) (Harvie et al., Characterization of lipid DNA interactions. I. Destabilization of bound lipids and DNA dissociation. Biophys. J., 75, 1040-51 (1998); Hope et al., Cationic lipids, phosphatidylethanolamine and the interacellular delivery of polymeric, nucleic acid-based drugs. Mol. Membr. Biol., 15, 1-14 (1998)). This lipid forms non-bilayer hexagonal structures at the low pH found in the endosomal compartments and destabilizes the endosomal membrane to release oligonucleotides into the cytoplasm (Marcusson et al., Phosphorothioate oligodeoxyribonucleotides dissociate from cationic lipids before entering the nucleus. Nucleic Acids. Research., 26, 2016-2023 (1998); Zelphati and Szoka, Intracellular distribution and mechanism of delivery of oligonucleotides mediated by cationic lipids. Pharmaceutical Research. 13, 1367-1372 (1996)). The efficacy of these cationic liposome-based delivery systems is dependent on the cationic lipid, cell type, presence of serum, oligonucleotide chemistry, and the method of complex preparation. Biodegradable nanoparticles, composed of poly(isohexylcyanoacrylate) and quaternary ammonium salts, were reported to protect oligonucleotides from nucleases and efficiently deliver them to human macrophage cell lines (Chavany et al., Polyalkylcyanoacrylate nanoparticles as polymeric carriers for antisense oligonucleotides. Pharm. Res., 9, 441-9 (1992); Chavany et al., Adsorption of oligonucleotides onto polyisohexylcyanoacrylate nanoparticles protects them against nucleases and increases their cellular uptake. Pharm. Res., 11, 1370-8 (1994)). A major limitation of these particulate systems is the toxicity associated with quaternary ammonium salts and the tendency of the particles to dissociate in the presence of physiological salt concentrations (Lambert et al., Effect of polyisobutylcyanoacrylate nanoparticles and lipofectin loaded with oligonucleotides on cell viability and PKC alpha neosynthesis in HepG2 cells. Biochimie., 80, 969-76 (1998)).

Cationic polymers such as polyamidoamine (PAMAM) dendrimers and polyethyleneimine (PEI) have been studied extensively for oligonucleotide delivery via electrostatic complexation (Tang and Szoka, The influence of polymer structure on the interactions of cationic polymers with DNA and morphology of the resulting complexes. Gene Ther., 4, 823-32 (1997)). PAMAM dendrimers are highly branched, spheroidal polycationic polymers that can be synthesized to have a specific number of amines per dendrimer and a specific diameter. Although oligonucletides delivered by dendrimers are reported to down-regulate target protein to 35-40% of control levels (Hughes et al., Evaluation of adjuvants that enhance the effectiveness of antisense oligodeoxynucleotides. Pharmaceutical Research, 13, 404-410 (1996)), efficient intracellular delivery depends on cellular mitotic activity (Helin et al., Cell cycle-dependent distribution and specific inhibitory effect of vectorized antisense oligonucleotides in cell culture. Biochem. Pharmacol., 58, 95-107 (1999)).

A synthetic cationic lipid, dioleoyl propyl trimethylammonium (DOTMA or Lipofectin) that could form complexes with DNA and facilitate delivery of DNA into cells was reported in 1989 (Felgner and Ringold, Cationic liposome-mediated transfection. Nature, 337, 387-8 (1989)). Transfection of mouse fibroblasts by mixtures of DOTMA and DOPE complexed to the chloramphenical actetyl transferase (CAT) plasmid resulted in 300 plasmid copies/cell compared to 10 copies/cell when delivered by the traditional method of calcium phosphate precipitation.

To reduce the cytotoxicity of DOTMA, metabolizable quaternary ammonium salts such as dioleoyl trimethyl ammonium propane (DOTAP) and dimethylaminoethane carbamoyl cholesterol (DC-Chol), with comparable transfection efficiencies to DOTMA but reduced toxicity, were synthesized. Nevertheless, the caveats for successful transfection by cationic lipids that were listed in the Felgner reference more than a decade ago still hold true. These limitations include a requirement for serum-free medium, variability of optimal cationic lipid/DNA ratio, and marked cell-type dependence of transfection efficiency; and they apply to polycationic polymers as well as cationic lipids. Most importantly, although cationic lipids have been used extensively in cell lines, they have been unsuccessful for widespread delivery of oligonucleotides to primary cells such as neurons (Ajmani and Hughes, 3Beta [N-(N′,N′-dimethylaminoethane)-carbamoyl] cholesterol (DC-Chol)-mediated gene delivery to primary rat neurons: characterization and mechanism. Neurochem. Res., 24 699-703 (1999); Ajmani et al., Enhanced transgene expression in rat brain cell cultures with a disulfide-containing cationic lipid. Neurosci. Lett., 277, 141-4 (1999)) and lymphocytes (Bennet, Use of cationic lipid complexes for antisense oligonucleotide delivery. In Applied Antisense Oligonucleotide Technology. C. A. Stein and A. M. Krieg, editors. Wiley-Liss, New York, 129-145 (1998)). Recent studies have shown that uptake of cationic lipid-oligonucleotide complexes requires mitotic activity, and that the complexes are best taken up in the G1/S phase of the cell cycle (Mortimer et al., Cationic lipid-mediated transfection of cells in culture requires mitotic activity. Gene Therapy, 6, 403-411 (1999)). This may explain in part the cell-type dependence of their effect. In addition, many of these cationic lipids demonstrate unacceptable levels of toxicity when used both in vitro and in vivo (Luo and Saltzman, Synthetic DNA delivery systems. Nat. Biotechnol., 18, 33-37 (2000)). The use of cationic lipids for delivering oligonucleotides in vivo is further complicated by the ability of these synthetic lipids to cause immune reactions and become inactivated by serum components. The cell-type dependence of both delivery efficiency and toxicity require strict optimization of the lipid-DNA charge ratio for each application.

Physicochemical and morphological characterization of the cationic lipid (or polymer) complexes with nucleic acids revealed significant polydispersity in size and an array of shapes (Jaaskelainen et al., Oligonucleotide-cationic liposome interactions. A physicochemical study. Biochim. Biophys. Acta., 1195, 115-23 (1994); Labat-Moleur et al., An electron microscopy study into the mechanism of gene transfer with lipopolyamines. Gene Ther., 3, 1010-7 (1996); Sternberg et al., New structures in complex formation between DNA and cationic liposomes visualized by freeze-fracture electron microscopy. FEBS Lett., 356, 361-6 (1994)). These complexes are highly unstable at physiological salt concentrations, often forming aggregates that are several microns in size. It is apparent that a better understanding of the physiocochemical properties and structures of lipid-based oligonucleotide delivery systems is necessary for the development of the ideal “one size fits all” vector.

Anionic Liposomes

Prior to the discovery that cationic lipids could deliver nucleic acids to cells in vitro, a few studies reported transfection of cells with DNA encapsulated in liposomes composed of the anionic lipid phosphatidylserine and cholesterol. Using these anionic liposomes, Simian Virus 40 (SV 40) DNA was delivered to monkey kidney cells (Fraley et al., Introduction of liposome-encapsulated SV40 DNA into cells. J. Biol. Chem., 255, 10431-5 (1980)) and the thymidine kinase gene to mouse L cells (Schaeffer-Ridder et al., Liposomes as gene carriers: efficient transformation of mouse L cells by thymidine knase gene. Science, 215, 166-168 (1981)) with efficiencies of 0.1% and 10%, respectively. Oligonucleotides were “encapsulated” into phosphatidylserine liposomes in the presence of 10 mM CaCl₂, which resulted in the formation of cochleate bodies (Burch and Mahan, Oligonucleotides antisense to the interleukin 1 receptor mRNA block the effects of interleukin 1 in cultured murine and human fibroblasts and in mice. J. Clin. Invest., 88, 1190-6 (1991); Loke et al., Deliveyr of c-myc antisense phosphorothioate oligodeoxynucleotides to hematopoietic cells in culture by liposome fusion: specific reduction in c-myc protein expression correlates with inhibition of cell growth and DNA synthesis. Curr. Top Microbiol. Immunol., 141 282-9 (1988)). However, intracellular delivery of oligonucleotides (ONs) in these studies required polyethylene glycol-induced fusion between liposomes and the plasma membrane, seriously limiting further application of these liposomes.

Anionic liposomes prepared using conventional techniques were initially investigated for oligonucleotide delivery, but were soon abandoned due to poor encapsulation of the oligonucleotides within the aqueous compartment of the liposomes (Akhtar et al., Interactions of antisense DNA oligonucleotide analogs with phospholipid membranes. Nucleic Acids Res., 19, 5551-5559 (1991a); Hughes et al., Evaluation of adjuvants that enhance the effectivenss of antisense oligodeoxynucleotides. Pharmaceutical Research, 13, 404-410 (1994)).

Glutamate-Mediated Excitotoxicity

Glutamate is the principal excitatory neurotransmitter in the brain. The term “excitotoxicity,” coined by Olney in 1978 (Olney, Neurotoxicity of excitatory amino acids. In Kainic acid as a tool in neurobiology. J. W. Olney and P. L. McGreer, editors. Raven Press, New York, 95-121 (1978)), refers to the excessive stimulation of glutamate recteporns, followed by massive influx of calcium ions into neurons, resulting in neuronal injury. Excitotoxicity is the common underlying mechanism in the etiology of acute pathological conditions like cerebral ischemia and traumatic brain injury and chronic neurodegenerative states such as Alzheimer's disease, Huntington's disease and AIDS-related dementia (Choi, Glutamate neurotoxicity and diseases of the nervous system. Neuron., 1, 623-634 (1988)). The outcome of excitotoxicity in neurodegeneration is the progressive loss of neurons through a cascade of molecular events, cumulatively called programmed cell death or apoptosis or necrosis.

Any depletion of cellular energy stores, as can occur during cerebral ischemia, leads to a reversal of the glutamate reuptake system (due to decreased Na⁺/K⁺ ATPase activity and increased intracellular sodium concentrations) and breakdown of the glutamate-glutamine cycle. The resulting continued presence of glutamate at the synaptic cleft causes incessant activation of the post-synaptic neuron. This sets in motion a vicious cycle of glutamate release, calcium influx, further release of glutamate, further calcium influx, and overstimulation of the post-synaptic neuron ad infinitum.

Calcium is an important intracellular second messenger, and it can lead to apoptosis. The biochemical events that execute the cell death process are highly conserved. In mammalian cells, there are two major pathways, the Fas/Fas ligand pathway and the mitochondrial pathway, which is the more important. Several lines of evidence point to expression of the protein p53 as an activator of the mitochondrial pathway. Acute conditions such as cerebral ischemia have been shown to induce p53 expression in neurons exhibiting morphological features of apoptosis (Li et al., p53-immunoreactive protein and p53 mRNA expression after transient middle cerebral artery occlusion in rats. Stroke, U25, 849-55; discusssion 855-6 (1994); Sakhi et al., p53 induction is associated with neuronal damage in the central nervous system. Proc. Natl. Acad. Sci. U.S.A., 91, 7525-9 (1994); Sakhi et al., Nuclear accumulation of p53 protein following kainic acid-induced seizures. Neuroreport., 7, 493-6 (1996)). Studies showed that neurons in mice lacking the p53 gene were protected from excitotoxicity (Morrison et al., Loss of the p53 tumor suppressor gene protects neurons from kainate-induced cell death. J. Neurosci., 16, 1337-45 (1996)). p53 levels in the cell are regulated primarily at the level of translation and by stabilization of the protein against proteolysis. Following DNA damage, translation of the existing levels of p53 mRNA is increased (Kastan et al, Participation of p53 in the cellular response to DNA damage. Cancer Research, 51, 6304-6311 (1991); Fu and Benchimol, Participation of the human p53 3'UTR in translational repression and activation following gamma-irradiation. Embol. J., 16, 4117-25 (1997)). Thus, one way to protect against apoptosis in neurons would be to reduce p53 production by reducing p53 mRNA translation. One approach to this is to use antisense oligonucleotides that hybridize to the p53 mRNA and prevent expression of the p53 protein.

Selective inhibition of gene expression with antisense oligonucleotides (AsONs) is both a popular technique for probing fundamental questions of neuroscience (Sattler et al., 1999) and a potential therapeutic strategy for treatment of neurodegenerative diseases (Gonzalez-Zulueta et al., 1998). However, the elegance of the antisense concept belies the considerable challenge of their intracellular delivery (Bally et al., 1999). Chemical modifications of ONs that enhance nuclease-resistance (e.g., phosorothioates) give poor cellular uptake (˜5-10%) and cause non-sequence-specific effects, raising questions about the efficacy and selectivity of antisense drugs (Stein, 1999). Cationic lipids and polycationic polymers, used as ON delivery vectors, have met with limited success due to a number of variables that seem to affect vector performance (Bally et al., 1999; Zabner et al., 1995). Mechanistic aspects of cationic lipid-mediated delivery are poorly understood because of the physical hetergeneity of cationic lipid-ON complexes (Jaaskeleainen et al., 1994) that may contribute to their cytotoxicity.

Application of antisense technology to the nervous system presents a greater challenge because of the post-mitotic nature of neurons and their exquisite sensitivity to their microenvironment. Cationic lipids and polymers have been used to deliver nucleic acids to neurons, generally at efficiencies of 0.5-5% (Kaech et al., 1996). Factors that influence transgene expression or target protein inhibition include neuronal maturity at the time of transfection, type of cationic lipid used (Kaech et al., 1996), and the net charge of the lipid-DNA complex (Schwartz et al., 1995). Cationic lipids per se have also been reported to be toxic to neurons (Azzazy et al., 1995; Kaech et al., 1996).

Available data suggest that neurons take up exogenous macromolecules less readily and with slower internalization kinetics compared to nonpolarized or mitotically active cells. For instance, liposomes composed of zwitterionic phospholipids were internalized by only 20% of hippocampal neurons (Kobayashi et al., 1992). The extent of internalization of liposomes composed of synthetic cationic phospholipids was ˜1000-fold lower in cortical neurons compared to neuroblastoma cells (Ajmani et al., 1999). Transport of molecules like the cholera toxin from the plasma membrane to intracellular organelles decreases with neuronal development, from ˜100% in 1-day-old neurons to <10% in 14-day-old neurons (Sofer and Futerman, 1996). Interestingly, even basic fibroblast growth factor and transferrin are endocytosed with slower kinetics in neurons compared to astrocytes (Swaiman and Machen, 1986; Walicke and Baird, 1991). As data on endocytosis in other cell types cannot be directly extrapolated to neurons, further studies on the internalization mechanisms in neurons are important if the delivery of therapeutic proteins and nucleic acids to these post-mitotic cells is to be achieved.

There is a need for new ways to deliver oligonucleotides to non-dividing cells (e.g., neuronal cells), since cationic lipids and cationic liposomes are not useful with non-dividing cells, and neurons do not divide. Furthermore, present techniques of using current anionic liposomal formulations are impractical because they do not encapsulate significant amounts of oligonucleotides and/or require polyethylene glycol-induced fusion of the liposomes with cells for intracellular delivery.

### BRIEF DESCRIPTION OF THE FIGURES

FIG. 1. Micrographs and fluorescence micrographs of neurons treated with anionic liposomes containing oligonucleotides. AL-Cy3ON, Uptake of Cy3-ONs encapsulated in anionic liposomes by neurons. Neurons were incubated with AL-Cy3ONs for 1 h at 37° C. in the presence of serum. Note the punctate fluorescence in the cytoplasm and the diffuse nuclear label. AL-pEGFP, Expression of EGFP in neurons. Neurons were treated for 48 h with 1 μg pEGFP encapsulated in anionic liposomes. No treatment, Untreated control cells. Scale bar: 10 μm.

FIG. 2. Micrographs and fluorescence micrographs of various cell types showing delivery of Cy3-ONs by anionic liposomes to a variety of cell types. Cells were incubated with 1 μM Cy3-ONs in anionic liposomes for 1 h at 37° C., fixed and imaged. Note the punctate cytoplasmic and diffuse nuclear fluorescence. Scale bar: 10 μm.

FIG. 3. Micrographs and fluorescence micrographs of fibroblasts showing cell surface expression of LRP is important for the rapid uptake of AL-Cy3ON. After a 1-h incubation with AL-Cy3ONs, Cy3 label was visible in (a) LRP-expressing MEF-1 cells but not in (b) LRP-deficient PEA-13 cells. Following a 3-h incubation, some Cy3 label was visible in (d) PEA-13 cells, although far less than that seen in (c) MEF-1 cells. Scale bar: 10 μm.

FIG. 4. Micrographs and fluorescence micrographs of neurons showing anionic liposome endocytosis by LRP is independent of HSPG and does not alter neuronal calcium contents. (a) Neurons were treated with 100 μg/ml heparin prior to incubation with AL-Cy3ON. Scale bar: 5 μm. (b) Fura-2 ratios in hippocampal neurons were not altered during perfusion of anionic liposomes labeled with N—Rh-DOPE but increased in response to 100 μM NMDA. Data were from a representative field of 23 neurons from among 110 neurons imaged in 5 experiments. (c) Image taken at 24 min (*) indicated the uptake of N-Rh-DOPE within the same neurons in the field. An image taken at comparable gains and wavelengths prior to the anionic liposome perfusion was blank (not shown).

FIG. 5. Size distribution and factors influencing ON encapsulation in anionic liposomes. A. Representative volume-weighted size distribution of anionic DOPC/DOPG liposomes encapsulating ONs. B. Influence of ionic strength of the hydration medium on the encapsulation efficiency of phosphodiester ONs in anionic liposomes. DOPC/DOPG liposomes were prepared with ONs in 10 mM HEPES buffer containing increasing concentrations of NaCl. *, encapsulation significantly greater than that in buffers with 50 and 150 mM NaCl, p<0.001. C. Anionic charge density and ON chemistry influence encapsulation. *, encapsulation significantly greater than corresponding liposomes containing phosphorothioate ONs, and §, encapsulation significantly greater than 30 and 60 mole % DOPG liposomes containing phosphodiester ONs (p<0.0001, two way ANOVA).

FIG. 6. Photomicrograph of hippocampal neurons. Neurons were incubated for 3 hours with p53 antisense ONs in anionic DOPC/DOPG liposomes prior to glutamate exposure for 48 hours and visualized by differential interference contrast microscopy. Veh, control neurons treated with vehicle alone; glu, 50 μM glutmate. Scale bar: 20 μm.

FIG. 7. Graph of percent neuronal survival with different treatments, showing p53 antisense ONs delivered by anionic DOPC/DOPG liposomes protect glutamate-treated hippocampal neurons from excitotoxic death. Veh, control neurons treated with vehicle alone; glu, 50 μM glutamate; AL-dAs, 1 μM phosphodiester p53 AsONs in anionic liposomes; AL-sAs, 1 μM phosphorothioate p53 AsONs in anionic liposomes; AL-buf, anionic liposomes containaining buffer alone; AL-dScr, 1 μM phosphodiester p53 scrambled ONs in anionic liposomes. *, neuronal survival significantly greater than neurons treated with glu, AL-buffer and AL-dScr, p<0.001. Mean ±S.E.M., n≧9.

FIG. 8. p53 protein levels of neurons. A. Western blot of a p53 immunoprecipitate from a typical experiment. B. Quantitated results are the mean ±S.E.M. of three independent experiments. Veh, control neurons treated wit vehicle; AL-As, 1 μM p53 antisense ONs in anionic liposomes; AL-Ser, 1 μM p53 scrambled ONs in anionic liposomes; glu, 50 μM glutamate. *, p53 expression significantly lower than that in neurons treated with glu alone or AL-Scr and glu, p<0.05.

FIG. 9. Graphs showing the effect of lipid composition and charge on the efficacy and toxicity of the delivery system. A. Comparison of the neuroprotective dose-response curves of p53 antisense ONs encapsulated in DOPC/DOPG (circles) or DOPC/DOPS liposomes (squares) or complexed to cationic DC-Chol/DOPE liposomes in a +/−charge ratio of 1/2 (triangles). Neurons were treated with AsONs for 3 hours prior to glutamate exposure (50 μM, 48 hours). Survival significantly greater than the corresponding AsON dose delivered in DOPC/DOPS liposomes, *, p<0.001 and §, p<0.05. B. DOPS (squares) and DC-Chol/DOPE (triangles), but not DOPG (circles), dose-dependently exacerbate toxicity associated with a sub-maximal concentration (10 μM) of glutamate. Arrowhead: amount of anionic lipid present in liposomes corresponding to a 1 μM final concentration of ON. Arrow: amount of cationic lipid present in complexes corresponding to a +/−charge ratio of 1/2 and 1 μM final concentration of ON. 20, 40, and 100 μg DC-Chol/DOPE correspond to amounts present in complexes of +/−charge ratio 1.6/1, 3.2/1, and 8/1 (μmole lipid/μmole ON), respectively, for a 1 μM final ON concentration. Data expressed as Mean ±S.E.M., n≧9.

FIG. 10. Graph showing percent neuronal survival against glutamate toxicity with various treatments. A, Veh, vehicle; glu, 50 μM glutamate; sMm, 5 μM phosphorothioate ONs with 6 mismatches to the p53 antisense sequence; sScr, 5 μM phosphorothioate p53 scrambled ONs; sAs, phosphorothioate p53 antisense ONs; AL-sAs, phosphorothioate p53 antisense ONs in anionic liposomes. §, for each of the bracketed columns, survival significantly greater than cells treated with glu, sMm, SScr, and 1 μM sAs, p<0.001. Neuroprotection by 5 μM unencapsulated phosphorothioate AsONs is significantly less than a 5 to 10-fold lower concentration delivered by anionic liposomes, *, p<0.01. B. Veh, vehicle; PFT-α, 10 μM Pifithrin-α; glu, 50 μM glutamate; MK, 20 μM MK801; CN, 20 μM CNQX; AL-dAs, 1 μM p53 antisense phosphodiester ONs delivered by anionic DOPC/DOPG liposomes. §, for each of the bracketed columns, survival significantly greater than in neurons treated with glutamate alone, p<0.001. Neuroprotection with AL-dAs greater than with PFT-α, MK-801 or CNQX, *, p<0.05. Data expressed as Mean ±S.E.M. and n≧9.

FIG. 11. Micrographs and fluorescence micrographs of hippocampal neurons after uptake of AL-Cy3ON. Cy3 fluorescence was observed at a, 30 min; b, 1 hr; and c, 3 hrs after incubation at 37° C. Note the strong Cy3 fluorescence in neuronal nuclei in b and c. Internalization, but not binding of AL-Cy3 ON to the plasma membrane, was inhibited at 4° C. (d). Scale bar: 5 μm.

FIG. 12. Incidence of Cy3 fluorescence in the cytoplasm of neurons after various manipulations. A neuron was counted as containing ONs if punctate Cy3 fluorescence was observed in the cytoplasm after 30 min of incubation. Total number of cells imaged per condition ranged from 45 to 120. AL-Cy3ON, anionic liposomes encapsulating Cy3-labeled oligonucleotides; 4° C., incubation performed at 4° C. (in all other cases, incubations were at 37° C.); Suc, 0.45 M sucrose; FK, 1 μM FK506; RAP, 500 nM receptor-associated protein; Hep, 100 μg/ml heparin; Prot, 100 μg/ml protamine sulfate; Noc, 5 μg/ml nocodazole; Wort, 100 nM wortmannin; Cy3ON, neurons incubated with Cy3ONs alone, without liposomes; pCL-Cy3ON, Cy3ONs complexed with cationic liposomes at a net-positive charge; nCL-Cy3ON, Cy3ONs complexed with cationic liposomes at a net-negative charge. In all conditions, the final concentration of Cy3ONs was 2 μM.

FIG. 13. Micrographs and fluorescence micrographs of neurons treated with Cy3ONs in anionic liposomes (AL-Cy3ONs). Pretreatment of neurons with hyperosmolar sucrose (b) or FK506 (c) for 10 min decreased the internalization of AL-Cy3ONs compared to cells treated with AL-Cy3ONs alone for 30 min (a). Scale bar: 5 μm.

FIG. 14. Micrographs and fluorescence micrographs of neurons treated with AL-Cy3ONs. Pretreatment with the LRP antagonist RAP inhibited both binding and endocytosis of AL-Cy3ONs into neurons (b), while treatment with heparin (c) or protamine (d) did not affect liposome endocytosis compared to cells treated with AL-Cy3ONs alone for 30 min (a). Scale bar: 5 μm.

FIG. 15. Micrographs and fluorescence micrographs of neurons treated with AL-Cy3ON. Neurons pretreated with nocodazole (b), cytochalasin D (c) or wortmannin (d) for 10 min prior to AL-Cy3ON incubation exhibited very low levels of Cy3 fluorescence after 30 min compared to cells treated with AL-Cy3ONs alone (a). Scale bar: 5 μm.

FIG. 16. Micrographs and fluorescence micrographs of neurons treated with AL-Cy3ON. To study recycling and degradation of oligonucleotides, neurons were incubated with a, Cy3ONs (red) and OG-Tf (green) for 1 hr; b, Cy3ONs (red) and Alexa488-dextran (green) for 3 hrs; c, Rh-PE (red) and OG-Tf (green) for 1 hr; and d, Rh-PE (red) and Alexa488-dextran (green) for 3 hrs. Areas of colocalization of the probes appear yellow. Scale bar: 5 μm.

FIG. 17. Graph of fluorescence after mixing of N-Rh-PE-labeled liposomes with unlabeled liposomes.

FIG. 18. Micrographs and fluorescence micrographs of neuronal cells. After 30 min of incubation with 2 μM Cy3ONs, a low level of diffuse fluorescence was visible in the neurons (b); cationic lipid-Cy3ON complexes, either with a net-positive charge (c) or with a net-negative charge (d) did not appear to enhance Cy3ON uptake into neurons. On the other hand, 2 μM Cy3ONs encapsulated within anionic liposomes were rapidly internalized by neurons within 30 min of incubation (a).

FIG. 19. Proposed pathway of endocytosis and entracellular traffic of anionic liposomes in hippocampal neurons.

### SUMMARY OF THE INVENTION

The present invention provides a pharmaceutical composition (i.e., an anionic liposomal formulation) wherein an anionic liposome effectively encapsulates a bioactive agent. The anionic liposome can encapsulate more than about 10% of the bioactive agent. Specifically, the anionic liposome can encapsulate about 55% to about 65% of the bioactive agent (e.g., oligonucleotide). The pharmaceutical composition can effectively deliver the bioactive agent to suitable targets. Specifically, the pharmaceutical composition can effectively deliver the bioactive agent (e.g., oligonucleotide), to non-dividing cells. More specifically, the pharmaceutical composition (i.e., anionic liposomal formulation) can include DOPC/DOPG, wherein the ratio of DOPC to DOPG is about 88:12; an antisense oligonucleotide having a sequence of 5′ CTC GAC GCT AGG ATC TGA 3′ (SEQ ID NO:1) targeted to human p53 mRNA or an antisense oligonucleotide having a sequence of 5′ CTG TGA ATC CTC CAT GAC 3′ (SEQ ID NO:2) targeted to rat p53 mRNA; the monovalent cation Na⁺; and the buffer HEPES. Such a pharmaceutical composition can effectively encapsulate about 55% to about 65% of the antisense oligonucleotide and can effectively deliver the antisense oligonucleotide to non-dividing cells.

The present invention provides a pharmaceutical composition. The pharmaceutical composition includes: (a) an anionic liposome; (b) a bioactive agent; and (c) a cation, a buffer, or a combination thereof The anionic liposome includes a phospholipid with a head group. The head group is sn-glycero-phosphocholine; sn-glycero-phospho-rac-(1-glycerol); sn-glycero-phospho-L-serine; sn-glycero-3-phosphate; or a combination thereof. The anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

The present invention also provides another pharmaceutical composition. The pharmaceutical composition includes: (a) an anionic liposome; (b) a bioactive agent; and (c) a cation, a buffer, or a combination thereof. The anionic liposome includes a phospholipid with a head group. The head group is sn-glycero-phosphocholine; sn-glycero-phospho-rac-(1-glycerol); sn-glycero-3-phosphate; or a combination thereof.

The present invention also provides another pharmaceutical composition. The pharmaceutical composition includes: (a) an anionic liposome; (b) a bioactive agent; and (c) a cation, a buffer, or a combination thereof. The bioactive agent is an antisense oligonucleotide having a sequence of SEQ ID NO: 1 targeted to human p53 mRNA or an antisense oligonucleotide having a sequence of SEQ ID NO:2 targeted to rat p53 mRNA.

The present invention also provides a method of delivering a bioactive agent to a target. The method includes contacting the target with a composition, wherein the composition includes: (a) an anionic liposome; (b) a bioactive agent; and (c) a cation, a buffer, or a combination thereof. The anionic liposome includes a phospholipid with a head group. The head group is sn-glycero-phosphocholine; sn-glycero-phospho-rac-(1-glycerol); sn-glycero-phospho-L-serine; sn-glycero-3-phosphate; or a combination thereof. The anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

The present invention also provides a method of delivering a bioactive agent to a target. The method includes contacting the target with a composition, wherein the composition includes: (a) an anionic liposome; (b) a bioactive agent; and (c) a cation, a buffer, or a combination thereof. The anionic liposome includes a phospholipid with a head group. The head group is sn-glycero-phosphocholine; sn-glycero-phospho-rac-(1-glycerol); sn-glycero-3-phosphate; or a combination thereof.

The present invention also provides a method of delivering a bioactive agent to a target. The method includes contacting the target with a composition, wherein the composition includes: (a) an anionic liposome; (b) a bioactive agent; and (c) a cation, a buffer, or a combination thereof. The bioactive agent is an antisense oligonucleotide having a sequence of SEQ ID NO: 1 targeted to human p53 mRNA or an antisense oligonucleotide having a sequence of SEQ ID NO:2 targeted to rat p53 mRNA.

The present invention also provides a method of delivering a bioactive agent to non-dividing cells. The method includes contacting the non-dividing cells with a composition, wherein the composition includes: (a) an anionic liposome; (b) a bioactive agent; and (c) a cation, a buffer, or a combination thereof.

The anionic liposome can be 1,2-dioleoyl-sn-glycero-3-phosphocholine (DOPC); 1,2-dioleoyl-sn-glycero-3-[phospho-rac-(1-gylcerol)] (DOPG); 1,2-dioleoyl-sn-glycero-3-[phospho-L-serine] (DOPS); 1,2-dioleoyl-sn-glycero-3-phosphate (DOPA); or a combination thereof. More specifically, the anionic liposome can be DOPC/DOPG. More specifically, the anionic liposome can be DOPC/DOPG wherein the ratio of DOPC to DOPG is about 88:12.

The mean diameter of the anionic liposome can be about 20 run to about 5 microns. More specifically, the mean diameter of the anionic liposome can be about 75 nm to about 500 nm. More specifically, the mean diameter of the anionic liposome can be about 175 nm to about 225 nm.

The anionic liposome can be present up to about 500 mM in the pharmaceutical composition. More specifically, the anionic liposome can be present in about 2.5 mM to about 30 mM in the pharmaceutical composition. More specifically, the bioactive agent can have a molecular weight of about 250 to about 750 and the anionic liposome can be present in about 5 mM to about 25 mM in the pharmaceutical composition, or the bioactive agent can have a molecular weight of about 750 to about 1500 and the anionic liposome can be present in about 7.5 mM to about 25 mM in the pharmaceutical composition. Alternatively, the bioactive agent can be an oligonucleotide having a length of about 5 bases to about 50 bases and the anionic liposome can be present in about 2.5 mM to about 25 mM in the pharmaceutical composition. Alternatively, the bioactive agent can be an oligonucleotide having a length of about 15 bases to about 30 bases and the anionic liposome can be present in about 2.5 mM to about 25 mM in the pharmaceutical composition. Alternatively, the bioactive agent can be a protein having a molecular weight of up to about 75,000 and the anionic liposome can be present in about 2.5 mM to about 30 mM in the pharmaceutical composition. Alternatively, the bioactive agent can be a protein having a molecular weight of about 1,000 to about 5,000 and the anionic liposome can be present in about 2.5 mM to about 30 mM in the pharmaceutical composition. Alternatively, the bioactive agent can be double or single stranded genetic material, or a fragment thereof having a molecular weight of up to about 1×10⁸ and the anionic liposome can be present in about 2.5 mM to about 40 mM in the pharmaceutical composition. Alternatively, the bioactive agent can be double or single stranded genetic material, or a fragment thereof having a molecular weight of about 1×10⁵ to about 1×10⁷ and the anionic liposome can be present in about 2.5 mM to about 40 mM in the pharmaceutical composition.

The bioactive agent can be an antiviral agent; an antibacterial agent; an antifungal agent; an antineoplastic agent; an anti-inflammatory agent; a radiolabel; a peptide; a protein; an oligonucleotide; RNA; RNAi; ribozymes; transposons; chimeraplasts; a hormone; a carbohydrate; a growth factor; a cytokine; a radiopaque compound; a fluorescent compound; a mydriatic compound; a bronchodilator; a local anesthetic; a nucleic acid sequence; double or single stranded genetic material, or a fragment thereof; an analgesic; an antiparasitic; an antipsychotic; an antispasmodic; an arthritis medication; a biological; a bone metabolism regulator; a calcium channel blocker; a cardiovascular agent; a central nervous system stimulant; a diabetes agent; a diagnostic; a fungal medication; a gastrointestinal agent; a histamine receptor antagonist; an immunosuppressive; a muscle relaxant; a nausea medication; a nucleoside analogue; a parkinsonism drug; a platelet inhibitor; a psychotropic; a respiratory drug; a sedative; a urinary anti-infective; a urinary tract agent; a vitamin; a nucleotide; a signaling molecule; a fluorescent molecule; a bioactive lipid; a neuroactive agent; an energy substrate; or a combination thereof.

The bioactive agent can be an antiviral agent such as acyclovir, zidovudine or the interferons; an antibacterial agent such as an aminoglycoside, a cephalosporin or a tetracycline; an antifungal agent such as a polyene antibiotic, an imidazole or a triazole; an antimetabolic agent such as folic acid, or a purine or a pyrimidine analogue; an antineoplastic agent such as an anthracycline antibiotic or a plant alkaloid; a sterol such as cholesterol; a carbohydrate, e.g., a sugar or a starch; an amino acid, a peptide, an enzyme, an immunoglobulin, an enzyme, a hormone, a neurotransmitter or a glycoprotein; a dye; a radiolabel such as a radioisotope or a radioisotope-labeled compound; a radiopaque compound; a fluorescent compound; a mydriatic compound; a bronchodilator; a local anesthetic; a nucleic acid sequence such as messenger RNA, cDNA, genomic DNA or a plasmid; or a bioactive lipid such as an ether lipid or a ceramide.

More specifically, the bioactive agent can be double or single stranded genetic material, or a fragment thereof. More specifically, the bioactive agent can be a p53 antisense oligonucleotide. More specifically, the bioactive agent can be an antisense oligonucleotide having a sequence of SEQ ID NO:1 targeted to human p53 mRNA or an antisense oligonucleotide having a sequence of SEQ ID NO:2 targeted to rat p53 mRNA.

The bioactive agent can be present up to about 50 mM in the pharmaceutical composition. More specifically, the bioactive agent can be present in about 1 femtoM to about 1 M in the pharmaceutical composition. More specifically, the bioactive agent can be present in about 2 nM to about 10 mM in the pharmaceutical composition. More specifically, the bioactive agent can have a molecular weight of about 250 to about 750 and can be present in about 0.5 mM to about 10 mM in the pharmaceutical composition, or the bioactive agent can have a molecular weight of about 750 to about 1500 and can be present in about 0.5 mM to about 5 mM in the pharmaceutical composition. Alternatively, the bioactive agent can be an oligonucleotide having a length of about 5 bases to about 50 bases and can be present in about 25 μM to about 250 μM in the pharmaceutical composition. Alternatively, the bioactive agent can be an oligonucleotide having a length of about 15 bases to about 30 bases and can be present in about 25 μM to about 250 μM in the pharmaceutical composition. Alternatively, the bioactive agent can be a protein having a molecular weight of up to about 75,000 and can be present in about 25 μM to about 250 μM in the pharmaceutical composition. Alternatively, the bioactive agent can be a protein having a molecular weight of about 1,000 to about 5,000 and can be present in about 25 μM to about 250 μM in the pharmaceutical composition. Alternatively, the bioactive agent can be double or single stranded genetic material, or a fragment thereof, having a molecular weight of up to about 1×10⁸ and can be present in about 2 nM to about 40 nM in the pharmaceutical composition. Alternatively, the bioactive agent can be double or single stranded genetic material, or a fragment thereof having a molecular weight of about 1×10⁵ to about 1×10⁷ and can be present in about 2 nM to about 40 nM in the pharmaceutical composition.

Up to about 100% of the bioactive agent can be encapsulated in the anionic liposome. More specifically, more than about 10% of the bioactive agent can be encapsulated in the anionic liposome. More specifically, more than about 20% of the bioactive agent can be encapsulated in the anionic liposome. More specifically, about 55% to about 60% of the bioactive agent can be encapsulated in the anionic liposome.

The cation can be a monovalent cation. More specifically, the cation can be Na⁺, K⁺, Li⁺, Fr⁺, Rb⁺, or Cs⁺. More specifically, the cation can be Na⁺, K⁺, or Li⁺. The cation can be present up to about 50 nM in the pharmaceutical composition, or up to about 15 nM in the pharmaceutical composition. Specifically, the cation can be present up to about 5 mM in the pharmaceutical composition.

The buffer can maintain the pH of the pharmaceutical composition between about 6.0 to about 8.0. More specifically, the buffer can maintain the pH of the pharmaceutical composition between about 7.0 to about 7.5. The buffer can be present up to about 50 mM in the pharmaceutical composition. More specifically, the buffer can be present up to about 10 mM in the pharmaceutical composition.

The buffer can be HEPES; BES; HEPPS; imidazole; MOPS; TES; TEA; monobasic or dibasic potassium phosphate; monobasic or dibasic sodium phosphate; cacodylic acid; MES; PIPES; glycine amide; glycylglycine; TAPS; boric acid; BIS-TRIS PROPANE; DIPSO; TAPSO; HEPPSO; POPSO; EPPS; TRICINE; BICINE; TAPS; a pharmaceutically acceptable salt thereof; or a combination thereof. More specifically, the buffer can be [4-(2-hydroxyethyl)-l-piperazine ethanesulfonic acid] (HEPES).

The molar ratio of bioactive agent to anionic liposome can be about 10:1 to about 1:1×10¹⁰. More specifically, the molar ratio of bioactive agent to anionic liposome can be about 5:1 to about 1:10,000.

The target can be pleuripotent tissue, e.g., stem cells, embryonic stem cells, or bone marrow-derived stem cells. The target can be a cell of an organ, e.g., brain, central nervous system, peripheral nervous systems, liver, lung, larynx, bone marrow, spleen, kidney, lymphatic system, hematopoetic system, gastric mucosa, small intestine, large intestine, gall bladder, pancreas, salivary gland, teste, ovary, cervix, uterus, muscle, skin, thyroid gland, parathyroid gland, adrenal gland, connective tissue, chondroid tissue, blood vessel, macrophage, pleura, or placenta. The target can be non-dividing cells. The target can be neuronal cells. The target can be hippocampal neuronal cells. The target can be a cell that expresses a receptor belonging to the low-density lipoprotein (LDL) gene family. More specifically, the target can be a cell that possess a low-density lipoprotein receptor-related protein (LRP) receptor. The target can be a cell that possesses an endocytic low-density lipoprotein receptor-related protein receptor. The target can be a cell that possesses a receptor that is expressed in mammalian central nervous system (CNS).

### DETAILED DESCRIPTION OF THE INVENTION

The present invention provides a pharmaceutical composition (i.e., an anionic liposomal formulation) wherein an anionic liposome effectively encapsulates a bioactive agent. The pharmaceutical composition can encapsulate more than about 10% of the bioactive agent. Specifically, the pharmaceutical composition can encapsulate about 55% to about 65% of the bioactive agent (e.g., oligonucleotide). The pharmaceutical composition can effectively deliver the bioactive agent to suitable targets. Specifically, the pharmaceutical composition can effectively deliver the bioactive agent (e.g., oligonucleotide), to non-dividing cells. More specifically, the pharmaceutical composition (i.e., anionic liposomal formulation) can include DOPC/DOPG, wherein the ratio of DOPC to DOPG is about 88:12; an antisense oligonucleotide having a sequence of SEQ ID NO: 1 targeted to human p53 RNA or an antisense oligonucleotide having a sequence of SEQ ID NO:2 targeted to rat p53 mRNA; the monovalent cation Na⁺; and the buffer HEPES. Such a pharmaceutical composition can effectively encapsulate about 55% to about 65% of the antisense oligonucleotide and can effectively deliver the antisense oligonucleotide to non-dividing cells.

Anionic Liposome

In one embodiment of the present invention, the liposome employed is an anionic liposome. In such an embodiment, any suitable anionic lipid can be employed, provided the resulting anionic liposome has a net negative charge. Suitable anionic lipids are disclosed, e.g., Liposomes: from Physics to applications by D. D. Lasic, New York, Elsevier, 1993. Additionally, any suitable combination of lipids, (e.g., anionic, zwitterionic, and/or neutral) can be employed to provide an anionic liposome, provided the resulting anionic liposome has a net negative charge. Suitable zwitterionic and neutral lipids are disclosed, e.g., Liposomes: from Physics to applications by D. D. Lasic, New York, Elsevier, 1993; and Liposomes—A practical approach by R.R.C. New, Oxford University Press, New York, 1990.

As used herein, “liposome” refers to an aqueous compartment enclosed within phospholipid bilayers. The liposome is a closed vesicle, formed by a lipid bilayer enclosing an aqueous compartment. See, On-Line Medical Dictionary website (http://www.graylab.ac.uk). The interior of the liposome may be used to encapsulate exogenous materials or drugs for ultimate delivery into the cells by fusion with the cell or internalization of the entire liposome and its contents by the cell. The mechanism of cellular delivery of the encapsulated materials depends on the properties of the lipids used in the liposome formulation. See, Concise Dictionary of Biomedicine and Molecular Biology, Pei-Show Juo, CRC Press (Boca Raton, Fla.) 1995.

As used herein, “anionic liposome” refers to a liposome with a net negative charge;

“cation” refers to an ion with a net positive charge; and

“anion” refers to an ion with a net negative charge.

In another embodiment of the present invention, the anionic liposome includes a phospholipid with a head group that includes sn-glycero-phosphocholine; sn-glycero-phospho-rac-(1-glycerol); sn-glycero-phospho-L-serine; sn-glycero-3-phosphate; or a combination thereof; wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

In another embodiment of the present invention, the anionic liposome includes a phospholipid with a head group that includes sn-glycero-phosphocholine; sn-glycero-phospho-rac-(1-glycerol); sn-glycero-3-phosphate; or a combination thereof.

Specifically, the anionic liposome can be 1,2-dioleoyl-sn-glycero-3-phosphocholine (DOPC); 1,2-dioleoyl-sn-glycero-3-[phospho-rac-(1-gylcerol)] (DOPG); 1,2-dioleoyl-sn-glycero-3-[phospho-L-serine] (DOPS); 1,2-dioleoyl-sn-glycero-3-phosphate (DOPA); or a combination thereof.

Specifically, the anionic liposome can be DOPC/DOPG. More specifically, the anionic liposome can be DOPC/DOPG wherein the ratio of DOPC to DOPG is about 88:12.

As used herein, “DOPC” refers to 1,2-dioleoyl-sn-glycero-3-phosphocholine;

“DOPG” refers to 1,2-dioleoyl-sn-glycero-3-[phospho-rac-(1-glycerol)];

“DOPS” refers to 1,2-dioleoyl-sn-glycero-3-[phospho-L-serine];

“DOPA” refers to 1,2-dioleoyl-sn-glycero-3-phosphate;

“DOPE” refers to 1,2-dioleoyl-sn-glycero-3-phosphoethonolamine;

“DC-Chol” refers to 3β-[N-(N′,N′-dimethylaminoethane)-carbamol] cholesterol; and

“DOTAP” refers to 1,2-dioleoyl-3-trimethylammonium-propane.

As used herein, “phospholipid” refers to a lipid that consists of glycerol, fatty acids, phosphate, and an organic component (e.g., choline, ethanolamine, inositol, or sphingosine). See, Concise Dictionary of Biomedicine and Molecular Biology, Pei-Show Juo, CRC Press (Boca Raton, Fla.) 1995.

It is appreciated that those of skill in the art understand that a phospholipid with the head group sn-glycero-phosphocholine is a zwitterionic lipid. As such, when the liposome is anionic and includes a phospholipid with the head group sn-glycero-phosphocholine, the anionic liposome must also include at least one anionic lipid, in combination with the phospholipid having the head group sn-glycero-phosphocholine. Otherwise, the liposome would not be anionic. Suitable anionic lipids that can be employed in combination with the phospholipid having the head group sn-glycero-phosphocholine include, e.g., a phospholipid with a head group that includes sn-glycero-phospho-rac-(1-glycerol); sn-glycero-phospho-L-serine; sn-glycero-3-phosphate; or a combination thereof.

It is appreciated that those of skill in the art understand when an anionic liposome of the present invention includes two or more phospholipids, the two or more phospholipids are not conjugated to each other through a chemical bond.

The anionic liposome can include individual phospholipids in any suitable, effective, and appropriate amount. For example, the anionic liposome can include any combination of DOPC, DOPG, DOPS, and DOPA; wherein the amount of any one of DOPG, DOPS, and DOPA is up to about 100% of the anionic liposome and wherein the amount of DOPC is up to about 99%; provided the combined amount of each equals 100% of the anionic liposome, or less. Specifically, the amount of DOPC can be about 80% of the anionic liposome to about 95% of the anionic liposome and the amount of DOPG can be about 5% of the anionic liposome to about 20% of the anionic liposome.

In one embodiment of the present invention, a suitable amount of an additional lipid group can be employed to increase the stability of the pharmaceutical composition. Additional suitable lipid groups include, e.g., 2-9 wt. % cholesterol, 2-9 wt. % phosphotidyl ethanolamine (DOPE), 2-9 wt. % polyethylene glycol (PEG), or a combination thereof.

Additionally, the pharmaceutical composition can include the anionic liposome in any suitable, effective, and appropriate amount. For example, the anionic liposome can be present up to about 500 mM in the pharmaceutical composition or in about 2.5 mM to about 30 mM in the pharmaceutical composition. Typically, the amount of anionic liposome will depend in part upon the nature of the anionic liposome and/or the nature and amount of bioactive agent.

Specifically, when the bioactive agent has a molecular weight of about 250 to about 750, the anionic liposome can be present in about 5 mM to about 25 mM in the pharmaceutical composition.

Specifically, when the bioactive agent has a molecular weight of about 750 to about 1500, the anionic liposome can be present in about 7.5 mM to about 25 mM in the pharmaceutical composition.

Specifically, when the bioactive agent is an oligonucleotide having a length of about 5 bases to about 50 bases, the anionic liposome can be present in about 2.5 mM to about 25 mM in the pharmaceutical composition.

Specifically, when the bioactive agent is an oligonucleotide having a length of about 15 bases to about 30 bases, the anionic liposome can be present in about 2.5 mM to about 25 mM in the pharmaceutical composition.

Specifically, when the bioactive agent is a protein having a molecular weight of up to about 75,000; the anionic liposome can be present in about 2.5 mM to about 30 mM in the pharmaceutical composition.

Specifically, when the bioactive agent is a protein having a molecular weight of about 1,000 to about 5,000; the anionic liposome can be present in about 2.5 mM to about 30 mM in the pharmaceutical composition.

Specifically, when the bioactive agent is double or single stranded genetic material, or a fragment thereof having a molecular weight of up to about 1×10⁸; the anionic liposome can be present in about 2.5 mM to about 40 mM in the pharmaceutical composition.

Specifically, when the bioactive agent is double or single stranded genetic material, or a fragment thereof having a molecular weight of about 1×10⁵ to about 1×10⁷; the anionic liposome can be present in about 2.5 mM to about 40 mM in the pharmaceutical composition.

The anionic liposome can have any suitable, effective, and appropriate size (i.e., mean diameter). Specifically, the mean diameter of the anionic liposome can be about 20 nm to about 5 microns. More specifically, the mean diameter of the anionic liposome can be about 75 nm to about 500 nm. More specifically, the mean diameter of the anionic liposome can be about 175 nm to about 225 nm.

The pharmaceutical composition can include both the bioactive agent and the anionic liposome in any suitable, effective, and appropriate ratio. Typically, the molar ratio of bioactive agent to anionic liposome can be about 10:1 to about 1:1×1100. The ratio of bioactive agent to anionic liposome will typically depend in part upon the nature or each of the bioactive agent to anionic liposome. For example, the anionic liposome can be DOPC/DOPG wherein the ratio of DOPC to DOPG is about 88:12; and the bioactive agent can be an antisense oligonucleotide having a sequence of SEQ ID NO:1 targeted to human p53 mRNA or an antisense oligonucleotide having a sequence of SEQ ID NO:2 targeted to rat p53 mRNA. In such an embodiment, the molar ratio of bioactive agent to anionic liposome can be about 10:1 to about 1:1×10¹⁰. More specifically, in such an embodiment, the molar ratio of bioactive agent to anionic liposome can be about 5:1 to about 1:10,000.

Bioactive Agent

In one embodiment of the present invention, any suitable bioactive agent can be employed. Suitable bioactive agents are disclosed, e.g., in Physician's Desk Reference (PDR), 55 Edition, (2001); USP Dictionary of USAN and International Drug Names, 2000 Edition; Aldrich Handbook of Fine Chemicals, Aldrich (Milwaukee, Wis.) 2001; Sigma Catalogue of Biochemicals and Reagents, Sigma-Aldrich Co. (St. Louis, Mo.) 2001; U.S. Pat. No. 6,120,797; and Concise Dictionary of Biomedicine and Molecular Biology, Pei-Show Juo, CRC Press (Boca Raton, Fla.) 1995.

Suitable bioactive agents include, e.g. antiviral agents; antibacterial agents; antifungal agents; antineoplastic agents; anti-inflammatory agents; radiolabels; peptides; proteins; oligonucleotides; hormones; carbohydrates; growth factors; cytokines; radiopaque compounds; fluorescent compounds; mydriatic compounds; bronchodilator; local anesthetics; nucleic acid sequences; double or single stranded genetic material, or a fragment thereof; analgesics; antiparasitics; antipsychotics; antispasmodics; arthritis medications; biologicals; bone metabolism regulators; calcium channel blockers; cardiovascular agents; central nervous system stimulants; diabetes agents; diagnostics; fungal medications; gastrointestinal agents; histamine receptor antagonists; immunosuppressives; muscle relaxants; nausea medications; nucleoside analogues; parkinsonism drugs; platelet inhibitors; psychotropics; respiratory drugs; sedatives; urinary anti-infectives; urinary tract agents; vitamins; nucleotides; signaling molecules; fluorescent molecules; bioactive lipids; or a combination thereof.

Specifically, suitable bioactive agents include, e.g., antiviral agents such as acyclovir, zidovudine and the interferons; antibacterial agents such as aminoglycosides, cephalosporins and tetracyclines; antifungal agents such as polyene antibiotics, imidazoles and triazoles; antimetabolic agents such as folic acid, and purine and pyrimidine analogs; antineoplastic agents such as the anthracycline antibiotics and plant alkaloids; sterols such as cholesterol; carbohydrates, e.g., sugars and starches; amino acids, peptides, proteins such as cell receptor proteins, immunoglobulins, enzymes, hormones, neurotransmitters and glycoproteins; dyes; radiolabels such as radioisotopes and radioisotope-labeled compounds; radiopaque compounds; fluorescent compounds; mydriatic compounds; bronchodilator; local anesthetics; nucleic acid sequences such as messenger RNA, cDNA, genomic DNA and plasmids; and bioactive lipids such as ether lipids and ceramides.

Specifically, the bioactive agent can be double or single stranded genetic material, or a fragment thereof. More specifically, the bioactive agent can be an oligonucleotide (ON). In order to selectively inhibit just one mRNA species among a population of mRNAs present within a cell, the oligonucleotide should theoretically be at least 17 nucleotides long for humans (3×10⁹ base pairs in the genome; 60% of AT base pairs). These calculations assume a random distribution of nucleotides within mRNA species and that only 0.5% of the eukaryotic genome is transcribed. It is believed that ONs shorter than 15 bases often bind nonspecifically. Likewise, ONs longer than 30 bases might have decreased hybridization with the target mRNA. Thermodynamic analysis of oligonucleotide/target mRNA interactions showed that ONs of 15-19 bases have the highest selectivity towards the target mRNA (Monia et al., Selective inhibition of mutant Ha-ras mRNA expression by antisense oligonucleotides. J. Biol. Chem., 267, 19954-62 (1992)). 18 nucleotides can be employed since it fits 6 codons. Binding to an entire codon is thought to enhance the stability of the DNA/RNA hydrid. Moreover, ONs of 18 bases are often used in antisense studies.

More specifically, the bioactive agent can be a p53 antisense oligonucleotide. More specifically, the bioactive agent can be an antisense oligonucleotide having a sequence of SEQ ID NO:1 targeted to human p53 mRNA or an antisense oligonucleotide having a sequence of SEQ ID NO:2 targeted to rat p53 mRNA.

The bioactive agent can be present in any suitable, effective, and appropriate amount. For example, the bioactive agent can be present up to about 50 mM in the pharmaceutical composition, in about 1 femtoM to about 1 M in the pharmaceutical composition, or in about 2 nM to about 10 mM in the pharmaceutical composition. Typically, the amount of bioactive agent will depend in part upon the nature and amount of anionic liposome employed and/or the nature of the bioactive agent.

Specifically, the bioactive agent can have a molecular weight of about 250 to about 750. In such an embodiment, the bioactive agent can be present in about 0.5 mM to about 10 mM in the pharmaceutical composition.

Specifically, the bioactive agent can have a molecular weight of about 750 to about 1500. In such an embodiment, the bioactive agent can be present in about 0.5 mM to about 5 mM in the pharmaceutical composition.

Specifically, the bioactive agent can be an oligonucleotide having a length of about 5 bases to about 50 bases. In such an embodiment, the bioactive agent can be present in about 25 μM to about 250 μM in the pharmaceutical composition.

Specifically, the bioactive agent can be an oligonucleotide having a length of about 15 bases to about 30 bases. In such an embodiment, the bioactive agent can be present in about 25 μM to about 250 μM in the pharmaceutical composition.

Specifically, the bioactive agent can be a protein having a molecular weight of up to about 75,000. In such an embodiment, the bioactive agent can be present in about 25 μM to about 250 μM in the pharmaceutical composition.

Specifically, the bioactive agent can be a protein having a molecular weight of about 1,000 to about 5,000. In such an embodiment, the bioactive agent can be present in about 25 μM to about 250 μM in the pharmaceutical composition.

Specifically, the bioactive agent can be double or single stranded genetic material, or a fragment thereof, having a molecular weight of up to about 1×10⁸. In such an embodiment, the bioactive agent can be present in about 2 nM to about 40 nM in the pharmaceutical composition.

Specifically, the bioactive agent can be double or single stranded genetic material, or a fragment thereof having a molecular weight of about 1×10⁵ to about 1×10⁷. In such an embodiment, the bioactive agent can present in about 2 nM to about 40 nM in the pharmaceutical composition.

The anionic liposome effectively encapsulates at least a portion of the bioactive agent. Typically, up to about 100% of the bioactive agent can be encapsulated in the anionic liposome. Specifically, more than about 10% of the bioactive agent can be encapsulated in t he anionic liposome. More specifically, more than about 20% of the bioactive agent can be encapsulated in the anionic liposome. More specifically, more than about 50% of the bioactive agent can be encapsulated in the anionic liposome. The amount of encapsulation depends in part upon the nature and amount of anionic liposome and/or the nature and amount of the bioactive agent. For example, the anionic liposome can be DOPC/DOPG wherein the ratio of DOPC to DOPG is about 88:12; and the bioactive agent can be an antisense oligonucleotide having a sequence of SEQ ID NO: 1 targeted to human p53 mRNA or an antisense oligonucleotide having a sequence of SEQ ID NO:2 targeted to rat p53 mRNA. In such an embodiment, about 55% to about 60% of the bioactive agent can be encapsulated in the anionic liposome.

As used herein, “encapsulate” means to encase in or as if in a capsule; to enclose in or as if in a case.

The ratio of bioactive agent to anionic liposome will typically depend in part upon the nature of each of the bioctive agents. Typically, the molar ratio of bioactive agent to anionic liposome can be about 10:1 to about 1:1×10¹⁰. The ratio of bioactive agent to anionic liposome will typically depend in part upon the nature or each of the bioactive agent to anionic liposome. For example, the anionic liposome can be DOPC/DOPG wherein the ratio of DOPC to DOPG is about 88:12; and the bioactive agent can be an antisense oligonucleotide having a sequence of SEQ ID NO: 1 targeted to human p53 mRNA or an antisense oligonucleotide having a sequence of SEQ ID NO:2 targeted to rat p53 mRNA. In such an embodiment, the molar ratio of bioactive agent to anionic liposome can be about 10:1 to about 1:1×10¹⁰. More specifically, in such an embodiment, the molar ratio of bioactive agent to anionic liposome can be about 5:1 to about 1:10,000.

Cation

The pharmaceutical composition can include one or more (e.g., 1, 2, 3, or 4) suitable, appropriate, and effective cations. It is appreciated that those of skill in the art understand that when a cation is present in the pharmaceutical composition, the counter ion (i.e., the anion) will also be present in the pharmaceutical composition. For example, the suitable cation can be Na⁺. The presence of Na⁺ can be from, e.g., sodium chloride (NaCl). As such, the anion (e.g., Cl⁻) will also be present in the pharmaceutical composition.

The cation can be a monovalent cation or a divalent cation. Specifically, the cation can be a divalent cation, e.g., Mg²⁺ or Ca²⁺. Specifically, the cation can be a monovalent cation. Suitable monovalent cations include, e.g., Na⁺, K⁺, Li⁺, Fr⁺, Rb⁺, Cs⁺, choline, and N-methylglucamine. More specifically, the cation can be Na⁺, K⁺, or Li⁺. More specifically, the cation can be Na⁺ or K⁺. More specifically, the cation can be Na⁺.

The cation can be present in any suitable, appropriate, and effective amount. Specifically, the cation can be present below about 100 mM in the pharmaceutical composition, below about 50 mM in the pharmaceutical composition, or below about 25 mM in the pharmaceutical composition. More specifically, the cation can be present up to about 50 mM in the pharmaceutical composition, up to about 15 mM in the pharmaceutical composition, or up to about 5 mM in the pharmaceutical composition.

Buffer

The pharmaceutical composition can include one or more (e.g., 1, 2, 3, or 4) suitable, effective, and appropriate buffers. The buffer can maintain the pH of the pharmaceutical composition in a suitable range. Typically, the buffer can maintain the pH of the pharmaceutical composition between about 5.5 to about 8.5. Specifically, the buffer can maintain the pH of the pharmaceutical composition between about 6.0 to about 8.0. More specifically, the buffer can maintain the pH of the pharmaceutical composition between about 7.0 to about 7.5. As such, the buffer can maintain the pH of the pharmaceutical composition at or near a physiological pH.

The buffer can be present in any suitable, effective, and appropriate amount. Specifically, the buffer can be present up to about 50 mM in the pharmaceutical composition. More specifically, the buffer can be present up to about 10 mM in the pharmaceutical composition.

Any suitable, effective, and appropriate buffer can be employed. Suitable, effective, and appropriate buffers include, e.g., HEPES; BES; HEPPS; imidazole; MOPS; TES; TEA; monobasic or dibasic potassium phosphate; monobasic or dibasic sodium phosphate; cacodylic acid; MES; PIPES; glycine amide; glycylglycine; TAPS; boric acid; BIS-TRIS PROPANE; DIPSO; TAPSO; HEPPSO; POPSO; EPPS; TRICINE; BICINE; TAPS; pharmaceutically acceptable salt thereof; and combinations thereof. Specifically, the buffer can be [4-(2-hydroxyethyl)-1-piperazine ethanesulfonic acid] (HEPES). Other suitable, effective, and appropriate buffers can be found, e.g., at Sigma Catalogue of Biochemicals and Reagents, Sigma-Aldrich Co. (St. Louis, Mo.) 2001.

Target

The pharmaceutical composition of the present invention can be targeted to any suitable target or site in animal tissue. Specifically, the animal can be a mammal (e.g., human). The target can be pleuripotent tissue, e.g., stem cells, embryonic stem cells, or bone marrow-derived stem cells. The target can also be non-pleuripotent tissue. Suitable targets include, e.g., cells of organs such as the brain, central and peripheral nervous systems, liver, lung, larynx, bone marrow, spleen, kidney, lymphatic system, hematopoetic system, gastric mucosa, small and large intestines, gall bladder, pancreas, salivary glands, testes, ovary, cervix, uterus, muscle, skin, thyroid gland, parathyroid gland, adrenal gland, connective tissue, chondroid tissue, blood vessels, macrophages, pleura, and placenta. Specifically, the target can be non-dividing cells (e.g., neuronal cells). More specifically, the neuronal cells can be hippocampal neuronal cells. Additionally, the suitable target can include a tumor or a growth.

In one embodiment of the present invention, the pharmaceutical composition of the present invention can be delivered to cells that express a receptor belonging to the low-density lipoprotein (LDL) gene family. More specifically, the pharmaceutical composition of the present invention can be delivered to cells that possess a low-density lipoprotein receptor-related protein (LRP) receptor. More specifically, the pharmaceutical composition of the present invention can be delivered to cells that possess an endocytic low-density lipoprotein receptor-related protein receptor. More specifically, the pharmaceutical composition of the present invention can be delivered to cells that possess a receptor that is expressed in mammalian central nervous system (CNS). More specifically, the pharmaceutical composition of the present invention can be delivered to neuronal cells. More specifically, the pharmaceutical composition of the present invention can be delivered to hippocampal neuronal cells.

Embodiments

The following are exemplary embodiments of the present invention:

[1] One embodiment of the present invention provides for a pharmaceutical composition comprising: (a) an anionic liposome comprising a phospholipid with a head group selected from the group consisting of sn-glycero-phosphocholine; sn-glycero-phospho-rac-(1-glycerol); sn-glycero-phospho-L-serine; sn-glycero-3-phosphate; and combinations thereof; (b) a bioactive agent; and (c) a cation, a buffer, or a combination thereof; wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

[2] Another embodiment of the present invention provides a pharmaceutical composition comprising: (a) an anionic liposome comprising a phospholipid with a head group selected from the group consisting of sn-glycero-phosphocholine; sn-glycero-phospho-rac-(1-glycerol); sn-glycero-3-phosphate; and combinations thereof; (b) a bioactive agent; and (c) a cation, a buffer, or a combination thereof.

[3] Another embodiment of the present invention provides a pharmaceutical composition comprising: (a) an anionic liposome; (b) a bioactive agent is an antisense oligonucleotide having a sequence of SEQ ID NO:1 targeted to human p53 mRNA or an antisense oligonucleotide having a sequence of SEQ ID NO:2 targeted to rat p53 mRNA; and (c) a cation, a buffer, or a combination thereof.

[4] Another embodiment of the present invention provides a pharmaceutical composition of any one of embodiments [1]-[3] wherein the anionic liposome is 1,2-dioleoyl-sn-glycero-3-phosphocholine (DOPC); 1,2-dioleoyl-sn-glycero-3-[phospho-rac-(1-gylcerol)] (DOPG); 1,2-dioleoyl-sn-glycero-3-[phospho-L-serine] (DOPS); 1,2-dioleoyl-sn-glycero-3-phosphate (DOPA); or a combination thereof.

[5] Another embodiment of the present invention provides a pharmaceutical composition of any one of embodiments [1]-[4] wherein the anionic liposome is DOPC/DOPG.

[6] Another embodiment of the present invention provides a pharmaceutical composition of any one of embodiments [1]-[5] wherein the anionic liposome is DOPC/DOPG wherein the ratio of DOPC to DOPG is about 88:12.

[7] Another embodiment of the present invention provides a pharmaceutical composition of any one of embodiments [1]-[6] wherein the mean diameter of the anionic liposome is about 20 nm to about 5 microns.

[8] Another embodiment of the present invention provides a pharmaceutical composition of any one of embodiments [1]-[7] wherein the mean diameter of the anionic liposome is about 75 nm to about 500 nm.

[9] Another embodiment of the present invention provides a pharmaceutical composition of any one of embodiments [1]-[8] wherein the mean diameter of the anionic liposome is about 175 nm to about 225 nm.

[10] Another embodiment of the present invention provides a pharmaceutical composition of any one of embodiments [1]-[9] wherein the anionic liposome is present up to about 500 mM in the pharmaceutical composition.

[11] Another embodiment of the present invention provides a pharmaceutical composition of any one of embodiments [1]-[10] wherein the anionic liposome is present in about 2.5 mM to about 30 mM in the pharmaceutical composition.

[12] Another embodiment of the present invention provides a pharmaceutical composition of any one of embodiments [1]-[11] wherein the bioactive agent has a molecular weight of about 250 to about 750 and the anionic liposome is present in about 5 mM to about 25 mM in the pharmaceutical composition.

[13] Another embodiment of the present invention provides a pharmaceutical composition of any one of embodiments [1]-[11] wherein the bioactive agent has a molecular weight of about 750 to about 1500 and the anionic liposome is present in about 7.5 mM to about 25 mM in the pharmaceutical composition.

[14] Another embodiment of the present invention provides a pharmaceutical composition of any one of embodiments [1]-[11] wherein the bioactive agent is an oligonucleotide having a length of about 5 bases to about 50 bases and the anionic liposome is present in about 2.5 mM to about 25 mM in the pharmaceutical composition.

[15] Another embodiment of the present invention provides a pharmaceutical composition of any one of embodiments [1]-[11] wherein the bioactive agent is an oligonucleotide having a length of about 15 bases to about 30 bases and the anionic liposome is present in about 2.5 mM to about 25 mM in the pharmaceutical composition.

[16] Another embodiment of the present invention provides a pharmaceutical composition of any one of embodiments [1]-[11] wherein the bioactive agent is a protein having a molecular weight of up to about 75,000 and the anionic liposome is present in about 2.5 mM to about 30 mM in the pharmaceutical composition.

[17] Another embodiment of the present invention provides a pharmaceutical composition of any one of embodiments [1]-[11] wherein the bioactive agent is a protein having a molecular weight of about 1,000 to about 5,000 and the anionic liposome is present in about 2.5 mM to about 30 mM in the pharmaceutical composition.

[18] Another embodiment of the present invention provides a pharmaceutical composition of any one of embodiments [1]-[11] wherein the bioactive agent is double or single stranded genetic material, or a fragment thereof having a molecular weight of up to about 1×10⁸ and the anionic liposome is present in about 2.5 mM to about 40 mM in the pharmaceutical composition.

[19] Another embodiment of the present invention provides a pharmaceutical composition of any one of embodiments [1]-[11] wherein the bioactive agent is double or single stranded genetic material, or a fragment thereof having a molecular weight of about 1×10⁵ to about 1×10⁷ and the anionic liposome is present in about 2.5 mM to about 40 mM in the pharmaceutical composition.

[20] Another embodiment of the present invention provides a pharmaceutical composition of any one of embodiments [1]-[19] wherein the bioactive agent is an antiviral agent; an antibacterial agent; an antifungal agent; an antineoplastic agent; an anti-inflammatory agent; a radiolabel; a peptide; a protein; an oligonucleotide; a hormone; a carbohydrate; a growth factor; a cytokine; a radiopaque compound; a fluorescent compound; a mydriatic compound; a bronchodilator; a local anesthetic; a nucleic acid sequence; double or single stranded genetic material, or a fragment thereof; RNAi; a ribozyme; a transposon; a chimeraplast; an analgesic; an antiparasitic; an antipsychotic; an antispasmodic; an arthritis medication; a biological; a bone metabolism regulator; a calcium channel blocker; a cardiovascular agent; a central nervous system stimulant; a diabetes agent; a diagnostic; a fungal medication; a gastrointestinal agent; a histamine receptor antagonist; an immunosuppressive; a muscle relaxant; a nausea medication; a nucleoside analogue; a parkinsonism drug; a platelet inhibitor; a psychotropic; a respiratory drug; a sedative; a urinary anti-infective; a urinary tract agent; a vitamin; a nucleotide; a signaling molecule; a fluorescent molecule; a bioactive lipid; a neuroactive agent; an energy substrate; or a combination thereof.

[21] Another embodiment of the present invention provides a pharmaceutical composition of any one of embodiments [1]-[20] wherein the bioactive agent is RNAi; a ribozyme; a transposon; a chimeraplast; double or single stranded genetic material; or a fragment thereof.

[22] Another embodiment of the present invention provides a pharmaceutical composition of any one of embodiments [1]-[21] wherein the bioactive agent is a p53 antisense oligonucleotide.

[23] Another embodiment of the present invention provides a pharmaceutical composition of any one of embodiments [1]-[22] wherein the bioactive agent is an antisense oligonucleotide having a sequence of SEQ ID NO:1 targeted to human p53 mRNA or an antisense oligonucleotide having a sequence of SEQ ID NO:2 targeted to rat p53 mRNA.

[24] Another embodiment of the present invention provides a pharmaceutical composition of any one of embodiments [1]-[23] wherein the bioactive agent is present up to about 50 mM in the pharmaceutical composition.

[25] Another embodiment of the present invention provides a pharmaceutical composition of any one of embodiments [1]-[24] wherein the bioactive agent is present in about 1 femtoM to about 1 M in the pharmaceutical composition.

[26] Another embodiment of the present invention provides a pharmaceutical composition of any one of embodiments [1]-[25] wherein the bioactive agent is present in about 2 nM to about 10 mM in the pharmaceutical composition.

[27] Another embodiment of the present invention provides a pharmaceutical composition of any one of embodiments [1]-[11] wherein the bioactive agent has a molecular weight of about 250 to about 750 and is present in about 0.5 mM to about 10 mM in the pharmaceutical composition.

[28] Another embodiment of the present invention provides a pharmaceutical composition of any one of embodiments [1]-[11] wherein the bioactive agent has a molecular weight of about 750 to about 1500 and is present in about 0.5 mM to about 5 nM in the pharmaceutical composition.

[29] Another embodiment of the present invention provides a pharmaceutical composition of any one of embodiments [1]-[11] wherein the bioactive agent is an oligonucleotide having a length of about 5 bases to about 50 bases and is present in about 25 μM to about 250 μM in the pharmaceutical composition.

[30] Another embodiment of the present invention provides a pharmaceutical composition of any one of embodiments [1]-[11] wherein the bioactive agent is an oligonucleotide having a length of about 15 bases to about 30 bases and is present in about 25 μM to about 250 μM in the pharmaceutical composition.

[31] Another embodiment of the present invention provides a pharmaceutical composition of any one of embodiments [1]-[11] wherein the bioactive agent is a protein having a molecular weight of up to about 75,000 and is present in about 25 μM to about 250 μM in the pharmaceutical composition.

[32] Another embodiment of the present invention provides a pharmaceutical composition of any one of embodiments [1]-[11] wherein the bioactive agent is a protein having a molecular weight of about 1,000 to about 5,000 and is present in about 25 μM to about 250 μM in the pharmaceutical composition.

[33] Another embodiment of the present invention provides a pharmaceutical composition of any one of embodiments [1]-[11] wherein the bioactive agent is double or single stranded genetic material, or a fragment thereof, having a molecular weight of up to about 1×10⁸ and is present in about 2 nM to about 40 nM in the pharmaceutical composition.

[34] Another embodiment of the present invention provides a pharmaceutical composition of any one of embodiments [1]-[11] wherein the bioactive agent is double or single stranded genetic material, or a fragment thereof having a molecular weight of about 1×10⁵ to about 1×10⁷ and is present in about 2 nM to about 40 nM in the pharmaceutical composition.

[35] Another embodiment of the present invention provides a pharmaceutical composition of any one of embodiments [1]-[34] wherein up to about 100% of the bioactive agent is encapsulated in the anionic liposome.

[36] Another embodiment of the present invention provides a pharmaceutical composition of any one of embodiments [I]-[34] wherein more than about 10% of the bioactive agent is encapsulated in the anionic liposome.

[37] Another embodiment of the present invention provides a pharmaceutical composition of any one of embodiments [1]-[34] wherein more than about 20% of the bioactive agent is encapsulated in the anionic liposome.

[38] Another embodiment of the present invention provides a pharmaceutical composition of any one of embodiments [1]-[34] wherein about 55% to about 60% of the bioactive agent is encapsulated in the anionic liposome.

[39] Another embodiment of the present invention provides a pharmaceutical composition of any one of embodiments [1]-[38] wherein the cation is a monovalent cation.

[40] Another embodiment of the present invention provides a pharmaceutical composition of any one of embodiments [1]-[38] wherein the cation is Na⁺, K⁺, Li⁺, Fr⁺, Rb⁺, or Cs⁺.

[41] Another embodiment of the present invention provides a pharmaceutical composition of any one of embodiments [1]-[38] wherein the cation is Na⁺, K⁺, or Li⁺.

[42] Another embodiment of the present invention provides a pharmaceutical composition of any one of embodiments [1]-[41] wherein the cation is present up to about 50 MM in the pharmaceutical composition.

[43] Another embodiment of the present invention provides a pharmaceutical composition of any one of embodiments [1]-[42] wherein the cation is present up to about 5 mM in the pharmaceutical composition.

[44] Another embodiment of the present invention provides a pharmaceutical composition of any one of embodiments [1]-[43] wherein the buffer maintains the pH of the pharmaceutical composition between about 6.0 to about 8.0.

[45] Another embodiment of the present invention provides a pharmaceutical composition of any one of embodiments [1]-[43] wherein the buffer maintains the pH of the pharmaceutical composition between about 7.0 to about 7.5.

[46] Another embodiment of the present invention provides a pharmaceutical composition of any one of embodiments [1]-[45] wherein the buffer is HEPES; BES; HEPPS; imidazole; MOPS; TES; TEA; monobasic or dibasic potassium phosphate; monobasic or dibasic sodium phosphate; cacodylic acid; MES; PIPES; glycine amide; glycylglycine; TAPS; boric acid; BIS-TRIS PROPANE; DIPSO; TAPSO; HEPPSO; POPSO; EPPS; TRICINE; BICINE; TAPS; a pharmaceutically acceptable salt thereof; or a combination thereof.

[47] Another embodiment of the present invention provides a pharmaceutical composition of any one of embodiments [1]-[45] wherein the buffer is [4-(2-hydroxyethyl)-1-piperazine ethanesulfonic acid] (HEPES).

[48] Another embodiment of the present invention provides a pharmaceutical composition of any one of embodiments [1]-[47] wherein the buffer is present up to about 50 mM in the pharmaceutical composition.

[49] Another embodiment of the present invention provides a pharmaceutical composition of any one of embodiments [1]-[47] wherein the buffer is present up to about 10 mM in the pharmaceutical composition.

[50] Another embodiment of the present invention provides a pharmaceutical composition of any one of embodiments [1]-[49] wherein the molar ratio of bioactive agent to anionic liposome is about 10:1 to about 1:1×10¹⁰.

[51] Another embodiment of the present invention provides a pharmaceutical composition of any one of embodiments [1]-[49] wherein the molar ratio of bioactive agent to anionic liposome is about 5:1 to about 1:10,000.

[52] Another embodiment of the present invention provides a method of delivering a bioactive agent to a target comprising contacting the target with a pharmaceutical composition of any one of embodiments [1]-[51].

[53] Another embodiment of the present invention provides a method of delivering a bioactive agent to a target comprising contacting the target with a composition, wherein the composition comprises: (a) an anionic liposome comprising a phospholipid with a head group selected from the group consisting of sn-glycero-phosphocholine; sn-glycero-phospho-rac-(1-glycerol); sn-glycero-phospho-L-serine; sn-glycero-3-phosphate; and combinations thereof; (b) a bioactive agent; and (c) a cation, a buffer, or a combination thereof; wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

[54] Another embodiment of the present invention provides a method of delivering a bioactive agent to a target comprising contacting the target with a composition, wherein the composition comprises: (a) an anionic liposome comprising a phospholipid with a head group selected from the group consisting of sn-glycero-phosphocholine; sn-glycero-phospho-rac-(1-glycerol); sn-glycero-3-phosphate; and combinations thereof; (b) a bioactive agent; and (c) a cation, a buffer, or a combination thereof.

[55] Another embodiment of the present invention provides a method of delivering a bioactive agent to a target comprising contacting the target with a composition, wherein the composition comprises: (a) an anionic liposome; (b) a bioactive agent is an antisense oligonucleotide having a sequence of SEQ ID NO:1 targeted to human p53 mRNA or an antisense oligonucleotide having a sequence of SEQ ID NO:2 targeted to rat p53 mRNA; and (c) a cation, a buffer, or a combination thereof.

[56] Another embodiment of the present invention provides a method of delivering a bioactive agent to non dividing cells comprising contacting the non dividing cells with a composition, wherein the composition comprises: (a) an anionic liposome; (b) a bioactive agent; and (c) a cation, a buffer, or a combination thereof.

[57] Another embodiment of the present invention provides a method of any one of embodiments [52]-[56] wherein the target is a cell of an organ.

[58] Another embodiment of the present invention provides a method of any one of embodiments [52]-[56] wherein the target is stem cells, embryonic stem cells, bone marrow derived stem cells, pleuripotent tissue, or a cell of: the brain, central nervous system, peripheral nervous systems, liver, lung, larynx, bone marrow, spleen, kidney, lymphatic system, hematopoetic system, gastric mucosa, small intestine, large intestine, gall bladder, pancreas, salivary gland, teste, ovary, cervix, uterus, muscle, skin, thyroid gland, parathyroid gland, adrenal gland, connective tissue, chondroid tissue, blood vessel, macrophage, pleura, placenta, a tumor, or a growth.

[59] Another embodiment of the present invention provides a method of any one of embodiments [52]-[56] wherein the target is non-dividing cells.

[60] Another embodiment of the present invention provides a method of any one of embodiments [52]-[56] wherein the target is neuronal cells.

[61] Another embodiment of the present invention provides a method of any one of embodiments [52]-[56] wherein the target is hippocampal neuronal cells.

[62] Another embodiment of the present invention provides a method of any one of embodiments [52]-[56] wherein the target is a cell that expresses a receptor belonging to the low-density lipoprotein (LDL) gene family.

[63] Another embodiment of the present invention provides a method of any one of embodiments [52]-[56] wherein the target is a cell that express the low-density lipoprotein receptor-related protein (LRP) receptor.

[64] Another embodiment of the present invention provides a method of any one of embodiments [52]-[56] wherein the target is a cell that possesses an endocytic low-density lipoprotein receptor-related protein receptor.

[65] Another embodiment of the present invention provides a method of any one of embodiments [52]-[56] wherein the target is a cell that possesses a receptor that is expressed in mammalian central nervous system (CNS).

Preparation of the Pharmaceutical Composition

The pharmaceutical composition of the present invention (i.e., the liposomal formulation) can be prepared in any suitable, effective, and appropriate manner. For example, the pharmaceutical composition of the present invention can be prepared in any manner known to those of skill in the art. Specifically, the pharmaceutical composition of the present invention can be prepared as described in any one or more of U.S. Pat. Nos. 6,120,797; 4,880,635; 5,077,056; 5,399,331; 4,885,172; 5,059,421; 5,171,578; 4,522,803; 4,588,578; 5,030,453; 5,169,637; 4,975,282; EP Patent No. 510,086; U.S. Pat. Nos. 4,235,871; 5,008,050; and 5,059,421. Specifically, the pharmaceutical composition of the present invention can be prepared as described in the enclosed Ph.D. thesis titled Delivery of Antisense Oligonucleotides to Neurons by Anionic Liposomes: Therapeutic Potential and Mechanisms of Endocytosis (April, 2001).; Lasic, D. D. 1993. Preparation of liposomes. In Liposomes : from physics to applications. Elsevier, Amsterdam. 106-107; MacDonald, R. C., R. I. MacDonald, B. P. Menco, K. Takeshita, N. K. Subbarao, and L. R. Hu. 1991. Small-volume extrusion apparatus for preparation of large, unilamellar vesicles.

Biochim Biophys Acta. 1061:297-303; Olson, F., C. A. Hunt, F. C. Szoka, W. J. Vail, and D. Papahadjopoulos. 1979. Preparation of liposomes of defined size distribution by extrusion through polycarbonate membranes. Biochim Biophys Acta. 557:9-23; Szoka, F., Jr., and D. Papahadjopoulos. 1978. Procedure for preparation of liposomes with large internal aqueous space and high capture by reverse-phase evaporation. Proc Natl Acad Sci USA. 75:4194-8; Szoka, F., Jr., and D. Papahadjopoulos. 1980. Comparative properties and methods of preparation of lipid vesicles (liposomes). Annu Rev Biophys Bioeng. 9:467-508; and Wilschut, J. 1982. Preparation and properties of phospholipid vesicles. In Liposome methodology in pharmacology and biology. Vol. 107. L. D. Leserman and J. Barbet, editors. INSERM, Paris. 10-24.

Delivery of the Pharmaceutical Composition

The pharmaceutical composition of the present invention (i.e., the liposomal formulation) can be delivered in any suitable, effective, and appropriate manner. For example, the pharmaceutical composition of the present invention can be delivered in any manner known to those of skill in the art. Specifically, the pharmaceutical composition of the present invention can be delivered as described in any one or more of U.S. Pat. Nos. 6,120,797; 4,880,635; 5,077,056; 5,399,331; 4,885172; 5,059,421; 5,171,578; 4,522,803; 4,588,578; 5,030,453; 5,169,637; 4,975,282; EP Patent No. 510,086; U.S. Pat. Nos. 4,235,871; 5,008,050; and 5,059,421. Specifically, the pharmaceutical composition of the present invention can be delivered as described in the enclosed Ph.D. thesis titled Delivery of Antisense Oligonucleotides to Neurons by Anionic Liposomes: Therapeutic Potential and Mechanisms of Endocytosis (April, 2001).

The pharmaceutical composition of the present invention can be administered to a mammalian host, such as a human patient in a variety of forms adapted to the chosen route of administration, i.e., orally or parenterally, by intravenous, intramuscular, topical, subcutaneous, intracerebral, intracerebroventricular, intrathecal, and intraarterial routes.

Thus, the present pharmaceutical compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the pharmaceutical composition may be combined with one or more excipients and used in the form of ingestible tablets, buccal tablets, troches, capsules, elixirs, suspensions, syrups, wafers, and the like. Such compositions and preparations should contain at least 0.1% of the pharmaceutical composition. The percentage of the compositions and preparations may, of course, be varied and may conveniently be between about 2 to about 60% of the weight of a given unit dosage form. The amount of pharmaceutical composition in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the pharmaceutical composition may be incorporated into sustained-release preparations and devices.

The present compounds may also be administered intravenously or intraperitoneally by infusion or injection. Solutions of a compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising a pharmaceutical composition of the present invention adapted for the extemporaneous preparation of sterile injectable or infusible solutions or dispersions. The ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the labeled or unlabeled compound of the present invention plus any additional desired ingredient present in the previously sterile-filtered solutions.

For topical administration, the present pharmaceutical compositions may be applied to the skin in combination with a dermatologically acceptable carrier, which may be a solid or a liquid.

Useful dosages of the pharmaceutical compositions of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the pharmaceutical compositions of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%. Single dosages for injection, infusion or ingestion will generally vary between 50-1500 mg, and may be administered, i.e., 1-3 times daily, to yield levels of about 0.5-50 mg/kg, for adults.

The enclosed Ph.D. thesis titled Delivery of Antisense Oligonucleotides to Neurons by Anionic Liposomes: Therapeutic Potential and Mechanisms of Endocytosis (April, 2001), forms part of the present application.

All publications, references, catalogues, books, websites, patents, and patent documents cited herein are incorporated by reference herein, as though individually incorporated by reference.

The invention will now be illustrated by the following non-limiting examples:

### EXAMPLE 1

Anionic Liposomes Facilitate Widespread ON Delivery and Transgene Expression in Neurons and Other Cells Types.

Experimental Procedures

Oligonucleotide Design and Synthesis. An 18-mer oligonucleotide (5′-CTGTGAATCCTCCATGAC-3′, SEQ ID NO:2) that targets the translation initiation site of the rat p53 mRNA and is complementary to nucleotides 21 to 38 (GenBank accession number X13058 (Soussi et al., 1988)) was designed for this study. Oligonucleotides were synthesized and labeled at the 5′-end with Cy3 by Integrated DNA Technologies, Coralville, Iowa. The Cy3-labeled oligonucleotides were purified by reverse-phase HPLC to remove free dye. The oligonucleotides were reconstituted in sterile, nuclease-free Tris-EDTA buffer (pH 7.2) and stored at −20° C.

Liposome Preparation and Characterization. Dioleoyl phosphatidylcholine (DOPC), dioleoyl phosphatidylglycerol (DOPG), dioleoyl phosphatidylethanolamine (DOPE), dimethylaminoethane carbamoyl cholesterol (DC-Chol) and the headgroup labeled lipid Lissamine Rhodamine DOPE (N-Rh-DOPE) were purchased from Avanti Polar Lipids, Alabaster, Ala. and stored at −20° C. as stock solutions of 2 mg/ml in chloroform. Anionic liposomes encapsulating Cy3-oligonucleotides (AL-Cy3ONs) were prepared by a modification of the classic film hydration-extrusion procedure. Briefly, a lipid mixture of DOPC and DOPG was dried to a thin film under a stream of high-purity nitrogen, hydrated with a solution of Cy3ONs in 10 mM HEPES, 5 mM NaCl buffer (pH 7.4). After complete hydration, the suspension was transferred to a LIPOSOFAST miniextruder system (Avestin, Inc., Ottawa, Canada) and extruded through a series of polycarbonate membranes down to a pore size of 0.2 μm. Unencapsulated Cy3ONs were removed by minicolumn centrifugation using a Sephadex G-50 column. Liposomes were eluted in the void volume and unencapsulated Cy3ONs were eluted in subsequent fractions. Purified liposomes were stored at 4° C. until use. Cationic DC-Chol/DOPE (1/1 molar ratio) liposomes were prepared in 10 mM HEPES, 5 mM NaCl buffer and extruded to 200 nm. The liposomes were diluted in 5% w/v glucose, complexed with Cy3ONs in various charge ratios and used immediately after complex formation.

Hippocampal Cell Culture. Primary cultures of hippocampal neurons were prepared from neonatal rat pups (P1 or P2) as previously described (Dubinsky, 1993). Neurons were plated onto 22-mm square glass coverslips coated with collagen and polylysine, at a density of 150,000 cells per coverslip. Neurons were cultured for 24-30 hours in minimum essential medium (MEM) with 10% NUSERUM (Collaborative Research) to increase attachment to the glass substrate, after which the growing medium was replaced with Neurobasal medium with B27 supplements (Life Technologies, Rockville, Md.). Fluorodeoxyuridine (15 μg/ml) was added to restrict the growth of non-neuronal cells. Cultures were maintained at 37° C. in a humidified atmosphere containing 5% CO₂ for 6-9 days before use.

Uptake of Cy3-ONs by hippocampal neurons. Neurons were incubated with 1 μM Cy3ONs either free, encapsulated in anionic liposomes or as complexes with cationic liposomes, for various time periods at 37° C. Uninternalized ONs, liposomes or complexes were removed by several washes with L15 (Leibovitz's) medium. Cells were then fixed in 4% paraformaldehyde for 15 minutes at room temperature. For low temperature studies, neurons were incubated at 4° C. with AL-Cy3ONs for 10 minutes and then fixed. The cells were imaged immediately after fixation to avoid a potential redistribution of ONs (Grzanna et al., 1998).

Results

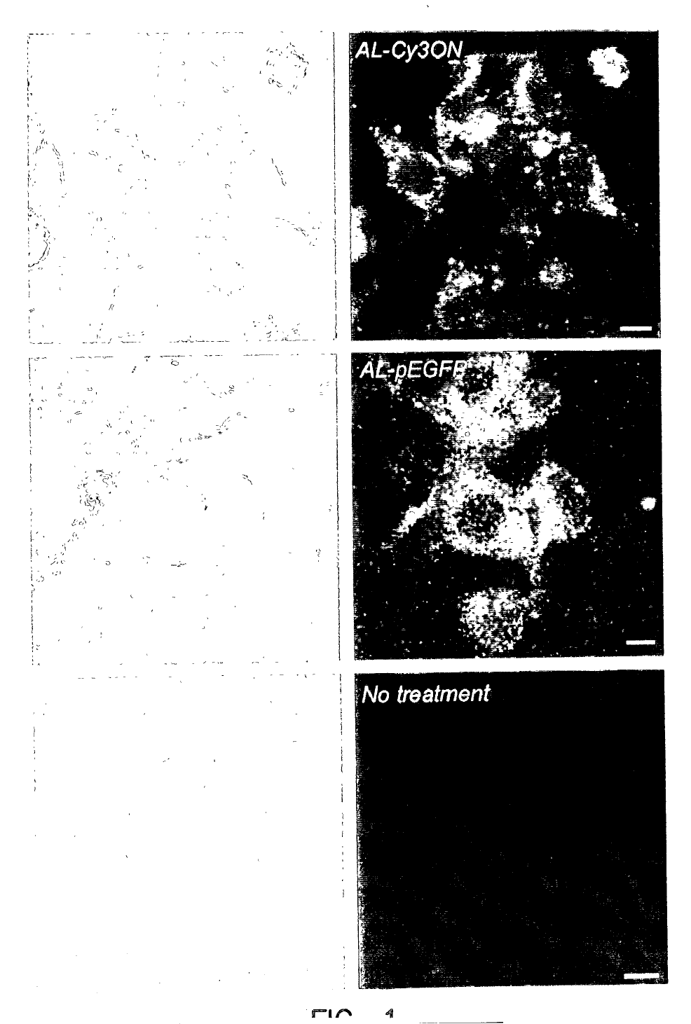

Following a 1 h incubation of neurons with 1 μM Cy3-labeled oligonucleotides encapsulated in anionic DOPC/DOPG liposomes (AL-Cy3ON) in serum-containing medium, Cy3 fluorescence was visible in punctate structures in the cytoplasm and in a diffuse manner in the nuclei of all neurons (FIG. 1, AL-Cy3ON). Uptake of AL-Cy3ONs was similar in serum-free medium (data not shown). Cy3-oligonucleotides without a delivery vector or complexed with preformed cationic DC-Chol/DOPE liposomes were taken up by a low percentage of neurons (Table 1). The net charge on the cationic lipid/Cy3ON complex did not influence uptake in the time period studied. Anionic liposomes were also capable of delivering plasmids to neurons and eliciting protein expression. Transfection with pEGFP-N1 encapsulated in anionic liposomes resulted in EGFP expression in neurons (FIG. 1, AL-pEGFP). To encapsulate pEGFP-N1 in the liposomes, the plasmid was collapsed with PEI prior to encapsulation. pEGFP-N1 alone or complexed with PEI was not successful in transfecting neurons and equivalent to untreated controls. These data show anionic liposomes facilitate widespread oligonucleotide delivery and transgenic expression in neurons.

### EXAMPLE 2

Delivery of Cy3-ONs by Anionic Liposomes to a Variety of Cell Types is Rapid and Uniform.

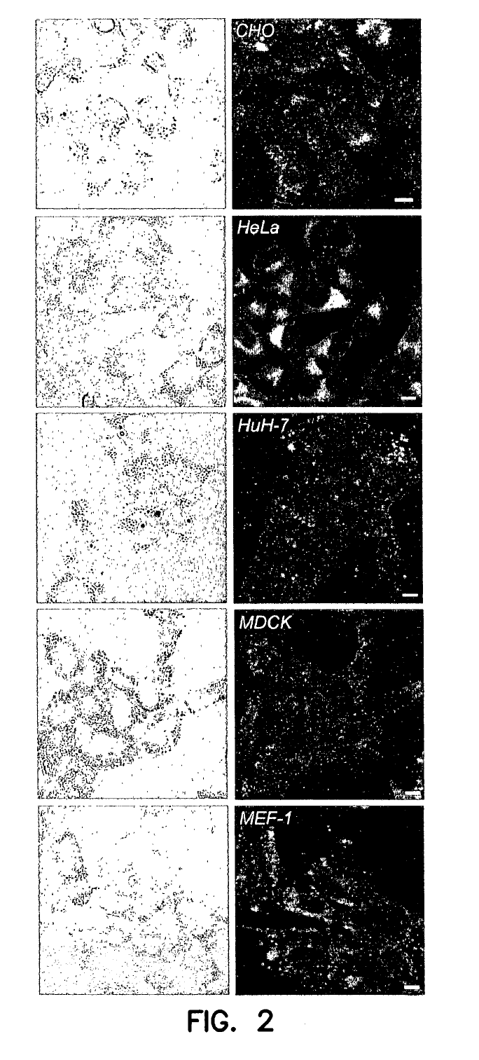

The ability of this delivery system to deliver DNA to cell types other than neurons was investigated by studying uptake of Cy3-oligonucleotides delivered by anionic DOPC/DOPG liposomes in cells derived from a variety of tissues. CHO, HeLa, HuH-7, MDCK and MEF-1 cells all avidly internalized AL-Cy3ONs within 1 h of incubation (FIG. 2). Uptake of AL-Cy3ONs occurred in the presence of serum in the culture medium. Similar to the uniform uptake seen in primary neurons, Cy3 fluorescence was visible in almost all cells exposed to AL-Cy3ONs in all cell types studied (Table 1). On the other hand, uptake of Cy3ON either without a delivery vector or complexed to cationic DC-Chol/DOPE liposomes was cell type-dependent, ranging from ˜5% in MDCK cells to ˜50% in HeLa cells (Table 1). These data show that delivery of Cy3-ONs by anionic liposomes to a variety of cell types is rapid and uniform.

TABLE 1
Comparison of the uptake of Cy3-ONs delivered
by anionic liposomes and cationic lipids

| | Percent of cells with | | |
|---|---|---|---|
| | intracellular Cy3 fluorescence | | |
| | Cy3-ONs | Cy3-ONs | |
| | encapsulated | complexed | Cy3-ONs |
| | in anionic | with cationic | without |
| | DOPC/DOPG | DC-Chol/DOPE | delivery |
| Cell Type | liposomes^{§} | liposomes* | system* |
| Primary rat | 100 | 9 | 6 |
| hippocampal neurons | | | |
| Chinese hamster | 99 ± 1.7 | 41.5 | 17 |
| ovary cell line | | | |
| (CHO-K1) | | | |
| Human cervical | 100 | 50.3 | 51.5 |
| carcinoma (HeLa) | | | |
| Human hepatoma | 97.8 ± 2 | 31.5 | 18.3 |
| (Huh-7) | | | |
| Canine kidney cell | 98.2 ± 1.8 | 16.5 | 5 |
| line (MDCK) | | | |
| Mouse embryonic | 99.7 ± 0.6 | 33.5 | 20 |
| fibroblasts (MEF-1) | | | |

^{§}Mean ± S.D. of 3 independent experiments;
*Mean of 2 independent experiments.

### EXAMPLE 3

Cell Surface Expression of LRP is Essential for the Rapid Uptake of AL-Cy3ON.

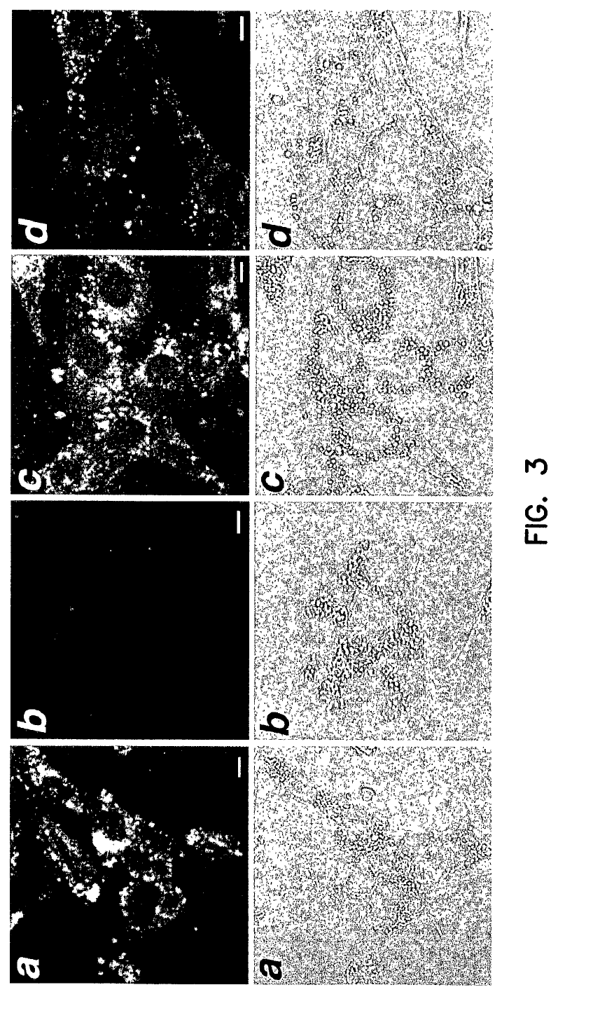

The endocytosis of AL-Cy3ONs in immortalized mouse embryonic fibroblast cell lines that expressed LRP (MEF-1) was compared to endocytosis of AL-Cy3ONs in immortalized mouse embryonic fibroblast cell lines that lacked the receptor (PEA-13). After a 1-h incubation, almost all MEF-1 cells displayed robust Cy3 fluorescence (FIG. 3a) in contrast to the faint signal seen in PEA-13 cells (FIG. 3b). Following a 3-h incubation, Cy3 fluorescence was visible in the PEA-13 cultures at lower intensity than that seen in the MEF-1 cells, indicating that liposomes were being taken up by the PEA-13 cells, albeit with very slow kinetics. In contrast to the MEF-1 cultures where all the cells examined had the Cy3 label, only 50-60% of the PEA-13 cells exhibited Cy3 fluorescence after 3 h (compare FIG. 3c with 3d).

To determine if endogenous proteins secreted by neurons could bind liposomes and act as intermediaries between liposomes and LRP, protein binding to liposomes after a 3-h incubation with neurons was measured. Incubations were carried out either in the absence or presence of 500 nM RAP to increase the possibility of protein-bound liposomes being recovered from the medium. The amount of protein detected by the CBQCA assay did not significantly differ between the untreated or RAP-treated controls and liposome-treated conditions (one-way ANOVA, p=0.5, Table 2). As a positive control, liposomes were incubated with poly-L-lysine (lysine/lipid phosphate charge ratios of 0.6 and 2) and 100% of the added polylysine was detected in the liposome pellet (not shown). As the amine moiety on the choline headgroup of DOPC was found to interact with the CBQCA dye, standard curves with BSA were constructed in solutions containing liposomes and the samples were diluted to minimize lipid interference. 10 nanograms of exogenously added BSA (not shown) was detected, indicating that within the limits of sensitivity of this assay, no endogenous proteins from cultured neurons bound liposomes.

### EXAMPLE 4

Anionic Liposome Endocytosis by LRP is Independent of HSPG and Does Not Alter Neuronal Calcium Contents.

Recent studies on cortical neurons and hippocampal slices have suggested a role for LRP in synaptic neurotransmission. Addition of activated a₂-M (a₂-M*) to cortical neurons caused a Ca²⁺ influx that was both spatially and temporally discrete. Only ligands that bind LRP at multiple sites were capable of eliciting this calcium response indicating that receptor dimerization was essential. To examine whether the endocytosis of anionic liposomes via LRP caused Ca²⁺ influx into neurons, neuronal calcium currents were studied during a continuous perfusion of liposomes labeled with N—Rh-DOPE.

Liposomes labeled with N—Rh-DOPE were prepared in a manner identical to that described in Example 1 for liposomes encapsulating Cy3ONs except that the lipid films contained 1-2.5 mole percent N—Rh-DOPE and the liposomes were prepared with buffer alone (i.e., without ONs).

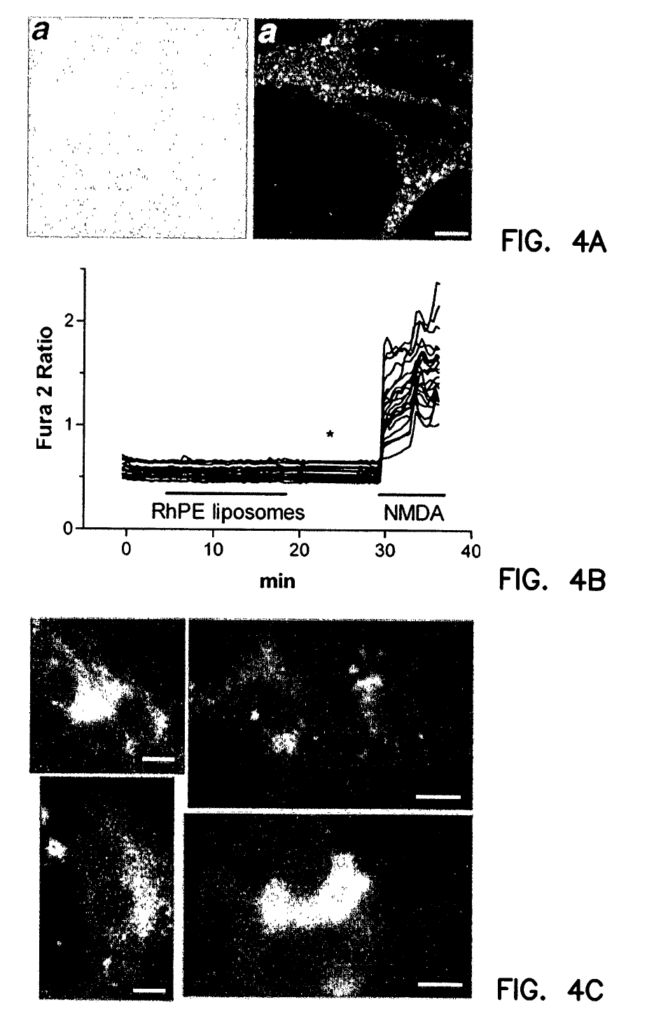

Anionic liposomes did not evoke a calcium response (FIG. 4b) although they were endocytosed as evidenced by rhodamine fluorescence in the neurons after liposomal perfusion (FIG. 4c).

TABLE 2
Analysis of endogenous protein binding to anionic liposomes.

| | Protein recovered |
|---|---|
| Condition | from liposome pellet (ng) |
| No treatment | 37.484 ± 11 |
| Anionic liposomes | 36.955 ± 10 |
| 500 nM RAP | 44.911 ± 4.9 |
| Anionic liposomes + | 40.85 ± 13 |
| 500 nM RAP | |

Protein amounts were determined by the CBQCA assay. Values are presented as Mean ± S.D., n = 3.

### EXAMPLE 5

### Protection of Neurons from Excitotoxic Death by p53 Antisense Oligonucleotides Delivered in Anionic Liposomes:

Experimental Procedures

Design and Synthesis of p53 ONs. The 18-mer p53 antisense ON used in this study (5′-CTGTGAATCCTCCATGAC-3′, SEQ ID NO:2) targets the translation initiation site of the rat p53 mRNA and is complementary to nucleotides 21 to 38 (GenBank accession number X13058 (Soussi et al., 1988)) with 50% GC content for optimal hybridization. Scrambled (5′-TCGATCTACGACTGACTC-3′, SEQ ID NO:3) and mismatch (5′-GAGTGAATGATCCATGGG-3′, SEQ ID NO:4) sequences were used as negative controls. The sequences had no similarity to other mammalian genes (BLAST search (Altschul et al., 1997)) and exhibited minimal self-complementarity (Vector NTI, Informax, Inc.). All ONs, synthesized as lyophilized powders by Midland Certified Reagent Company (Midlands, Tex.), were reconstituted in sterile, nuclease-free TE buffer (pH 7.4) and stored at −20° C. Concentrations of ONs in solution were routinely determined by absorbance measurements at 260 nm. Cy3-labeled oligonucleotides were synthesized by Integrated DNA Technologies, Coralville, Iowa.

Liposome Preparation. DOPC, DOPG, DOPS, DOPA, DOPE, DC-Chol and DOTAP were purchased from Avanti Polar Lipids, Alabaster, Ala. and stored at −20° C. as stock solutions of 2 mg/ml in chloroform. Anionic liposomes were prepared by a modification of the classic film hydration-extrusion procedure. Briefly, the lipid mixture was dried to a thin film under a stream of high-purity nitrogen and hydrated with a solution of ONs in 10 mM HEPES buffer (pH 7.4) with 5 mM NaCl (except when indicated otherwise) with intermittent heating and vortexing. After complete hydration, the suspension was transferred to a Liposofast™ miniextruder system (Avestin, Inc., Ottawa, Canada) and extruded through a series of polycarbonate membranes down to a pore size of 0.2 μm. Unencapsulated ONs were removed by loading the liposomes on a Sephadex G-50 column (7×0.5 cm, pre-equilibrated in hydration buffer) and centrifuging for 2 minutes at 180× g. Liposomes were eluted in the void volume and unencapsulated ONs were eluted in subsequent fractions. Purified liposomes were stored at 4° C. until use. Cationic DC-Chol/DOPE (1/1 molar ratio) liposomes were prepared in 10 mM HEPES buffer and extruded to 200 nm. The liposomes were diluted in 5% w/v glucose, complexed with ONs in various charge ratios and used immediately after complex formation. Commercial cationic liposomal transfection reagents TRANSFAST and TFX-20 were obtained from Promega (Madison, Wis.) and used according to the manufacturer's instructions.

Size Distribution Studies. Size analysis of liposomes was performed by quasi-elastic laser light scattering using a Nicomp Model 370 submicron particle sizer (Particle Sizing Systems, Santa Barbara, Calif.). At least one million particles were analyzed for each formulation and Gaussian or Nicomp distributions were chosen based on the chi-squared goodness of fit.

Assays for ON Encapsulation and Phospholipid Recovery. Aliquots (˜20 μl) of the liposome suspensions were diluted to 500 μl with distilled water and 500 μl of chloroform/methanol (1:1 v/v) was added to dissolve the liposomes. Aqueous and organic phases (containing the ONs and lipids, respectively) were separated by centrifugation at 1400× g for 10 minutes. This extraction procedure was repeated twice and organic solvents dissolved in the aqueous phase were removed by heating in a 95° C. water bath for 15 minutes. Known volumes of the extracted ONs were diluted to 100 μl with TE buffer and loaded onto a 96-well plate. An equal volume of a 1:200 dilution of OLIGREEN (Molecular Probes, Eugene, Oreg.) was added to the wells. The fluorescence increase upon binding of the dye to ON was measured using the FLUOSTAR microplate fluorometer (BMG Labtechnologies GmbH, Offenburg, Germany) with excitation and emission wavelengths of 480 and 535 nm. As OLIGREEN exhibits significant base selectivity, the amount of ON in the liposomes was calculated from standard curves generated with a known concentration of that particular ON in solution. For Cy3-labeled ONs, Cy3 fluorescence in the aqueous phase after extraction was measured directly at excitation and emission wavelengths of 544 and 590 nm. The amount of ONs present in the extracted aqueous phase, relative to the amount initially added to the lipid film, was used to calculate the percent ON encapsulated in the liposomes. Loss of phospholipid during liposome preparation was determined by adding chloroform and ammonium ferrothiocyanate (AFT) to the dried extracted organic phases. The mixture was vortexed to induce formation of the colored AFT/phospholipid complex that partitions into the chloroform phase (Stewart, 1980) and absorbance of the complex was measured at 475 nm (Beckman Instruments, Irvine, Calif.).

Hippocampal Cell Culture. Primary cultures of hippocampal neurons were prepared from neonatal rat pups (p1 or p2) as previously described (Dubinsky, 1993). Neurons were plated at a density of 60,000 cells/cm² onto polylysine-coated plastic 12-well plates or 100 mm dishes (Becton Dickinson, Franklin Lakes, N.J.) in Neurobasal medium with B27 supplements (Life technologies, Rockville, Md.) and 0.5 mM glutamine. Fluorodeoxyuridine (15 μg/ml) was added to the cultures 24 hours after plating to inhibit glial growth. Under these culture conditions, the survival and growth of non-neuronal cells was minimized. Cells were maintained at 37° C. in 95% air/5% CO₂ and were used between 6-8 days in vitro.

Neuroprotection Experiments. ONs (unencapsulated or in liposomes) were added to the culture medium at final concentrations of 0.1 to 5 μM, depending upon the experimental paradigm, for 3 hours and the neurons were then exposed to 50 μM glutamate. MK-801 and CNQX (final concentrations 20 μM each) were added 1-2 minutes before, and Pifithrin-α (final concentration 10 μM) 3 hours before, glutamate addition. Neuronal survival was assessed by counting viable cells in preselected fields based on trypan blue exclusion (Dubinsky et al., 1995) by an observer blinded to the treatments, 48 hours after glutamate exposure. The ratio of viable cells to the total number of neurons in the pre-selected fields was calculated for quantifying survival.

p53 Immunoprecipitation. Neurons (˜5 million cells/100 mm dish) were treated with 1 μM p53 antisense or scrambled ONs in anionic liposomes for three hours and exposed to glutamate for 15 hours. Cells were detached by scraping and sonicated in lysis buffer containing 0.1% SDS, 0.1% glycerol in 85 mM Tris HCl (pH 6.8) and protease inhibitor cocktail set III (Calbiochem, Cambridge, Mass.). After preclearing with Protein G-Agarose (IMMUNOPURE, Pierce, Rockford, Ill.), lysates were immunoprecipitated with the G59-12 monoclonal p53 antibody (2 μg/million cells, Pharmingen, San Diego, Calif.) and Protein G-Agarose. Immunoprecipitates and p53 positive control (Oncogene Research Products, Cambridge, Mass.) were resolved by 15% SDS-PAGE and proteins were transferred to an IMMOBILON-P membrane (Millipore, Bedford, Mass.). Blots were incubated with the CM1 rabbit polyclonal p53 antibody (1: 1000, Novocastra Laboratories, UK) and then probed with horseradish peroxidase-conjugated donkey anti-rabbit IgG (1:5000, Chemicon International, Inc., Temecula, Calif.). Detection was performed by enhanced chemiluminescence (ECL kit, Amersham Pharmacia Biotech, Arlington Heights, Ill.) and p53 levels were quantified using the Personal Densitometer SI and ImageQuant software (Molecular Dynamics, Sunnyvale, Calif.).

Statistical Analysis. Data were analyzed by one-way or two-way ANOVA with the Bonferroni post-test (GRAPHPAD PRISM, GraphPad Software, Inc., San Diego, Calif.).

Results

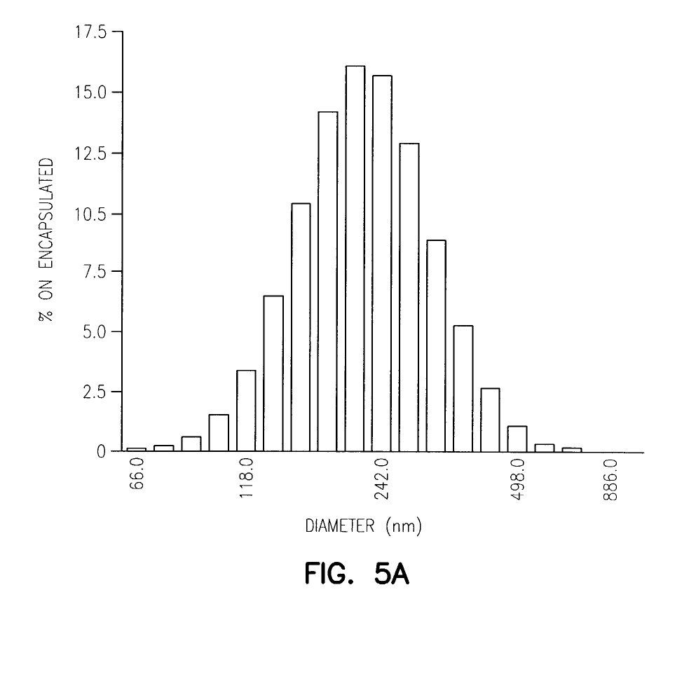

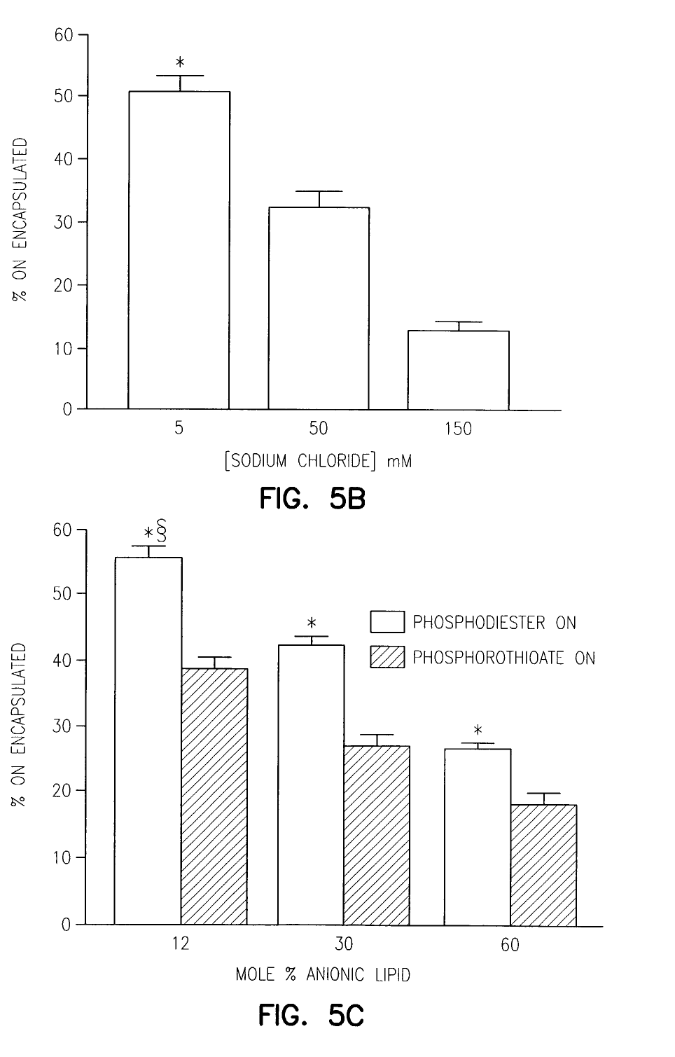

Characterization of Anionic Liposomes Encapsulating ONs. Liposomes composed of DOPC and 12 mole percent of one of the anionic lipids DOPG, DOPS, or DOPA were monodisperse suspensions with narrow Gaussian size distributions (FIG. 5A) and encapsulated 40-60% of the initial ON amount (Table 2) depending on the liposome composition. The amount of ON encapsulated in the liposomes was measured using the OLIGREEN dye which is highly specific for single-stranded nucleic acids, with a 1000-fold increase in dye fluorescence upon binding to a 20-mer ON (Singer et al., 1995). We also measured encapsulation of Cy3-labeled ONs in anionic liposomes by directly measuring Cy3 fluorescence and obtained identical results. Phospholipid content in the final preparations was 60-70% of the initial amount, reflecting losses during extrusion and purification by minicolumn centrifugation (Table 3).

TABLE 3
Physicochemical features of anionic liposomes encapsulating ONs

| | Mean Diameter ± | % ON | % phospholipid |
|---|---|---|---|
| Lipid | S.D. (nm) | encapsulated§ | recovered§ |
| DOPC/DOPG | 216.0 ± 75 | 56.8 ± 3.0 | 72.0 ± 6.1 |
| DOPC/DOPS | 242.3 ± 90 | 46.3 ± 10.7 | 69.3 ± 7.3 |
| DOPC/DOPA | 229.0 ± 92 | 44.4 ± 6.2 | 60.2 ± 8.7 |

Lipid films with 12 mole percent anionic lipid were hydrated with 75 nmoles of ONs in 500 μl of 10 mM HEPES buffer (pH 7.4) with 5 mM NaCl. Size distributions are of one representative sample.
§Mean ± S.D. of ≧5 independent experiments.

Ionic Strength, Anionic Charge Density and Oligonucleotide Chemistry Influence Encapsulation. Anionic liposomes composed of DOPC with 12 mole percent DOPG (DOPC/DOPG liposomes) were prepared in 10 mM HEPES buffer (pH 7.4) with 5, 50 or 150 mM NaCl and ON encapsulation was measured. Increasing the ionic strength of the hydration buffer dramatically decreased ON encapsulation (FIG. 5B). As the buffer with 5 mM NaCl allowed for maximum ON encapsulation, this was used for all subsequent studies. To investigate the role of anionic charge density on encapsulation, we varied the mole percent of anionic lipid in liposomes. Again, increasing the anionic charge of the lipid bilayer decreased encapsulation (FIG. 5C). We also compared the encapsulation of phosphodiester ONs in anionic liposomes to that of phosphorothioate ONs, as a function of mole percent of DOPG. Phosphorothioate ONs were encapsulated to a lesser extent than phosphodiester ONs and this decreased further with increasing anionic lipid content (FIG. 5C).

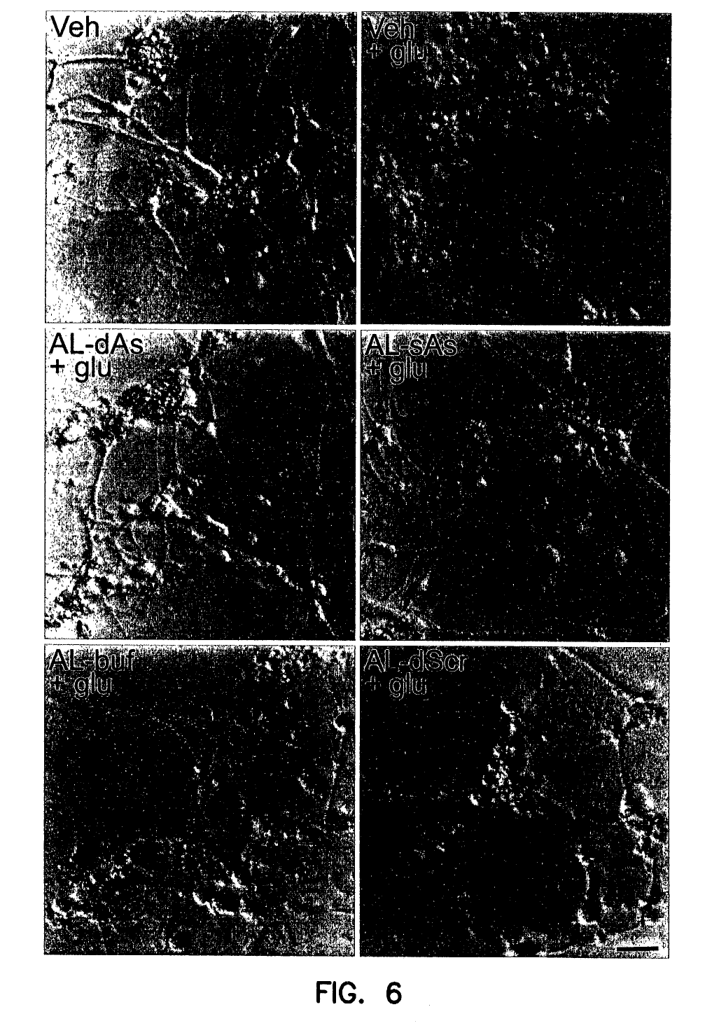

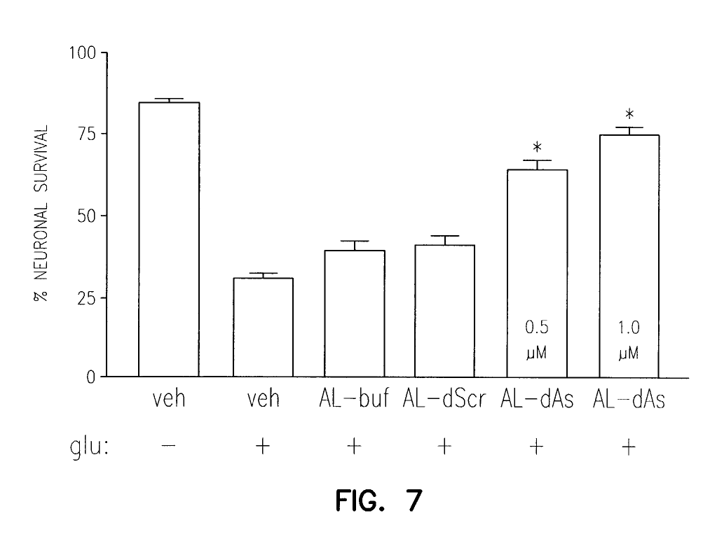

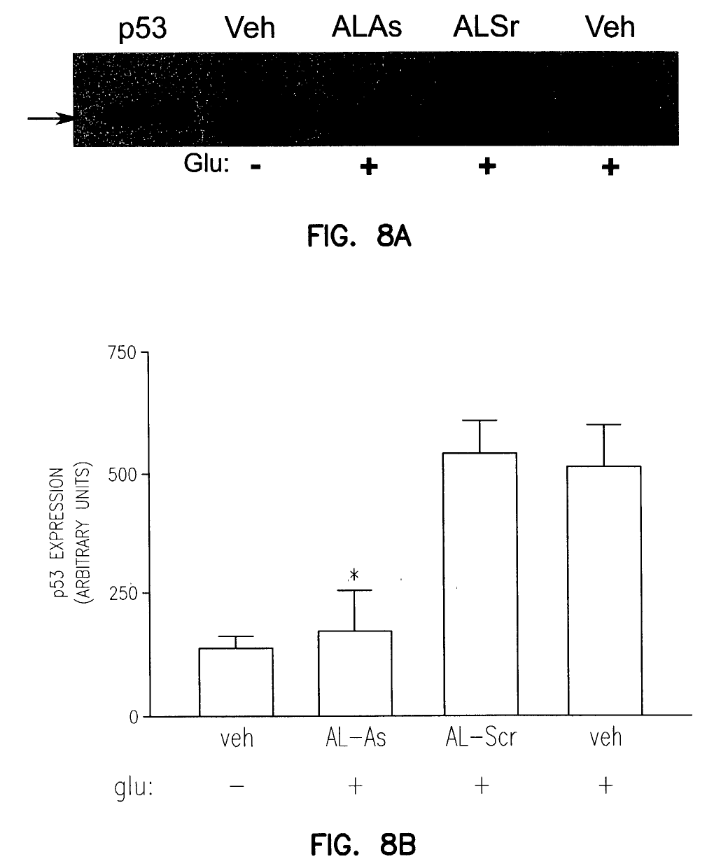

p53 Antisense ONs Delivered by Anionic Liposomes Elicit a Sequence-Specific Neuroprotective Effect. The ability of anionic liposomes to effectively deliver ONs to hippocampal neurons was evaluated in an in vitro model of glutamate toxicity. Neurons exposed to glutamate alone for 48 hours exhibited apoptotic features such as condensed, granular soma, neurite blebbing and fragmentation (FIG. 6, veh+glu). Note the extensive damage to neuronal processes and the cell bodies (soma) which appear blebbed. In contrast, neurons treated with 1 μM p53 AsONs delivered by anionic DOPC/DOPG liposomes retained intact processes and smooth soma after glutamate treatment, irrespective of the chemical nature of the ONs used (FIG. 6, AL-dAs and AL-sAs, anionic liposomes with phosphodiester and phosphorothioate p53 antisense ONs, respectively). Treatment with 0.5 and 1 μM p53 phosphodiester AsONs in DOPC/DOPG liposomes significantly increased the survival of neurons exposed to glutamate (FIG. 7, AL-dAs). This neuroprotection was sequence-specific as anionic liposomes with buffer alone or with 1 μM p53 scrambled ONs (FIGS. 6 and 7, AL-buf and AL-dScr, respectively) were ineffective. p53 protein levels in neurons treated with glutamate and ONs in DOPC/DOPG liposomes were determined by immunoprecipitation (FIGS. 8A and 8B). The results showed p53 antisense oligonucleotides protect neurons from excitotoxicity by down-regulation of the p53 protein. Neurons treated with p53 antisense ONs or scrambled ONs in anionic DOPC/DOPG liposomes for 3 hours followed by a 15 hour exposure to 50 μM glutamate were harvested for measurement of p53 protein levels by immunoprecipitation (FIG. 8). Exposure of hippocampal neurons to 50 μM glutamate for 15 hours increased p53 expression approximately 4-fold, relative to untreated neurons. Pretreatment of neurons with 1 μM p53 AsONs in DOPC/DOPG liposomes prevented the glutamate-induced increase in p53 protein levels by antisense-mediated down-regulation of p53 expression. In contrast, pretreatment with 1 μM scrambled oligonucleotides in anionic liposomes did not significantly alter the glutamate-induced increase in p53 expression, proving the specificity of p53 antisense sequence used in this study.

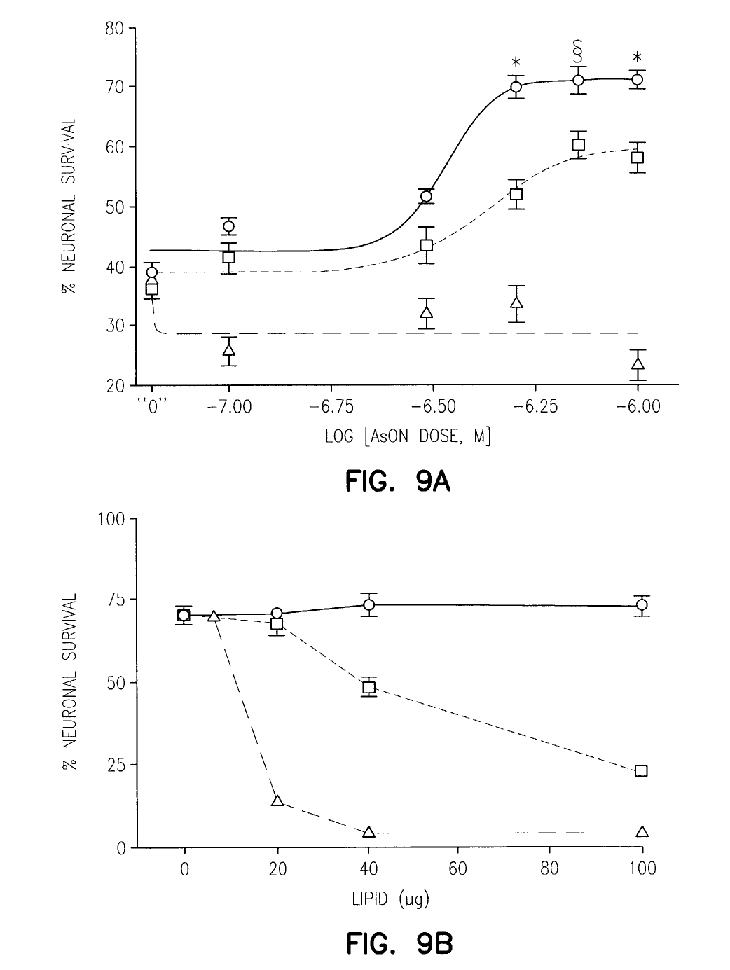

Liposome composition influences the extent of neuroprotection by p53 AsONs. The influence of liposomal lipids on the biological performance of the vector was studied by comparing the extent of neuroprotection by p53 AsONs delivered in DOPC/DOPG liposomes with that achieved by AsONs delivered (a) in liposomes where the anionic lipid DOPG was replaced by DOPS or (b) as complexes with cationic liposomes composed of DC-Chol/DOPE. DC-Chol was the model cationic lipid in our studies as it was best tolerated by neurons based on initial toxicity screens of DC-Chol, DOTAP and commercial transfection reagents TRANSFAST and TFX-20 (Table 4). p53 antisense ONs delivered by both anionic vectors caused a dose-dependent increase in neuronal survival after glutamate exposure while AsONs complexed with DC-Chol/DOPE were largely ineffective (FIG. 9A). However, greater neuroprotection was observed with p53 AsONs delivered by DOPC/DOPG liposomes compared to DOPC/DOPS liposomes at AsON doses of 0.5, 0.7 and 1 μM. To test whether the lipids themselves could exacerbate glutamate toxicity, we treated neurons with liposomes made solely of DOPG, DOPS or DC-Chol/DOPE (without AsONs), followed by exposure to a sub-maximal dose of glutamate (FIG. 9B). Addition of increasing amounts of DOPG did not appreciably change neuronal survival from the 71% seen after a 48 hour exposure to 10

TABLE 4
Toxicity screening of commercial cationic lipids

| Cationic Lipid | Lipid/ON (+/−) charge ratio | % Neuronal survival§ |
|---|---|---|
| DC-Chol | 2/1 | 17.3% |
| DC-Chol | 1/1 | 45.7% |
| DOTAP | 2/1 | ˜0% |
| DOTAP | 1/1 | 28.2% |
| TransFast ™ | 2/1 | ˜0% |
| Tfx-20 ™ | 3/1 | ˜0% |

Neuronal survival was assessed 8 hours after incubation with cationic lipid-ON complexes.
§percent survival compared to untreated controls, mean of two independent experiments.

μM glutamate. However, treatment with 40 μg DOPS (equivalent to the amount present in liposomes for a final ON concentration of 1 μM) decreased neuronal survival to 48%. Neurons were treated with amounts of cationic lipid required to complex 1 μM ONs in +/−charge ratios (μmole lipid/μmole ON) of 1/2, 1.6/1, 3.2/1 and 8/1 (6.25, 20, 40 and 100 μg DC-Chol, respectively). Only those neurons treated with 6.25 μg DC-Chol, i.e., where the complex would have a net-negative charge, survived 48 hours post-glutamate. Amounts of DC-Chol where the complex would be near-neutral or have a net positive charge caused extensive neuronal loss.

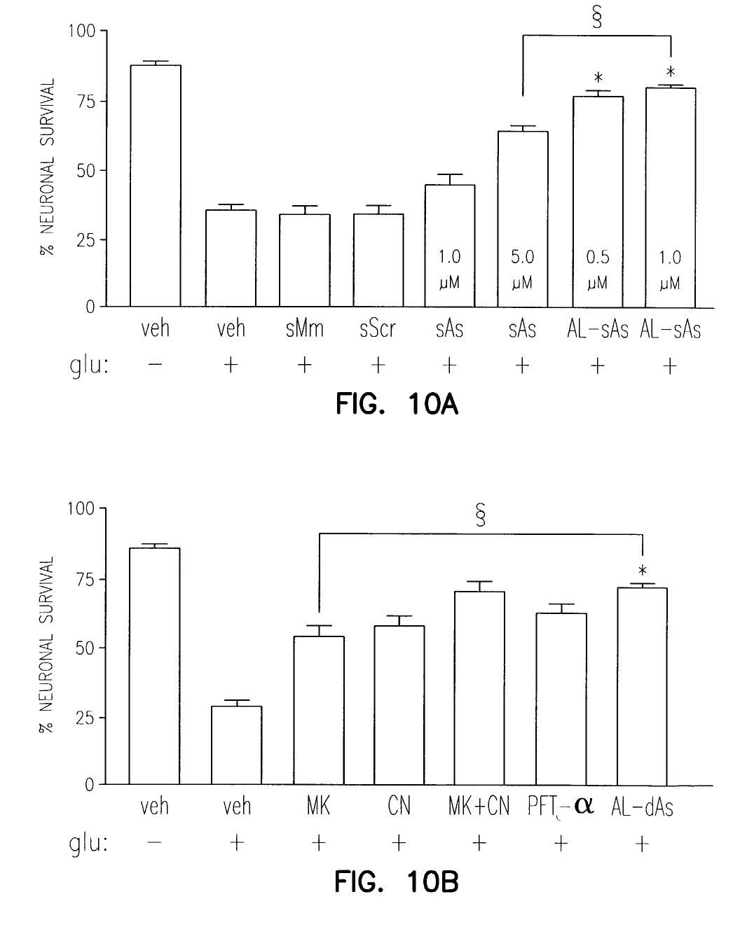

Anionic Liposomal Delivery of p53 Phosphorothioate AsONs Potentiates Antisense-Mediated Neuroprotection. Neuroprotection by p53 AsONs is potentiated when delivered by anionic DOPC/DOPG liposomes and is also comparable to that by glutamate receptor antagonists and p53 inhibitors (FIG. 10). While p53 phosphodiester AsONs were not neuroprotective when delivered “free” i.e., without encapsulation in anionic liposomes, free p53 phosphorothioate AsONs, at a dose of 5 μM, significantly increased neuronal survival (FIG. 10A, sAs) compared to neurons treated with glutamate alone. Phosphorothioate AsONs when delivered via DOPC/DOPG liposomes (FIG. 10A, AL-sAs) provided significantly more neuroprotection at concentrations of 0.5 and 1 μM than 5 μM free sAs. Neither phosphorothioate p53 scrambled ONs nor a sequence with 6 mismatches to p53 AsON were neuroprotective (FIG. 10A, sScr and sMm, respectively, 5 JIM each). Neuronal survival was also not increased by 1 μM phosphorothioate scrambled ON in anionic liposomes (data not shown). Phosphorothioate ONs at concentrations greater than 5 μM caused neurons to detach from the culture substrate within 12 hours of exposure and were not tested.

Neuroprotection by p53 ASONs Delivered by Anionic Liposomes is Comparable to that by the p53 Inhibitor, Pifithrin-α (PFT-α) and Glutamate Receptor Antagonists. PFT-α is a chemical inhibitor of p53 that was shown to protect cells from p53-induced apoptosis caused by genotoxic stress (Komarov et al., 1999). Antagonists to the NMDA and AMPA glutamate receptors, MK-801 and CNQX, 20 μM each, used individually or together, and 10 μM PFT-α significantly increased the survival of glutamate-treated neurons (FIG. 10B, MK, CN, MK+CN and PFT-α). In our hippocampal cultures, concentrations of PFT-α greater than 10 μM (20-100 μM) were toxic while lower concentrations (0.5-7 μM) were not protective. Neuroprotection afforded by 1 μM p53 AsON in DOPC/DOPG liposomes (FIG. 10B, AL-dAs) was greater than that by either MK-801 or CNQX, or PFT-α and comparable to that by MK-801+CNQX.

Discussion

Anionic liposomes are considered to be inefficient ON delivery vectors, primarily because of poor ON encapsulation reported previously (Zelphati and Szoka, 1996). Greater than 20 mole % anionic lipid in the bilayer decreased lipid-nucleic acid interactions, which might explain the low ON encapsulation in liposomes with 30 and 60 mole % anionic lipid. The lower encapsulation of phosphorothioate ONs can be attributed to increased repulsion between anionic lipid and the sulfur atom of phosphorothioate ONs compared to the oxygen atom of phosphodiester ONs.

The pattern of neuronal loss that occurs following excitotoxicity is an apoptotic-necrotic continuum, depending on mitochondrial function and the severity of the insult. In the present study, sequence-specific down regulation of p53 and concomitant neuroprotection was achieved by p53 AsONs delivered in anionic DOPC/DOPG liposomes. Moreover, the increase in neuronal survival due to p53 AsONs was comparable to glutamate receptor antagonists and the p53 inhibitor Pifithrin-α. The anionic lipid moiety (DOPG or DOPS) in the liposomes influenced the extent of neuroprotection achieved with p53 AsONs.

Cationic lipids, often used to transiently express reporter genes or downregulate specific proteins, have been successful with transformed cell lines where the cells are relatively healthy and no other manipulations (except addition or removal of cationic lipid-DNA complexes) are performed. However, conclusive evidence of the ability of cationic lipids to effectively deliver nucleic acids in a “rescue” paradigm is absent, due in large part to their inherent toxicity as seen in this and other studies (Hartmann et al., 1998; Kaech et al., 1996). Indeed, complexes of p53 AsONs with cationic DC-Chol/DOPE liposomes were unstable colloids and ineffective in rescuing glutamate-treated neurons. An exciting observation of our studies was the five- to ten-fold reduction of phosphorothioate AsON dose required to achieve maximal neuroprotection when delivered by anionic DOPC/DOPG liposomes. Thus, anionic liposomes not only increase the effectiveness of phosphorothioates but may also minimize their non-sequence-specific effects.

In conclusion, we have developed an anionic liposomal vector for oligonucleotides that overcomes the considerable limitations of cationic lipids. The unique properties of this vector allowed for efficient ON delivery to primary neurons and elicited a sensitive biological response. In addition to their therapeutic potential, these anionic liposomes may find application as a powerful tool for neurobiologists. Further elucidation of the biochemical and biophysical processes that underlie lipid-mediated DNA delivery explored in this study will greatly expand the possibilities for neuron-specific gene targeting.

### Example 6

### Anionic Liposomes Undergo Constitutive Receptor-Mediated Endocytosis in Hippocampal Neurons

The study described in this example was undertaken to answer the following questions: Are anionic liposomes internalized by a specific endocytic mechanism? If so, is the internalization receptor mediated? What is the intracellular itinerary of the internalized liposomes? We used Cy3-labeled oligonucleotides (Cy3ONs) encapsulated in anioniic liposomes and traced the uptake and intracellular route of these molecules in cultured rat hippocampal neurons using confocal microscopy. Each stage in the endocytic pathway was retarded by biochemically interfering with specific proteins to determine therole of that protein in the internalization of liposomes. We demonstrate that anionic liposomes were internalized in a fairly rapid manner via endocytosis triggered by binding to the low-density lipoprotein receptor-related protein (LRP).

Experimental Procedures

Oligonucleotide Design and Synthesis. An 18-mer oligonucleotide (5′-CTGTGAATCCTCCATGAC-3′, SEQ ID NO:2) that targets the translation initiation site of the rat p53 mRNA and is complementary to nucleotides 21 to 38 (GenBank accession number X13058 (Soussi et al., 1988)) was designed for this study. Oligonucleotides were synthesized and labeled at the 5′-end with Cy3 by Integrated DNA Technologies, Coralville, Iowa. The Cy3-labeled oligonucleotides were purified by reverse-phase HPLC to remove free dye. The oligonucleotides were reconstituted in sterile, nuclease-free Tris-EDTA buffer (pH 7.2) and stored at −20° C.

Liposome Preparation and Characterization. Dioleoyl phosphatidylcholine (DOPC), dioleoyl phosphatidylglycerol (DOPG), dioleoyl phosphatidylethanolamine (DOPE), dimethylaminoethane carbamoyl cholesterol (DC-Chol) and the headgroup labeled lipid Lissamine Rhodamine DOPE (N—Rh-DOPE) were purchased from Avanti Polar Lipids, Alabaster, Ala. and stored at −20° C. as stock solutions of 2 mg/ml in chloroform. Anionic liposomes encapsulating Cy3-oligonucleotides (AL-Cy3ONs) were prepared by a modification of the classic film hydration-extrusion procedure. Briefly, a lipid mixture of DOPC and DOPG was dried to a thin film under a stream of high-purity nitrogen, hydrated with a solution of Cy3ONs in 10 mM HEPES, 5 mM NaCl buffer (pH 7.4). After complete hydration, the suspension was transferred to a LIPOSOFAST miniextruder system (Avestin, Inc., Ottawa, Canada) and extruded through a series of polycarbonate membranes down to a pore size of 0.2 μm. Unencapsulated Cy3ONs were removed by minicolumn centrifugation using a Sephadex G-50 column. Liposomes were eluted in the void volume and unencapsulated Cy3ONs were eluted in subsequent fractions. Purified liposomes were stored at 4° C. until use. Cationic DC-Chol/DOPE (1/1 molar ratio) liposomes were prepared in 10 mM HEPES, 5 mM NaCl buffer and extruded to 200 nm. The liposomes were diluted in 5% w/v glucose, complexed with Cy3ONs in various charge ratios and used immediately after complex formation.

Size analysis of liposomes was performed by quasi-elastic laser light scattering using a Nicomp Model 370 submicron particle sizer (Particle Sizing Systems, Santa Barbara, Calif.). Liposomes were dissolved in equal volumes of water and chloroform/methanol and the resulting aqueous and organic phases were separated by centrifugation. Amount of Cy3ON encapsulated in liposomes was determined by measuring the fluorescence of the aqueous phase at standard Cy3 wavelengths (λex=552 nm and λem=570 nm) using the FLUOSTAR microplate fluorometer (BMG Labtechnologies GmbH, Offenburg, Germany).

Liposomes labeled with N—Rh-DOPE were prepared in a manner identical to that described for liposomes encapsulating Cy3ONs except that the lipid films contained 1-2.5 mole percent N—Rh-DOPE and the liposomes were prepared with buffer alone (i.e., without ONs).

Hippocampal Cell Culture. Primary cultures of hippocampal neurons were prepared from neonatal rat pups (P1 or P2) as previously described (Dubinsky, 1993). Neurons were plated onto 22-mm square glass coverslips coated with collagen and polylysine, at a density of 150,000 cells per coverslip. Neurons were cultured for 24-30 hours in minimum essential medium (MEM) with 10% NUSERUM (Collaborative Research) to increase attachment to the glass substrate, after which the growing medium was replaced with Neurobasal medium with B27 supplements (Life Technologies, Rockville, Md.). Fluorodeoxyuridine (15 μg/ml) was added to restrict the growth of non-neuronal cells. Cultures were maintained at 37° C. in a humidified atmosphere containing 5% CO₂ for 6-9 days before use.

Uptake of Cy3-ONs by hippocampal neurons. Neurons were incubated with 1 μM Cy3ONs either free, encapsulated in anionic liposomes or as complexes with cationic liposomes, for various time periods at 37° C. Uninternalized ONs, liposomes or complexes were removed by several washes with L15 (Leibovitz's) medium. Cells were then fixed in 4% paraformaldehyde for 15 minutes at room temperature. For low temperature studies, neurons were incubated at 4° C. with AL-Cy3ONs for 10 minutes and then fixed. The cells were imaged immediately after fixation to avoid a potential redistribution of ONs (Grzanna et al., 1998).

Endocytosis Assays. The following agents were used to manipulate specific steps of the endocytic cycle: 0.45 M hyperosmolar sucrose (ICN biochemicals), 1 μM FK-506 (Calbiochem), and 100 nM wortmannin, 5 μg/ml nocodazole, 10 μg/ml cytochalasin D, 100 μg/ml heparin and 100 μg/ml protamine sulfate, (all from Sigma-Aldrich). Receptor-associated protein (RAP) was a generous gift from Dr. Guojun Bu (Washington University, St. Louis, Mo.) and was used at a concentration of 500 nM (Bu et al., 1994). Cells were treated with the drugs or RAP for 10 minutes prior to incubation with AL-Cy3ONs for 30 minutes at 37° C. and fixed for imaging.

Colocalization of Lipids and Oligonucleotides with Transferrin and Dextran. Intracellular fates of endocytosed liposomes and ONs were determined by comparing their intracellular distributions with that of Oregon Green 488-Transferrin (OG-Tf) and Alexa488-dextran (Molecular Probes, Eugene, Oreg.) used as markers for the recycling and lysosomal compartments, respectively. Neurons were incubated with AL-Cy3ONs and either 100 μg/ml OG-Tf, for 30 minutes, 1 hour or 3 hours; or with 1 mg/ml Alexa488-dextran for 3 hours. For the lipid transport experiments, neurons were incubated with liposomes labeled with N—Rh-DOPE and either OG-TF, for 30 minutes, 1 hour, or 3 hours; or with 1 mg/ml Alexa-dextran for 3 hours. In another set of experiments, neurons were incubated with AL-Cy3ONs for 30 minutes, rinsed and chased with 1 mg/ml Alexa 488 dextran for 3 hours. Cells were rinsed and fixed for imaging as described above.

Fluorescence Resonance Energy Transfer Assays. To ensure that the headgroup-labeled lipid N—Rh-DOPE does not undergo spontaneous inter-bilayer transfer, liposome aggregation and fusion were monitored by fluorescence resonance energy transfer (FRET) using a single probe dilution assay. Liposomes labeled with 2.5 mole percent N—Rh-DOPE were mixed with a 10-fold excess of unlabeled liposomes in Neurobasal medium. Lissamine rhodamine undergoes concentration-dependent self-quenching when present in bilayers at concentrations greater than 1 mole percent (MacDonald, 1990). Liposome aggregation was induced by the addition of 25 mM calcium chloride and fusion was induced by 1% TRITON X-100 (Struck et al., 1981). The fluorescence increase of rhodamine as a result of dilution due to liposome fusion was continuously monitored over a period of 14 minutes with a Hitachi F-2000 fluorescence spectrophotometer at excitation and emission wavelengths of 550 nm and 590 nm, respectively.

Confocal Microscopy and Image Analysis. Imaging was performed on a Leica TCS 4D confocal microscope (Deerfield, Ill.) equipped with a Mercury/Xenon lamp and argon/krypton laser. Cells were excited using the 488 nm laser line to detect Oregon Green 488-Transferrin and Alexa488-Dextran, and the emitted fluorescence was collected using a 515 long-pass filter. The 568 nm laser line was used to excite Cy3 and N—Rh-DOPE (LP590 emission). Cells were imaged at a plane midway between the substrate-attached plasma membrane and the top of the cell, such that neuronal nuclei were clearly identifiable. In some cases, the entire volume of the cell was scanned in 0.5 μm increments. Optimal images were obtained by averaging 16 images in the line-scan mode at the same fixed gains for all experiments. All fluorescent images presented in figures were equally contrast enhanced using ADOBE PHOTOSHOP (Adobe, Mountain View, Calif.).

All image analysis was performed using the METAMORPH Imaging System software (Universal Imaging, Downington, Pa.). The cell outlines for each set of double-labeled fields were traced out manually in the corresponding differential interference contrast image and then used to mask the fluorescence images. In each cell, the total fluorescence intensity was measured and the percent of Cy3 label or rhodamine label that colocalized with transferrin or dextran was calculated.

Results

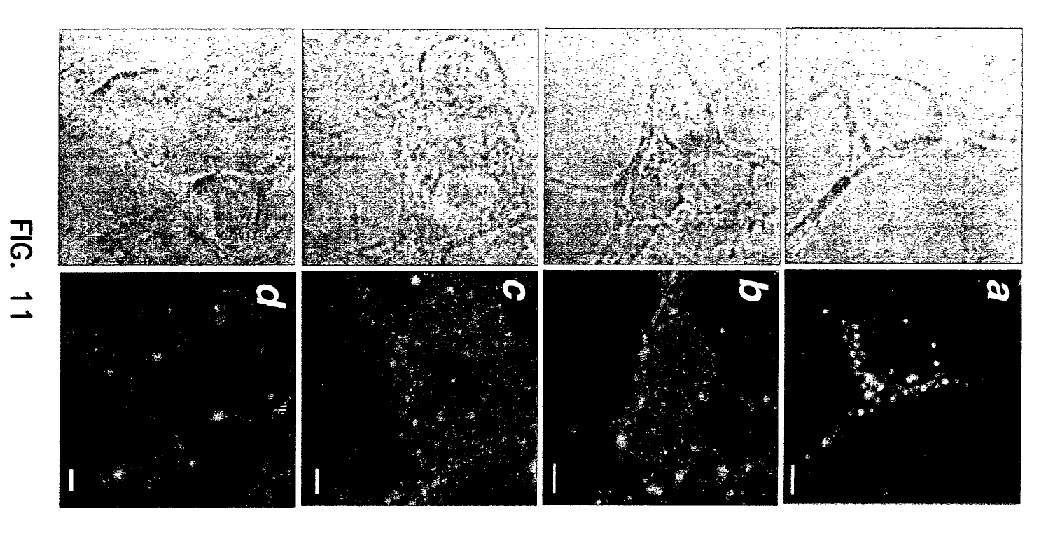

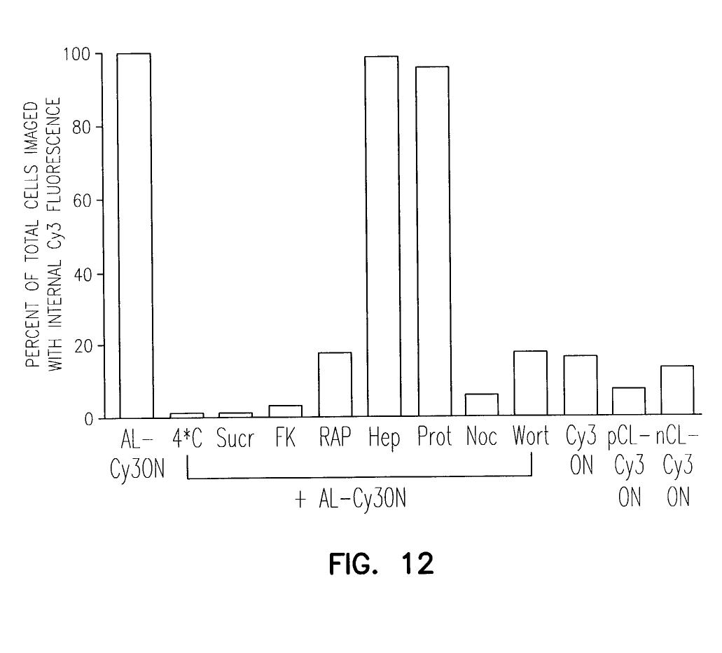

Neuronal uptake of anionic liposomes occurs by endocytosis. Incubation of hippocampal neurons with anionic liposomes containing 2 μM Cy3ONs for 30 minutes at 37° C. resulted in the localization of the labeled oligonucleotides in vesicular cytoplasmic structures but not in the nucleus (FIG. 11a). After a one-hour incubation, diffuse Cy3 fluorescence was observed in the nucleus (FIG. 11b). The intensity of the diffuse nuclear label increased after three hours and portions of the cytoplasm often contained uniform Cy3 fluorescence in addition to the punctate label (FIG. 11c). Virtually all the neurons imaged exhibited Cy3 fluorescence 30 minutes after incubation with anionic liposomes. Uptake of anionic liposomes was greatly reduced at 4° C. with Cy3 fluorescence seen only at the cell surface, indicative of binding of liposomes to the plasma membrane but no internalization (FIG. 11d). The time- and temperature-dependent uptake of anionic liposomes containing Cy3-oligonucleotides suggests that internalization of AL-Cy3ON by neurons occurs by an endocytic pathway, and is possibly receptor-mediated. The incidence of neurons with intracellular Cy3 fluorescence following various experimental manipulations is reported in FIG. 12.

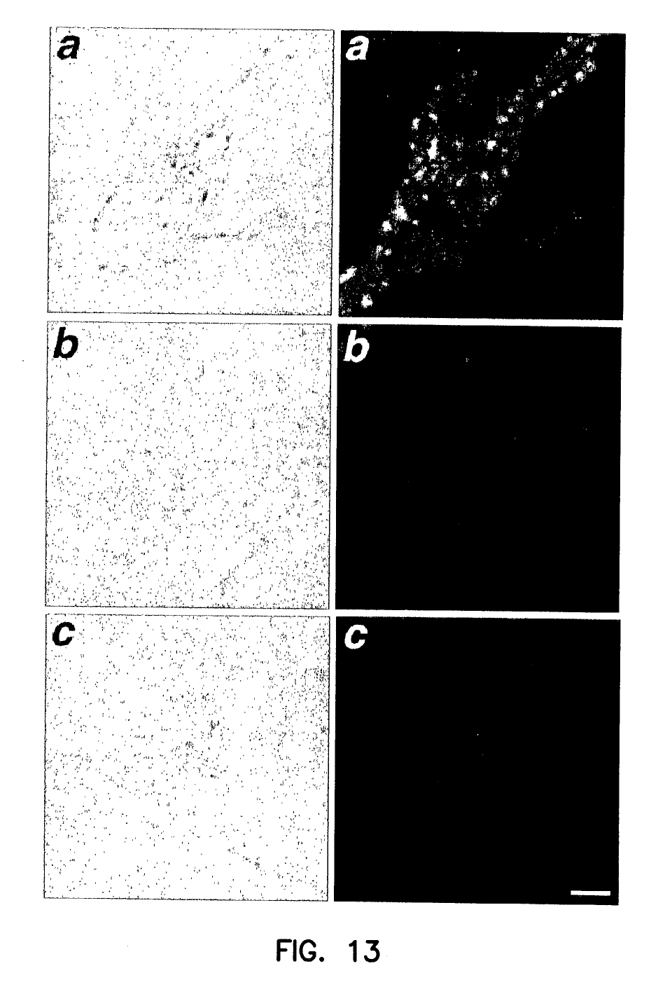

Intact clathrin lattices and functional dynamin are important for liposome internalization. Endocytosis, mediated by cell-surface receptors concentrated in clathrin-coated pits, is a major pathway for the internalization of macromolecules by cells. To determine if clathrin-coated pits are involved in the uptake of anionic liposomes, hyperosmolar sucrose was used to disrupt clathrin assemblies (Hansen et al., 1993; Oka et al., 1989). Pretreatment of neurons with 0.45M sucrose for 10 min completely prevented internalization of anionic liposomes containing Cy3-oligonucleotides (FIG. 13b) compared to cells treated with AL-Cy3ONs alone for 30 min (FIG. 13a). These results confirmed that neuronal uptake of liposomes was indeed achieved by a receptor-mediated endocytic mechanism as hyperosmolarity is known to inhibit receptor-mediated endocytosis, but not non-specific fluid phase endocytosis (Cupers et al., 1994; Oka et al., 1989).

Clathrin-mediated endocytosis plays a critical role in synaptic vesicle recycling at nerve terminals involving accessory proteins such as the guanosine triphosphatase dynamin, amphiphysin and synaptojanin (Brodin et al., 2000). For dynamin and amphiphysin to interact with each other and the lipid bilayer, they must be dephosphorylated by the Ca²⁺/calmodulin-dependent phosphatase, calcineurin (Bauerfeind et al., 1997; Lai et al., 2000; Powell et al., 2000). FK506 (Tacrolimus), an inhibitor of calcineurin, was used to study the role of dynamin in the internalization of anionic liposomes. Incubation of neurons with 1 μM FK506 for 10 minutes prior to the addition of AL-Cy3ONs significantly decreased liposome endocytosis (FIG. 13c), supporting the view that clathrin-dependent endocytosis is a major pathway for liposome internalization by neurons.

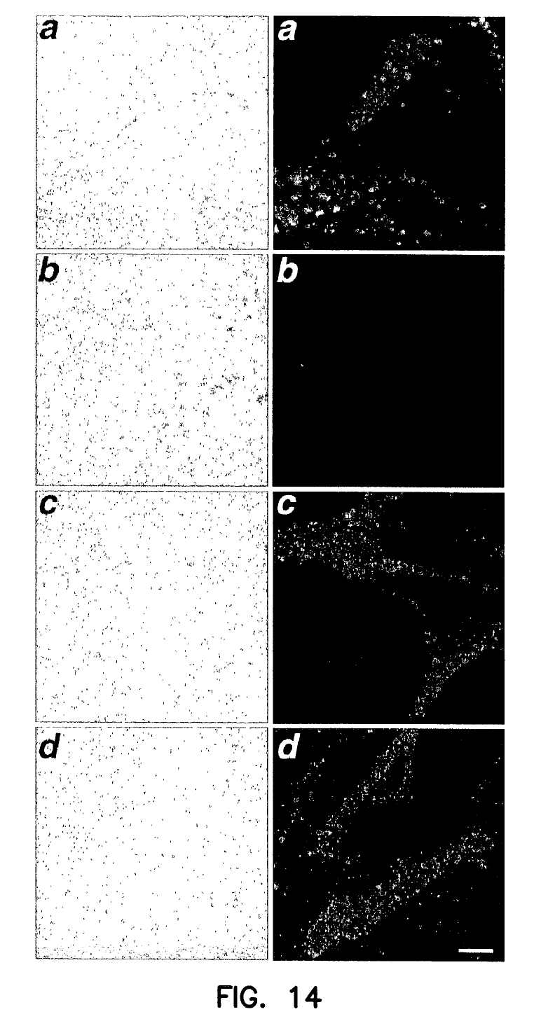

Liposome uptake occurs via LRP and does not involve heparan sulfate proteoglycans. The endocytic receptor low-density lipoprotein-related protein (LRP), which belongs of the LDL receptor gene family, is highly expressed in the mammalian central nervous system and has been implicated in the endocytosis of several unrelated ligands (Brown et al., 1997a). A major function of lipoprotein receptors is the regulation of cellular lipid uptake, membrane synthesis and metabolism (Willnow et al., 1999). To determine if LRP is involved in the endocytosis of anionic liposomes, we blocked LRP using the LRP receptor-associated protein (RAP) (Bu et al., 1994). The 39 kDa RAP is a potent inhibitor of all known ligand interactions of LRP. When neurons were incubated with AL-Cy3ONs in the presence of 500 nM RAP, both binding and internalization of anionic liposomes was inhibited. Note the complete absence of Cy3 fluorescence either on the cell surface or within the RAP-treated neurons (FIG. 14b) compared to those treated with AL-Cy3ONs alone (FIG. 14a).

Several LRP ligands, including (a₂-macroglobulin (a₂-M), apolipoprotein E (apoE), thrombospondin 1 (TSP1) and HIV Tat protein bind heparIn sulfate proteoglycans (HSPGs) on the cell surface prior to being internalized by LRP. In fact, RAP does not inhibit binding of TSP1 and HIV Tat to the plasma membrane but inhibits internalization and subsequent degradation of these ligands. On the other hand, LRP can also bind and internalize tissue factor pathway inhibitor (TFPI) in a manner independent from HSPGs (Warshawsky et al., 1996). To study whether HPSGs are necessary for liposome endocytosis by LRP, we incubated neurons with AL-Cy3ONs along with 100 μg/ml each of heparin or protamine sulfate. Heparin is a specific inhibitor of HSPG and protamine competes with LRP ligands for HSPG binding sites (Narita et al., 1995). Neither heparin nor protamine altered the level of Cy3 fluorescence within neurons after 30 minutes of incubation, indicating that LRP mediates liposome endocytosis in a manner that is independent of HSPGs (FIG. 14, c and d). These data show endocytosis of anionic liposomes is mediated by LRP, independent of heparin sulfate proteoglycans.

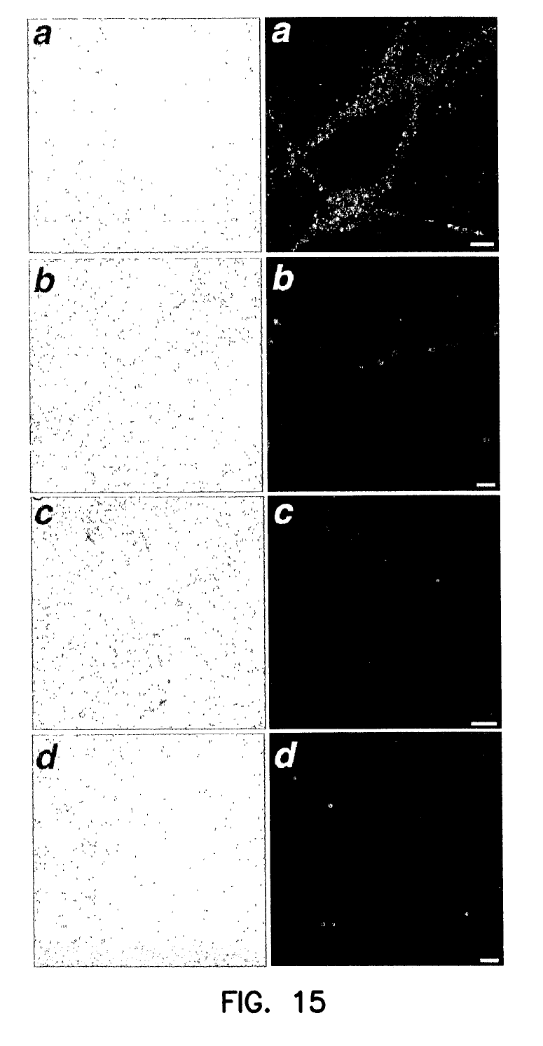

Transport of endocytosed anionic liposomes is associated with the cytoskeleton. Microtubule-dependent movement is a predominant means of axonal and dendritic transport, and depolymerization of microtubules (MT) inhibits both protein and phospholipid transport from the cell soma to the axons and dendrites (de Hoop and Dotti, 1993; Zakharenko and Popov, 1998). To determine if intracellular trafficking of AL-Cy3ONs requires an intact MT network, we used nocodazole to depolymerize microtubules in hippocampal neurons. When neurons were incubated with AL-Cy3ONs in the presence of 5 μg/ml nocodazole, Cy3 label was found only at the edges of the cell and on the plasma membrane. This indicated that although liposomes bound to the neuronal cell surface and may have been internalized, intracellular transport was inhibited (FIG. 15b compared to 15a).

Actin has been found to play a variable role in receptor-mediated endocytosis in different cell types (Freedman et al., 1999; Lamaze et al., 1997). Although it is widely accepted that an actin-based framework is important for the organization of clathrin-coated pits at the cell surface, the need for actin in receptor-mediated endocytosis is still under investigation (Lamaze et al., 1997). We studied the involvement of the actin cytoskeleton in liposome endocytosis using cytochalasin D to depolymerize actin filaments. In contrast to nocodazole-treated neurons, where Cy3 label was detected on the surface and at the rim of the cell, no Cy3 fluorescence was detected in neurons incubated with AL-Cy3ONs in the presence of cytochalasin D (FIG. 15c). This indicated that a 10 minute preincubation with cytochalasin D disrupted the organization of the clathrin-coated pits in neurons, and prevented both binding and internalization of the liposomes.

Intracellular trafficking of anionic liposomes depends on phosphatidylinositol 3-kinase (PI 3-K) activity. Activation of the PI 3-kinase family of lipid kinases leads to the generation of phosphoinositol-3,4-biphosphate and phosphatidylinositol-3,4,5-triphosphate which are involved in the rearrangement of cytoskeletal proteins, vesicle sorting and receptor recycling during endocytosis (Martin, 1997). Specific inhibitors of PI 3-kinase such as wortmannin have been widely used to study the potential sites of PI 3-K function in the endocytic pathway (Martys et al., 1996). To determine if PI 3-kinase activity is necessary for neuronal endocytosis of anionic liposomes, neurons were incubated with 100 nM wortmannin for either 10 or 20 minutes prior to the addition of AL-Cy3ONs. A low-level of cell-associated Cy3 fluorescence was observed in neurons pretreated with wortmannin for 10 minutes (FIG. 15d) and increasing the exposure time to wortmannin not only abolished the internalization of liposomes but also caused formation of vacuoles associated with the plasma membrane (data not shown). Previous studies have also documented a temporal correlation between exposure to wortmannin and drastic changes in organelle morphology (Shpetner et al., 1996). These data show an intact cytoskeleton and PI 3-kinase activity are important for AL-Cy3ON endocytosis.

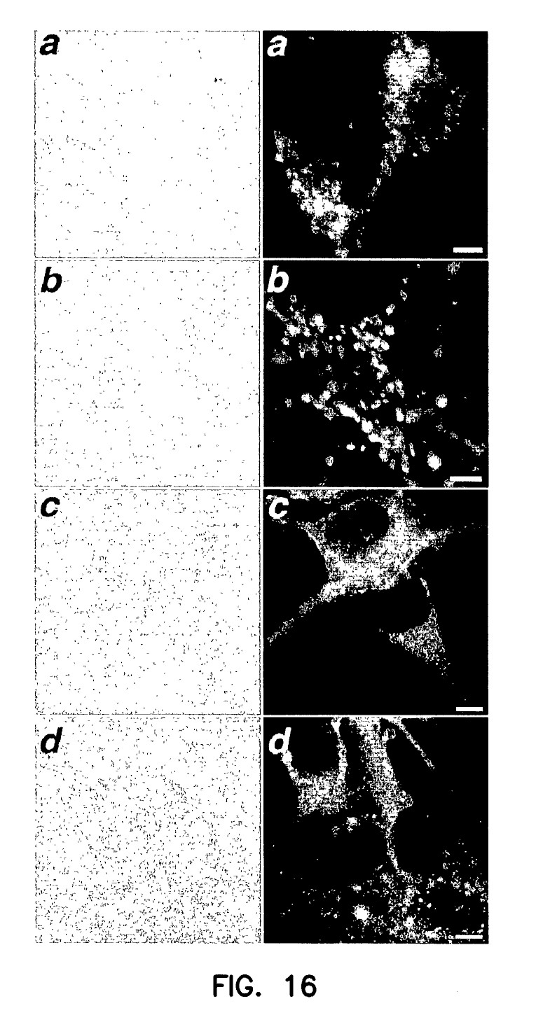

Cytoplasmic Cy3-ONs do not significantly colocalize with organelles containing transferrin or dextran. To determine the identity of the vesicular structures containing the Cy3 label, we incubated neurons with AL-CyONs along with either Oregon Green 488-transferrin (a marker for early and recycling endosomes) or Alexa 488-dextran (a marker for late endosome and lysosomes) for different time periods (FIG. 16, a and b, Table 5). After 30 minutes of co-incubation, 15% of the total intracellular Cy3 label was present in the same organelles as transferrin. The proportion of total Cy3 that colocalized with transferrin did not increase beyond 25% even after three hours of incubation. Only 20% of the total cell-associated Cy3 was present in compartments containing dextran. The lack of significant colocalization between Cy3ONs and transferrin indicates that Cy3ONs do not undergo recycling, or that recycling does occur, but with kinetics that are far slower than that of transferrin. As only 20% of Cy3ONs were present in lysosomal compartments after 3 hours, it is likely that the bulk of the ONs delivered via the endocytosed anionic liposomes were freely available to the cell.

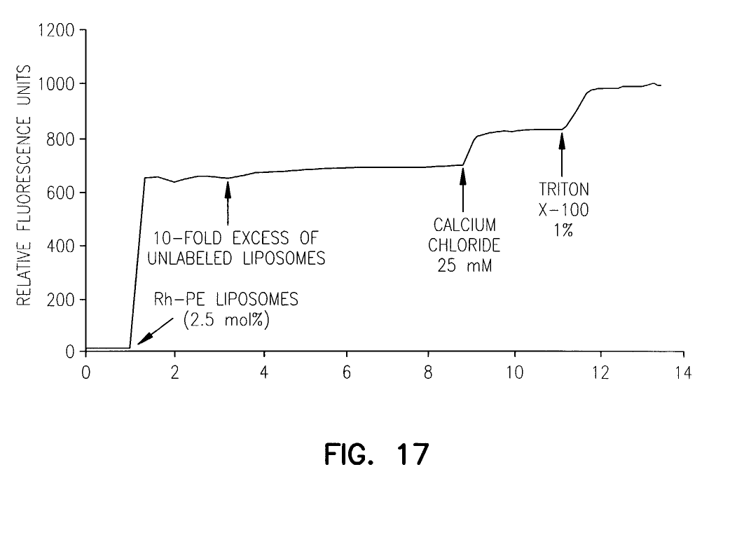

Liposomal lipids are preferentially sorted into recycling compartments. Recent evidence suggests that lipids endocytosed from the plasma membrane are sorted into either recycling or late endosomes based on the length and degree of unsaturation of their acyl chains (Mukherjee et al., 1999). To determine if the dioleoyl phospholipids (two 18-carbon acyl chains with one cis-double bond) used in our studies are similarly sorted, we fluorescently-tagged the liposomes with a headgroup-labeled lipid, N-Rh-DOPE. This lipid probe has been shown to be “non-exchangeable” i.e., it does not undergo spontaneous flip-flop between membrane leaflets and can therefore be expected to reliably label liposomal lipids during membrane trafficking after internalization (Willem et al., 1990). FRET measurements between unlabeled liposomes and liposomes labeled with N—Rh-DOPE confirmed this (FIG. 17). Mixing labeled liposomes with unlabeled ones did not relieve the self-quenching of rhodamine, which would have occurred if N—Rh-DOPE transferred between bilayers. There was an increase in rhodamine fluorescence only when probe dilution occurred due to calcium-induced liposome aggregation and TRITON X-100-induced bilayer fusion.

Once the non-exchangeable nature of the lipid label was established, neurons were incubated with N—Rh-DOPE liposomes and Oregon Green-488-transferrin for either 30 minutes or 1 h; or N—Rh-DOPE liposomes and Alexa-488-dextran for 3 h (FIG. 16, c and d). There was significant colocalization between transferrin and the liposomal lipids (Table 5). Approximately 50% of the internalized liposomal lipid colocalized with transferrin, suggesting that the “fluid” nature of the phospholipids that comprise the liposomes enhanced their sorting into recycling compartments.

Exploiting endocytosis for the delivery of macromolecules to neurons using anionic liposomes. Finally, neuronal uptake of anionic liposomes encapsulating oligonucleotides was compared with that of free oligonucleotides and oligonucleotides complexed with cationic lipids. These cationic complexes are thought to bind negatively charged cell membranes via electrostatic interactions and undergo non-specific endocytosis. We used cationic liposomes made of DC-Chol and DOPE and complexed them with Cy3ONs at two different charge ratios such that the resulting complexes would have either a net-positive or a net-negative

TABLE 5
Colocalization of Cy3-labeled oligonucleotides (Cy3ONs) or rhodamine-
labeled lipids (Rh-PE) with transferrin or dextran in hippocampal neurons.

| | Percent colocalization of Cy3ONs or | | |
|---|---|---|---|
| | N-Rh-DOPE with Transferrin or Dextran | | |
| Markers | 30 min | 1 h | 3 h |
| Cy3ON & | 14.86 ± 1 | 22.67 ± 1.7 | 24.62 ± 2 |
| Transferrin | (n = 43) | (n = 46) | (n = 46) |
| Cy3ON & | N.D. | N.D. | 20.13 ± 1.8 |
| Dextran | | | (n = 46) |
| N-Rh-DOPE & | 45.56 ± 2.2 | 51.88 ± 2.7 | N.D. |
| Transferrin | (n = 46) | (n = 43) | |
| N-Rh-DOPE & | N.D. | N.D. | 22.18 ± 1.5 |
| Dextran | | | (n = 43) |

Quantitation of the extent of colocalization of Cy3ONs or N-Rh-DOPE with markers for recycling endosomes (transferrin) and lysosomes (dextran) was performed as detailed in Experimental Procedures. n, number of neurons imaged per condition in three separate experiments; N.D., not determined.

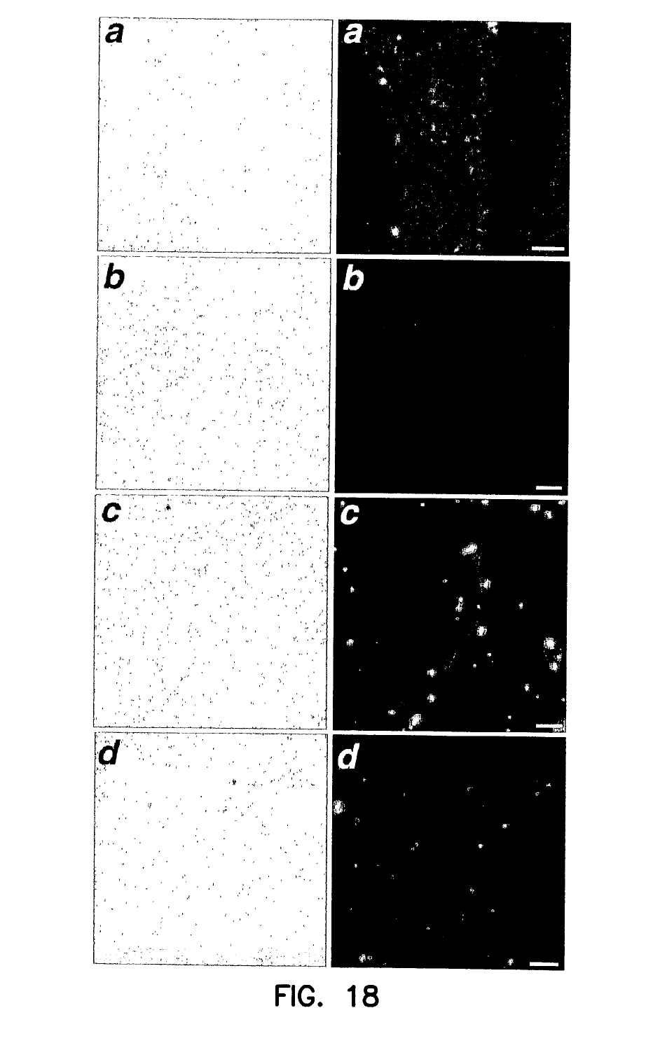

charge. Incubation of hippocampal neurons with net-positive cationic lipid-Cy3ON complexes or with 2 μM “free” Cy3ONs, i.e., without a delivery vector, for 30 minutes at 37° C. resulted in a low level of diffuse cellular fluorescence in only a small percent of cells (FIG. 18, b and c; FIG. 12) compared to cells treated with AL-Cy3ONs (FIG. 18a). Neurons incubated with net-negative cationic lipid-Cy3ON complexes exhibited bright fluorescence associated with the plasma membrane, with only sparse fluorescence observed inside occasional neurons (FIG. 18d). Studies have reported that cationic lipids exhibit significant cytotoxicity that can be directly correlated both to the cationic lipid concentration and time of exposure to the complexes (Kaech et al., 1996). In this respect, we have demonstrated that anionic liposomes are non-toxic and rapidly deliver oligonucleotides to neurons via a receptor-mediated endocytic pathway, thus providing an efficient method for enhancing the uptake of oligonucleotides and presumably, other macromolecules, into neurons.

Discussion

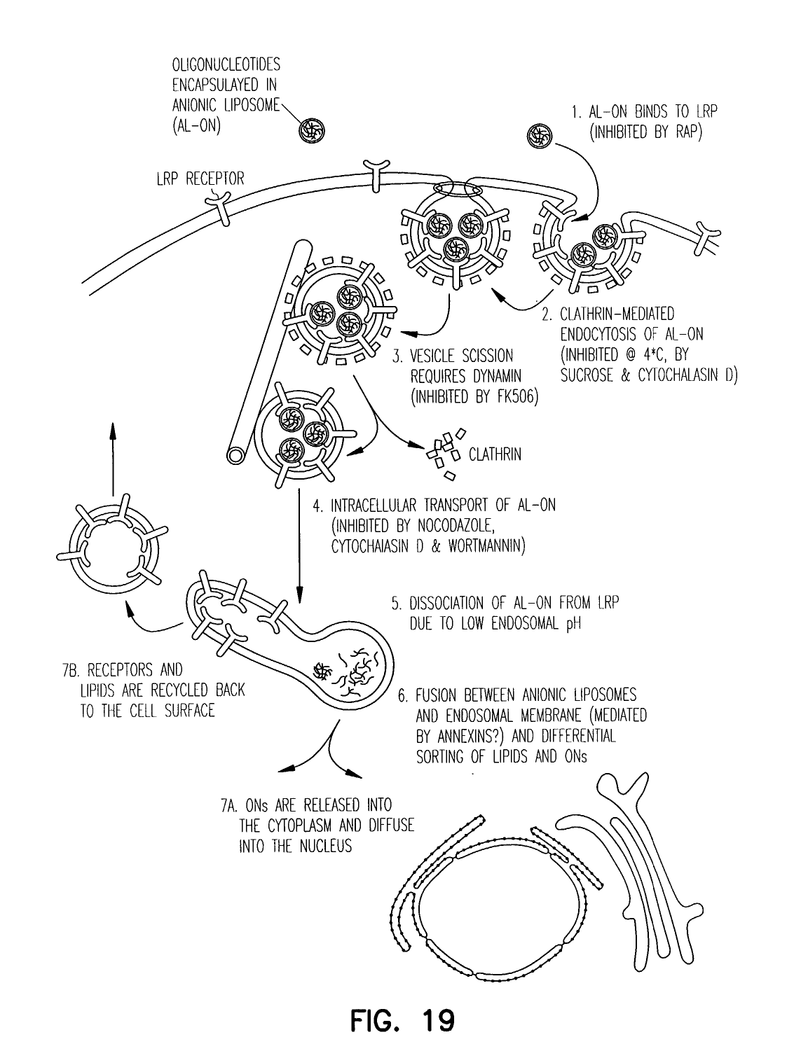

The proposed molecular mechanisms of internalization of anionic liposomes by hippocampal neurons involve normal components of constitutive clathrin-mediated endocytosis (FIG. 19). Recruitment of cell surface receptors into clathrin-coated pits and interaction of the receptor's cytoplasmic internalization signal with the clathrin adaptor protein AP2 are inhibited at 4° C. (Fire et at., 1997). Similarly, the time-dependent transport of Cy3ONs to the nucleus at 37° C. (FIG. 11, a-c) and inhibition of internalization of AL-Cy3ONs seen at 4° C. (FIG. 11d) indicated that liposomes are taken up by an energy-dependent process such as clathrin-mediated endocytosis. Hyperosmolarity interferes with endocytosis by breaking down clathrin assemblies, forming microcages and resulting in the random dispersal of receptors in the plasma membrane (Heuser and Anderson, 1989). Hyperosmolar sucrose inhibits the receptor-mediated endocytosis of transferrin (Bowen and Morgan, 1988), asialoglycoprotein (Oka et al., 1989) and low-density lipoprotein (Heuser and Anderson, 1989) but not fluid-phase endocytosis (Cupers et al., 1994; Oka et at., 1989). Exposure of neurons to hyperosmolar sucrose drastically decreased the internalization of AL-Cy3ONs, again implicating a process of clathrin-mediated endocytosis (FIG. 13b).

Dynamin, a cytosolic GTPase, is recruited to clathrin coated pits by amphiphysin which can simultaneously bind AP2 and dynamin through different domains. Stimulus-dependent dephosphorylation of dynamin and amphiphysin by calcineurin is essential for their assembly into a functional endocytic complex. Dynamin then self-assembles into tetramers that polymerize into ring-like structures around the neck of the coated pit, pinching it off from the plasma membrane resulting in the formation of a vesicle (Sweitzer and Hinshaw, 1998). In our studies, endocytosis of anionic liposomes by neurons was significantly reduced by treatment with FK506, indicative of dynamin's involvement in endocytosis (FIG. 13c). The time course of liposome endocytosis and the appearance of Cy3 label in the neuronal nucleus within one hour of incubation coupled with the requirement for clathrin and dynamin in anionic liposome uptake strongly indicate that a neuronal cell surface receptor might be responsible for the rapid internalization of liposomes.

The LDL receptor-related protein (LRP) is an endocytic receptor that is expressed in a spectrum of tissues (Moestrup et al., 1992), including the nervous system, with high expression seen in the cerebellum, cortex, hippocampus and brain stem (Bu et al., 1994). In cultured hippocampal neurons, LRP shows a polarized distribution and is restricted to the somatodendritic domain (Brown et al., 1997a). LRP is synthesized as a 600 kDa protein that undergoes proteolytic processing to form a heterodimer with a 515 kDa extracellular subunit noncovalently linked to an 85 kDa subunit that contains a single membrane-spanning domain and two NPXY internalization motifs (Herz et al., 1990). The 515 kDa subunit is the ligand binding region and LRP is known to bind at least 20 structurally and functionally distinct ligands such as apolipoproteins, protease-protease inhibitor complexes, pseudomonas exotoxin and most recently, the HIV tat protein (FitzGerald et at., 1995; Kounnas et al., 1995a).

Polymorphisms in apoe, α₂-M and LRP genes are known to affect the risk for late-onset Alzheimer's disease; and LRP, apoE and other LRP ligands localize to senile plaques (Hyman et al., 2000). The epsilon3 allele of apoE (apoE3, the most common isoform) but not the epsilon4 allele (apoE4, a risk factor for late-onset AD) enhances neurite outgrowth of cultured hippocampal neurons (Narita et al., 1997). RAP and anti-LRP antibodies inhibit apoE3-induced neurite growth and protect neurons from apoE4 toxicity, indicating that LRP mediates both actions. LRP is also involved in the endocytosis and lysosomal degradation of complexes of amyloid precursor protein (APP) with α₂-M, and may thus modify the generation of β-amyloid peptides (Kounnas et at., 1995b). The intracellular signaling functions of LRP are just being unveiled and may occur via its interactions with a heterotrimeric GTPase.

Given the well-defined role of LRP in lipid metabolism (Willnow, 1999; Willnow et al., 1994b), we investigated the involvement of LRP in anionic liposome endocytosis using RAP as an LRP inhibitor. RAP binds with high affinity to the heavy chain of LRP on multiple ligand binding domains and is thought to decrease the affinity of LRP for its ligands by inducing a conformational change in the receptor (Williams et al., 1992). Both the binding and endocytosis of anionic liposomes in hippocampal neurons were prevented by RAP, implicating LRP as the receptor involved in the uptake of anionic liposomes (FIG. 14b). Many LRP ligands first bind to cell surface HSPGs and are then transferred to LRP for internalization. The endocytosis of ligands like apoE, lipoprotein lipase and APP is inhibited when their binding to HPSG is prevented by heparin or protamine (Kounnas et al., 1995b; Tolar et al., 1997). The binding and endocytosis of anionic liposomes by neurons was not influenced by presence of heparin or protamine and therefore, occurred independent of HSPGs (FIG. 14, c and d).

The 515 kDa chain contains four separate ligand-binding domains, each of which is characterized by clusters of complement-type repeats and epidermal growth factor (EGF)-like repeats. In all, LRP has 31 ligand binding type repeats (compared to 7 in the LDL receptor), predicting that this protein has numerous ligand recognition sites, all of which may bind different ligands. Available evidence indicates that binding sites for protein ligands are largely restricted to clusters of complement-type repeats (Willnow et at., 1994a). Like the repeats of the LDL receptor, those of LRP also contain six cysteine residues that form three intradomain disulfide bonds. Further, each repeat contains a single Ca²⁺ ion trapped in an octahedral cage formed by four conserved acidic residues along with two carbonyl oxygens that stabilizes receptor structure (Brown et al., 1997b).

Many LRP ligands (apoE, HIV tat) have clusters of basic residues in their receptor-binding domains that are proposed to interact with the conserved acidic residues on the receptor (Mikhailenko et al., 1997; Rodenburg et al., 1998). Another school of thought suggests that hydrophobic interactions play a greater role because the acidic residues interact with calcium and would not be available for ligand binding. However, given the low sequence homology between the repeats (with the exception of the conserved cysteines and acidic residues) and the variety of ligands that LRP binds, it is safe to assume that no one common binding mode exists for this receptor.

In addition to endogenous protein ligands of LRP that have been extensively studied, LRP on rat mesangial cells was shown to bind a heparin-like anionic polymer of 4-hydroxyphenoxy acetic acid (Katz et at., 1997). In the present study, the complete inhibition of anionic liposome endocytosis by RAP suggests that anionic liposomes are capable of interacting with LRP and are thus internalized into neurons.

Once internalized, clathrin-coated endocytic vesicles move to the endosome along cytoskeletal structures. Depolymerization of microtubules in hippocampal neurons with nocodazole caused a total cessation of endosomal movement and decreased the apparent speed of the endosomes (Prekeris et al., 1999). Endosomal “storage” pools of cell surface receptors arise from constitutive endocytosis of unoccupied receptors (Ajioka and Kaplan, 1986), and insulin rapidly mobilized LRP from the intracellular pool to the cell surface (Descamps et at., 1993). Within 5 minutes of incubation at 37° C., ˜60% of the surface LRP was internalized and ˜50% of LRP recycled back to the plasma membrane 30-60 minutes after endocytosis (Ko et al., 1998). Thus, both the transport of internalized liposomes and the endosomal recycling of LRP to the cell surface would be expected to be inhibited by cytoskeletal disruption. In the presence of nocodazole, which depolymerizes microtubules or cytochalasin D, which depolymerizes actin filaments, endocytosis of anionic liposomes encapsulating Cy3ONs was decreased (FIG. 15, b and c). Cy3 label was clearly visible on the cell surface and immediately inside the cell in nocodazole-treated neurons, indicating that binding and internalization of liposomes occurred but further transport of the coated vesicles was inhibited. Neurons treated with cytochalasin D exhibited minimal surface binding and internalization of the anionic liposomes in agreement with reports that the cytochalasin D-sensitive step precedes the nocodazole-sensitive step in receptor-mediated endocytosis (Maples et at., 1997). While the role of actin in the intracellular transport of endocytic vesicles has been a matter of much debate (Lamaze et at., 1997), our results suggest that actin is important for LRP-mediated endocytosis probably due to its role in the maintenance of the structural organization of clathrin-coated pits (Gaidarov et al., 1999).

Phosphatidylinositol 3-kinase (PI 3-k) activity is necessary for numerous cellular functions including mitogenesis, differentiation, cytoskeletal regulation and vesicle trafficking (Martin, 1997). The P1 3-k inhibitor wortmannin inhibits early endosome fusion by regulating the activity of the small GTPase Rab5 (Li et al., 1995; Sonnichsen et al., 2000). Activated Rab5 and P1 3-phosphate generation are important for the binding of EEA1 (early endosomal antigen 1) to the endosomal membrane. EEAI in turn, directly interacts with the SNARE complex, thus forming a restricted fusion-competent domain on the early endosome (Pfeffer, 1999). Wortmannin not only interferes with the transport of endocytic vesicles that bud off from the plasma membrane but also halts receptor recycling. Increased surface presentation of LRP in response to insulin was almost completely inhibited by wortmannin (Ko et at., 2001). A role for P1 3-K activity was confirmed in our experiments where the endocytosis of anionic liposomes was dramatically reduced in the presence of wortmannin (FIG. 15d).

The identity of cytoplasmic compartments containing the Cy3-label was investigated using transferrin as a marker for early and recycling endosomes and dextran as a marker for late endosomes and lysosomes. The lack of extensive colocalization of the Cy3-label with either transferrin or dextran indicated that the majority of Cy3ONs were neither recycled nor subjected to appreciable, rapid lysosomal degradation within neurons (FIG. 16, a and b; Table 4). The poor colocalization between Cy3ONs and transferrin could also mean that ON recycling is much slower than that of transferrin, like glycosylphosphatidylinositol (GP 1)-anchored proteins that are recycled three times more slowly than transferrin (Mayor et al., 1998). The processing of liposomal lipids was also studied using rhodamine as a lipid label (FIG. 16, c and d; Table 4). Within 1 h of incubation, ˜50% of the internalized lipid was present in transferrin-containing compartments, in agreement with reports that acyl chain length and degree of unsaturation determine the compartments into which membrane lipids are sorted (Mukherjee et al., 1999). According to this model, lipids with long unsaturated chains or those with head group cross-sectional areas equal to or lesser than that of the acyl chains have either no curvature preference or partition into membranes with concave curvature, and are thus sorted into tubular recycling endosomes. The lipids used in our studies have two C18 acyl chains with one cis-double bond each (dioleoyl) and are known to preferentially partition into fluid lipid domains such as those of the tubulovesicular recycling endosomes. Lipids with saturated or trans-unsaturated chains preferentially partition into more rigid domains (Klausner and Kleinfeld, 1984) and most likely end up in lysosomes (Mukherjee et al., 1999).

Many LRP ligands (apoE, APP, TPA) undergo lysosomal degradation while a few, like the HIV tat protein, make their way into the cytoplasm and subsequently, to the nucleus. Nuclear localization of Cy3ONs was also observed in our experiments suggesting that ONs were capable of bypassing the endosomal/lysosomal pathway. The differential colocalization of ONs and liposomal lipid with transferrin indicates that between 30 min-1 h after liposome endocytosis, the intracellular paths of lipids and ONs diverge. Among the many proteins that are present on the lumenal face of early endosomes, those belonging to the annexin family (annexins I, IV and VI) bind anionic phospholipids in a calcium-dependent manner (Kobayashi et al., 1998). Annexin IV causes lateral segregation of phosphatidylglycerol in mixed bilayers of phosphatidylcholine (PC) and phosphatidylglycerol (PG) in the presence of physiological concentrations of Ca²⁺ (Junker and Creutz, 1993). More pertinently, liposome fusion induced by annexin I may be dependent upon the presence of PG in the bilayer (Koppenol et at., 1998). Given that PG is the anionic lipid component of the liposomes used in this study, it is tempting to speculate about the role of annexin in liposome fusion. After uncoupling of LRP from liposomes at the low pH in early endosomes, annexin IV may mediate destabilization and/or fusion of the liposome bilayer with that of the endosomal membrane. This would provide a conduit for ONs into the cytoplasm from where they can freely diffuse into the nucleus.

Neuronal uptake of “free” Cy3ONs, i.e., delivered without encapsulation in anionic liposomes, was very low compared with AL-Cy3ONs, and most cells showed a low level of diffuse fluorescence (FIG. 6, a and b). Recently, a 35 kDa protein found on the mitochondrial outer membrane called porin (also known as the voltage-dependent anion channel (VDAC)) was shown to translocate double stranded DNA across planar bilayers (Szabo et al., 1998). Porin is concentrated in caveolae-like domains on the plasma membrane of many cells, including neurons (Bathori et al., 1999). While porins present on the neuronal plasma membrane may well mediate the uptake of unencapsulated Cy3ONs, it certainty does not appear to be an efficient process. Cationic liposomes, widely used for DNA delivery to immortalized cells, are thought to be taken up by a nonspecific endocytic process after the complexes bind to the negatively charged cell membrane (Marcusson et al., 1998; Zelphati and F. C Szoka, 1996). The rate of endocytosis depends on the cell type and occurs relatively slowly. For instance, in COS and HeLa cells, only 5% of the cells took up the complexes after 30 minutes of incubation. Maximal uptake was achieved at 6 hours with ˜50% of the cells taking up the complex (Zabner et at., 1995). Neurons incubated with cationic lipid-Cy3ONs complexes with either a net-negative or net-positive charge exhibited hardly any intracerlular Cy3 fluorescence after 30 min (FIG. 18, c and d). This is in marked contrast to the uptake seen with anionic liposomes where almost 100% of the neurons showed Cy3 fluorescence after 30 minutes of incubation (FIG. 12).

The requirement of a net positive charge on cationic lipid-DNA complexes for efficient delivery and transfection is thought to depend on the cell type. For primary cells and in vivo applications, a net-negative charge on the complex has been found to be optimal (Schwartz et at., 1995). The low efficiency of DNA delivery by cationic lipids can be attributed to a greater lethality of cationic lipids to primary cells in general and neurons in particular (Kaech et at., 1996; Lakkaraju et at., 2001) and the post-mitotic nature of neurons.

A major drawback for the realization of genetic therapies has been the lack of a suitable vector for the effective delivery of DNA to cells. The relatively rapid endocytosis of anionic liposomes in post-mitotic cells like neurons and the transport of liposomal cargo to the cytoplasm and nucleus indicate that anionic liposomes are capable of overcoming several obstacles to successful gene delivery. Additionally, the widespread expression of LRP (Moestrup et al., 1992) should enable anionic liposomes to deliver nucleic acids and, possibly, proteins, to a broad spectrum of tissues.

### DOCUMENTS REFERED TO

Ajioka, R. S., and J. Kaplan. 1986. Intracellular pools of transferrin receptors result from constitutive internalization of unoccupied receptors. Proc Natl Acad Sci USA. 83:6445-9.

Ajmani, P. S., F. Tang, S. Krishnaswami, E. M. Meyer, C. Sumners, and J. A. Hughes. 1999. Enhanced transgene expression in rat brain cell cultures with a disulfide-containing cationic lipid. Neurosci Lett. 277:141-4.

Akhtar, S., S. Basu, E. Wickstrom, and R. L. Juliano. 1991. Interactions of antisense DNA oligonucleotide analogs with phospholipid membranes. Nucleic Acids Res. 19:5551-5559.

Akhtar, S., M. D. Hughes, A. Khan, M. Bibby, M. Hussain, Q. Nawaz, J. Double, and P. Sayyed. 2000. The delivery of antisense therapeutics. Advanced Drug Delivery Reviews. 44:3-21.

Altschul, S. F., T. L. Madden, A. A. Schaffer, J. Zhang, Z. Zhang, W. Miller, and D. J. Lipman. 1997. Gapped BLAST and PSI-BLAST: a new generation of protein database search programs. Nucleic Acids Res. 25:3389-402.

Azzazy, H. M. E., K. Hong, M. -C. Wu, and G. W. Gross. 1995. Interaction of cationic liposomes with cells of electrically active neuronal networks in culture. Brain Res. 695:231-236.

Bacskai, B. J., M. Q. Xia, D. K. Strickland, G. W. Rebeck, and B. T. Hyman. 2000. The endocytic receptor protein LRP also mediates neuronal calcium signaling via N-methyl-D-aspartate receptors. Proc Natl Acad Sci USA. 97:11551-6.

Bally, M. B., P. Harvie, F. M. Wong, S. Kong, E. K. Wasan, and D. L. Reimer. 1999. Biological barriers to cellular delivery of lipid-based DNA carriers. Advanced Drug Delivery Reviews. 38:291-315.

Bandyopadhyay, S., M. Tarek, and M. L. Klein. 1999. Molecular dynamics study of a lipid-DNA complex. Journal of Physical Chemistry B. 103:10075-10080.

Bathori, G., I. Parolini, F. Tombola, I. Szabo, A. Messina, M. Oliva, V. De Pinto, M. Lisanti, M. Sargiacomo, and M. Zoratti. 1999. Porin is present in the plasma membrane where it is concentrated in caveolae and caveolae-related domains. J Biol Chem. 274:29607-12.

Bauerfeind, R., K. Takei, and P. De Camilli. 1997. Amphiphysin I is associated with coated endocytic intermediates and undergoes stimulation-dependent dephosphorylation in nerve terminals. J Biol Chem. 272:30984-92.

Beattie, E. C., C. L. Howe, A. Wilde, F. M. Brodsky, and W. C. Mobley. 2000. NGF signals through TrkA to increase clathrin at the plasma membrane and enhance clathrin-mediated membrane trafficking. J Neurosci. 20:7325-33.

Bell, R. M., and D. J. Burns. 1991. Lipid activation of protein kinase C. J Biol Chem. 266:4661-4.

Bowen, B. J., and E. H. Morgan. 1988. Effect of osmolar and ionic composition of the extracellular fluid on transferrin endocytosis and exocytosis and iron uptake by reticulocytes. J Cell Physiol. 134:1-12.

Brodin, L., P. Low, and 0. Shupliakov. 2000. Sequential steps in clathrin-mediated synaptic vesicle endocytosis. Curr Opin Neurobiol. 10:312-20.

Brosius, B., and D. Riesner. 1986. Influence of lipid membranes on the conformational transitions of nucleic acids. J Biomol Struct Dyn. 4:271-90.

Brown, M. D., G A. Banker, I. M. Hussaini, S. L. Gonias, and S. R. VandenBerg. 1997a. Low density lipoprotein receptor-related protein is expressed early and becomes restricted to a somatodendritic domain during neuronal differentiation in culture. Brain Research. 747:313-317.

Brown, M. S., J. Herz, and J. L. Goldstein. 1997b. LDL-receptor structure. Calcium cages, acid baths and recycling receptors. Nature. 388:629-30.

Bu, G., E. A. Maksymovitch, J. M. Nerbonne, and A. L. Schwartz. 1994. Expression and function of the low density lipoprotein receptor-related protein (LRP) in mammalian central neurons. Journal of Biological Chemistry. 269:18521-18528.

Bu, G., Y. Sun, A. L. Schwartz, and D. M. Holtzman. 1998. Nerve growth factor induces rapid increases in functional cell surface low density lipoprotein receptor-related protein. J Biol Chem. 273:13359-65.

Buchner, K., E. Adamec, M. L. Beermann, and R. A. Nixon. 1999. Isoform-specific translocation of protein kinase C following glutamate administration in primary hippocampal neurons. Brain Res Mo! Brain Res. 64:222-35.

Chen, R. W., and D. M. Chuang. 1999. Long term lithium treatment suppresses p53 and Bax expression but increases Bcl-2 expression. A prominent role in neuroprotection against excitotoxicity. J Biol Chem. 274:6039-42.

Chen, R. W., P. A. Saunders, H. Wei, Z. Li, P. Seth, and D. M. Chuang. 1999. Involvement of glyceraldehyde-3-phosphate dehydrogenase (GAPDH) and p53 in neuronal apoptosis: evidence that GAPDH is upregulated by p53. J Neurosci. 19:9654-62.

Cupers, P., A. Veithen, A. Kiss, P. Baudhuin, and P. J. Courtoy. 1994. Clathrin polymerization is not required for bulk-phase endocytosis in rat fetal fibroblasts. J Cell Biol. 127:725-35.

de Hoop, M. J., and C. G. Dotti. 1993. Membrane traffic in polarized neurons in culture. J Cell Sci Suppl. 17:85-92.

Descamps, O., D. Bilheimer, and J. Herz. 1993. Insulin stimulates receptor-mediated uptake of apoE-enriched lipoproteins and activated alpha 2-macroglobulin in adipocytes. J Biol Chem. 268:974-81.

Dolmer, K., W. Huang, and P. G. Gettins. 2000. NMR solution structure of complement-like repeat CR3 from the low density lipoprotein receptor-related protein. Evidence for specific binding to the receptor binding domain of human alpha(2)-macroglobulin. J Biol Chem. 275:3264-9.

Dubinsky, J. M. 1993. Intracellular calcium levels during the period of delayed excitotoxicity. J Neurosci. 13:623-31.

Dubinsky, J. M., B. S. Kristal, and M. Elizondo-Fournier. 1995. An obligate role for oxygen in the early stages of glutamate-induced, delayed neuronal death. J Neurosci. 15:7071-8.

Fire, E., C M. Brown, M. G. Roth, Y. I. Henis, and N. O. Petersen. 1997. Partitioning of proteins into plasma membrane microdomains. Clustering of mutant influenza virus hemagglutinins into coated pits depends on the strength of the internalization signal. J Biol Chem. 272:29538-45.

FitzGerald, D. J., C. M. Fryling, A Zdanovsky, C. B. Saelinger, M. Kounnas, J. A. Winkles, D. Strickland, and S. Leppla. 1995. Pseudomonas exotoxin mediated selection yields cells with altered expression of low-density lipoprotein receptor-related protein. J Cell Biol. 129:1533-41.

Freedman, S. D., M. H. Katz, E. M. Parker, and A. Gelrud. 1999. Endocytosis at the apical plasma membrane of pancreatic acinar cells is regulated by tyrosine kinases. Am J Physiol. 276:C306-11.

Gaidarov, I., F. Santini, R. A. Warren, and J. H. Keen. 1999. Spatial control of coated-pit dynamics in living cells. Nat Cell Biol. 1: 1-7.

Garcia-Chaumont, C., O. Seksek, J. Grzybowska, E. Borowski, and J. Bolard, 2000. Delivery systems for antisense oligonucleotides. Pharmacol Ther. 87:255-277.

Gonzalez-Zulueta, M., L. M. Ensz, G. Mukhina, R. M. Lebovitz, R. M. Zwacka, J. F. Engelhardt, L. W. Oberley, V. L. Dawson, and T. M. Dawson. 1998. Manganese superoxide dismutase protects nNOS neurons from NMDA and nitric oxide-mediated neurotoxicity. J Neurosci. 18:2040-55.

Goretzki, L., and B. M. Mueller. 1998. Low-density-lipoprotein-receptor-related protein (LRP) interacts with a GTP-binding protein. Biochem J. 336:381-6.

Grzanna, R., J. R. Dubin, G. W. Dent, Z. Ji, W. Zhang, S. P. Ho, and P. R. Hartig. 1998. Intrastriatal and intraventricular injections of oligodeoxynucleotides in the rat brain: tissue penetration, intracellular distribution and c-fos antisense effects. Brain Res Mol Brain Res. 63:35-52.

Hansen, S. H., K. Sandvig, and B. van Deurs. 1993 Clathrin and HA2 adaptors: effects of potassium depletion, hypertonic medium, and cytosol acidification. J Cell Biol. 121:61-72.

Hartmann, G., A. Krug, M. Bidlingmaier, U. Hacker, A. Eigler, R. Albrecht, C. J. Strasburger, and S. Endres. 1998. Spontaneous and cationic lipid-mediated uptake of antisense oligonucleotides in human monocytes and lymphocytes. J Pharmacol Exp Ther. 285:920-8.

Hashimoto, Y., H. Jiang, T. Niikura, Y. Ito, A. Hagiwara, K. Umezawa, V. Abe, V. Murayama, and I. Nishimoto. 2000. Neuronal apoptosis by apolipoprotein E4 through low-density lipoprotein receptor-related protein and heterotrimeric GTPases. J Neurosci. 20:8401-9.

Herz, J., R. C. Kowal, J. L. Goldstein, and M. S. Brown. 1990. Proteolytic processing of the 600 kd low density lipoprotein receptor-related protein (LRP) occurs in a trans-Golgi compartment. Embo J 9:1769-76.

Heuser, J. E., and R. G. Anderson. 1989. Hypertonic media inhibit receptor-mediated endocytosis by blocking clathrin-coated pit formation. J Cell Biol. 108:389-400.

Huang, W., K. Dolmer, and P. G. Gettins. 1999. NMR solution structure of complement-like repeat CR8 from the low density lipoprotein receptor-related protein. J Biol Chem. 274:14130-6.

Hughes, J. A., C. F. Bennett, P. D. Cook, C. J. Guinosso, C. K. Mirabelli, and R. L, Juliano. 1994. Lipid membrane permeability of 2′-modified derivatives of phosphorothioate oligonucteotides. J Pharm Sci. 83:597-600.

Hyman, B. T., D. Strickland, and G. W. Rebeck. 2000. Role of the low-density lipoprotein receptor-related protein in beta-amyloid metabolism and Alzheimer disease. Arch Neurol. 57:646-50.

Jaaskelainen, I., J. Monkkonen, and A. Urtti. 1994. Oligonucleotide-cationic liposome interactions. A physicochemical study. Biochim Biophys Acta. 1195:115-23.

Jimenez, G. S., S. H. Khan, J. M. Stommel, and G. M. WahI. 1999. p53 regulation by post-translational modification and nuclear retention in response to diverse stresses. Oncogene. 18:7656-65.

Jordan, J., M. F. Galindo, J. H. Prehn, R. R. Weichselbaum, M. Beckett, G. D. Ghadge, R. P. Roos, J. M. Leiden, and R. J. Miller. 1997. p53 expression induces apoptosis in hippocampal pyramidal neuron cultures. J Neurosci. 17:1397-405.

Junker, M., and G. E. Creutz. 1993. Endonexin (annexin 1V)-mediated lateral segregation of phosphatidylglycerol in phosphatidylglycerol phosphatidylcholine membranes. Biochemistry. 32:9968-74.

Kaech, S., J. B. Kim, M. Cariola, and E. Ralston. 1996. Improved lipid-mediated gene transfer into primary cultures of hippocampal neurons. Brain Res Mol Brain Res. 35:344-8.

Katz, A., I. Vlodavsky, M. Davies, H. Q. Miao, S. A. Ben-Sasson, D. Darmon, H. Hurwitz, H. Borgel, and M. Benezra. 1997. Antiproliferative activity to glomerular mesangial cells and receptor binding of a heparin-mimicking polyaromatic anionic compound. J Am Soc Nephrol. 8:1688-97.

Kavalali, E. T., J. Klingauf, and R. W. Tsien. 1999. Properties of fast endocytosis at hippocampal synapses. Philos Trans R Soc Lond B Biol Sci. 354:337-46.

Kittler, J. T., P. Delmas, J. N. Jovanovic, D. A. Brown, T. G. Smart, and S. J. Moss. 2000. Constitutive Endocytosis of GABAA Receptors by an Association with the Adaptin AP2 Complex Modulates Inhibitory Synaptic Currents in Hippocampal Neurons. J Neurosci. 20:7972-7977.

Klausner, R D., and A. M. Kleinfeld. 1984. Lipid domains in membranes. In Cell Surface Dynamics: Concepts and Models. A. S. Perelson, C. DeLisi, and F. W. Wiegel, editors. Marcel Dekker, Inc., New York. 23-58.

Klingauf, J., E. T. Kavalali, and R. W. Tsien. 1998. Kinetics and regulation of fast endocytosis at hippocampal synapses. Nature. 394:581-5.

Ko, K. W., R. K. Avramoglu, R. S. McLeod, J. Vukmirica, and Z. Yao. 2001. The Insulin-Stimulated Cell Surface Presentation of Low Density Lipoprotein Receptor-Related Protein in 3T3-L1 Adipocytes Is Sensitive to Phosphatidylinositide 3-Kinase Inhibition. Biochemistry. 40:752-759.

Ko, K. W., R. S. McLeod, R. K. Avramoglu, J. Nimpf, D. J. FitzGerald, J. Vukmirica, and Z. Yao. 1998. Mutation at the processing site of chicken low density lipoprotein receptor-related protein impairs efficient endoplasmic reticulum exit, but proteolytic cleavage is not essential for its endocytic functions. J Biol Chem. 273:27779-85.

Kobayashi, T., F. Gu, and J. Gruenberg. 1998. Lipids, lipid domains and lipid-protein interactions in endocytic membrane traffic. Semin Cell Dev Biol. 9:517-26.

Kobayashi, T., B. Storrie, K. Simons, and C. G. Dotti. 1992. A functional barrier to movement of lipids in polarized neurons. Nature. 359:647-50.

Kodama, M., and T. Miyata. 1996. Effect of the head group of phospholipids on the acyl chain packing and structure of their assemblies as revealed by microcalorimetry and electron microscopy. Colloids and Surfaces A: Physicochemical and Engineering Aspects. 109:283-289.

Komarov, P. G., E. A. Komarova, R. V. Kondratov, K. Christov-Tselkov, J. S. Coon, M. V. Chernov, and A. V. Gudkov. 1999. A chemical inhibitor of p53 that protects mice from the side effects of cancer therapy. Science. 285:1733-1737.

Koppenol, S., F. H. Tsao, H. Yu, and G. Zografi. 1998. The interaction of lung annexin I with phospholipid monolayers at the air/water interface. Biochim Biophys Acta. 1369:221-32.

Kounnas, M. Z., D. A. Chappell, H. Wong, W. S. Argraves, and D. K. Strickland. 1995a. The cellular internalization and degradation of hepatic lipase is mediated by low density lipoprotein receptor-related protein and requires cell surface proteoglycans. J Biol Chem. 270:9307-12.

Kounnas, M Z., R. D. Moir, G. W. Rebeck, A. I. Bush, W. S. Argraves, R. E. Tanzi, B. T. Hyman, and D. K. Strickland. 1995b. LDL receptor-related protein, a multifunctional apoE receptor, binds secreted β-amyloid precursor protein and mediates its degradation. Cell. 82:331-340.

Lai, M. M., H. R. Luo, P. E. Burnett, J. J. Hong, and S. H. Snyder. 2000. The calcineurin-binding protein cain is a negative regulator of synaptic vesicle endocytosis. J Biol Chem. 275: 34017-20.

Lakkaraju, A., J. M. Dubinsky, W. C. Low, and Y. E. Rahman. 2001. Neurons are protected from excitotoxic death by p53 antisense oligonucleotides delivered in anionic liposomes. Manuscript in submission.

Lamaze, C., L. M. Fujimoto, H. L. Yin, and S. L. Schmid. 1997. The actin cytoskeleton is required for receptor-mediated endocytosis in mammalian cells. J Biol Chem. 272:20332-5.

Lee, J. M., G. J. Zipfel, and D. W. Choi. 1999. The changing landscape of ischaemic brain injury mechanisms. Nature. 399:A7-14.

Li, G., C. D'Souza-Schorey, M. A. Barbieri, R. L. Roberts, A. Klippel, L. T. Williams, and P. D. Stahl. 1995. Evidence for phosphatidylinositol 3-kinase as a regulator of endocytosis via activation of Rab5. Proc Natl Acad Sd USA. 92:10207-11.

Liu, Y., M. Jones, C. M. Hingtgen, G. Bu, N. Laribee, R. E. Tanzi, R. D. Moir, A. Nath, and J. J. He. 2000. Uptake of HIV-1 tat protein mediated by low-density lipoprotein receptor-related protein disrupts the neuronal metabolic balance of the receptor ligands. Nat Med. 6:1380-7.

Lorenz, P., T. Misteli, B. F. Baker, C. F. Bennett, and D. L. Spector. 2000. Nucleocytoplasmic shuttling: a novel in vivo property of antisense phosphorothioate ol igodeoxyn ucleotides. Nucleic Acids Res. 28:582-592.

Luo, D., and W. M. Saltzman. 2000. Synthetic DNA delivery systems. Nat Biotechnol. 18:33-37.

MacDonald, R. I. 1990. Characteristics of self-quenching of the fluorescence of lipid-conjugated rhodamine in membranes. J Biol Chem. 265:13533-9.

Malghani, M. S., and J. Yang. 1998. Stable binding of DNA to zwitterionic lipid bilayers in aqueous solutions. Journal of physical Chemistry B. 102:8930-8933.

Man, Y. H., J. W. Lin, W. H. Ju, G. Ahmadian, L. Liu, L. E. Becker, M. Sheng, and Y. T. Wang. 2000. Regulation of AMPA receptor-mediated synaptic transmission by clathrin-dependent receptor internalization. Neuron. 25:649-62.

Maples, C. J., W. G. Ruiz, and G. Apodaca. 1997. Both microtubules and actin filaments are required for efficient postendocytotic traffic of the polymeric immunoglobulin receptor in polarized Madin-Darby canine kidney cells. J Biol Chem. 272:6741-51.

Marchenko, N. D., A. Zaika, and U. M. Moll. 2000. Death signal-induced localization of p53 protein to mitochondria. J Biol Chem. 275:16202-16212.

Marcusson, E. G., B. Bhat, M. Manoharan, C. F. Bennett, and N. M. Dean. 1998. Phosphoroth bate oligodeoxyribonucleotides dissociate from cationic lipids before entering the nucleus. Nucleic Acids Research. 26:2016-2023.

Martin, T. F. 1997. Phosphoinositides as spatial regulators of membrane traffic. Curr Opin Neurobiol. 7:331-8.

Martys, J. L., C. Wjasow, D. M. Gangi, M. C. Kielian, T. E. McGraw, and J. M. Backer. 1996. Wortmannin-sensitive trafficking pathways in chinese hamster ovary cells: Differential effects on endocytosis and lysosomat sorting. Journal of Biological Chemistry. 271:10953-10962.

Mayor, S., S. Sabharanjak, and F. R. Maxfield. 1998. Cholesterol-dependent retention of GPI-anchored proteins in endosomes. Embo J 17:4626-38.

Mikhailenko, I., D. Krylov, K. M. Argraves, D. D. Roberts, G. Liau, and D. K. Strickland. 1997. Cellular internalization and degradation of thrombospondun-1 is mediated by the amino-terminal heparin binding domain (HBD). High affinity interaction of dimeric HBD with the low density lipoprotein receptor-related protein. J Biol Chem. 272:6784-91.

Moestrup, S. K., J. Gliemann, and G. Pallesen. 1992. Distribution of the alpha 2-macroglobulin receptor/low density lipoprotein receptor-related protein in human tissues. Cell Tissue Res. 269:375-82.

Monnard, P. A., N. Berclaz, K. Conde-Frieboes, and T. Oberholzer. 1999. Decreased solute entrapment in 1-palmitoyl-2-oleoyl-sn-glycero-3-phosphocholine liposomes prepared by freeze/thaw in the presence of physiological amounts of monovalent salts. Langmuir. 15:7504-7509.

Morrison, R. S., and Y. Kinoshita. 2000. The role of p53 in neuronal cell death. Cell Death Differ. 7:868-879.

Morrison, R. S., H. J. Wenzel, Y. Kinoshita, C. A. Robbins, L. A. Donehower, and P. A. Schwartzkroin. 1996. Loss of the p53 tumor suppressor gene protects neurons from kainate-induced cell death. J Neurosci. 16:1337-45.

Mukherjee, S., T. T. Soe, and F. R. Maxfield. 1999. Endocytic sorting of lipid analogues differing solely in the chemistry of their hydrophobic tails. J Cell-Biol. 144:1271-84.

Narita, M., G. Bu, G. M. Olins, D. A Higuchi, J. Herz, G. J. Broze, and A. L. Schwartz. 1995. Two receptor systems are involved in the plasma clearance of tissue factor pathway inhibitor in vivo. J Biol Chem. 270:24800-4.

Narita, M., D. M. Holtzman, A. L. Schwartz, and G. Bu. 1997. Alpha2-macroglobulin complexes with and mediates the endocytosis of beta-amyloid peptide via cell surface low-density lipoprotein receptor-related protein. J Neurochem. 69:1904-11.

Oka, J. A., M. D. Christensen, and P. H. Weigel. 1989. Hyperosmolarity inhibits galactosyl receptor-mediated but not fluid phase endocytosis in isolated rat hepatocytes. J Biol Chem. 264:12016-24.

Pfeffer, S. R. 1999. Motivating endosome motility. Nat Cell Biolog. 1:E145-E 147.

Polyak, K., Y. Xia, J. L. Zweier, K. W. Kinzler, and B. Vogelstein. 1997. A model for p53-induced apoptosis. Nature. 389:300-5.

Powell, K. A., V. A. Valova, C. S. Malladi, O. N. Jensen, M. R. Larsen, and P. J. Robinson. 2000. Phosphorylation of dynamin I on Ser-795 by protein kinase C blocks its association with phospholipids. J Biol Chem. 275:11610-7.

Prekeris, R., D. L. Foletti, and R. H. Scheller. 1999. Dynamics of tubulovesicular recycling endosomes in hippocampal neurons. J Neurosci. 19:10324-37.

Rodenburg, K. W., L. Kjoller, N. H. Petersen, and P. A. Andreasen. 1998. Binding of urokinase-type plasminogen activator-plasminogen activator inhibitor-I complex to the endocytosis receptors alpha2-macroglobulin receptor/low-density lipoproteun receptor-related protein and very-low-density lipoprotein receptor involves basic residues in the inhibitor. Biochem J. 329: 55-63.

Sattler, R., Z. Xiong, WY. Lu, M. Hafner, J. F. MacDonald, and M. Tymianski. 1999. Specific coupling of NMDA receptor activation to nitric oxide neurotoxicity by PSD-95 protein. Science. 284:1845-8.

Schwartz, B., C. Benoist, B. Abdallah, D. Scherman, J. P. Behr, and B. A. Demeneix. 1995. Lipospermine-based gene transfer into the newborn mouse brain is optimized by a low lipospermine/DNA charge ratio. Hum Gene Ther. 6:1515-24.

Shpetner, H., M. Joly, D. Hartley, and S. Corvera. 1996. Potential sites of P1-3 kinase function in the endocytic pathway revealed by the P1-3 kinase inhibitor, wortmannin. J Cell Biol. 132:595-605.

Shuptiakov, 0, P. Low, D. Grabs, H. Gad, H. Chen, C. David, K. Takei, P. De Camilli, and L. Brodin. 1997. Synaptic vesicle endocytosis impaired by disruption of dynamin-SH3 domain interactions. Science. 276:259-63.

Singer, V. L., L. J. Jones, and S. Yue. 1995. Highly sensitive fluorescence assay for the detection and quantitation of oligonucleotides in complex mixtures. FASEB J 9:A1422.

Sofer, A., and A. H. Futerman. 1996. Rate of retrograde transport of cholera toxin from the plasma membrane to the Golgi apparatus and endoplasmic reticulum decreases during neuronal development. J Neurochem. 67:2134-40.

Son, K. K., D. H. Patel, D. Tkach, and A. Park. 2000. Cationic liposome and plasmid DNA complexes formed in serum-free medium under optimum transfection condition are negatively charged. Biochim Biophys Acta. 1466:11-15.

Sonnichsen, B., S. De Renzis, E. Nielsen, J. Rietdorf, and M. Zeria!. 2000. Distinct membrane domains on endosomes in the recycling pathway visualized by multicolor imaging of Rab4, Rab5, and Rab11. J Cell Biol. 149:901-14.

Soussi, T., C. Caron de Fromentel, C. Breugnot, and E. May. 1988. Nucleotide sequence of a cDNA encoding the rat p53 nuclear oncoprotein. Nucleic Acids Res. 16:11384.

Stein, C. A. 1999. Keeping the biotechnology of antisense in context. Nat Biotechnol. 17:209.

Stein, C. A. 1999. Two problems in antisense biotechnology: in vitro delivery and the design of antisense experiments. Biochim Biophys Acta. 1489:45-52.

Stewart, J. C. 1980. Colorimetric determination of phospholipids with ammonium ferrothiocyanate. Anal Biochem. 104:10-4.

Struck, D. K., D. Hoekstra, and R. E. Pagano. 1981. Use of resonance energy transfer to monitor membrane fusion. Biochemistry. 20:4093-9.

Swaiman, K. F., and V. L. Machen. 1986. Transferrin binding by mammalian cortical cells. Neurochem Res. 11:1241-8.

Sweitzer, S. M., and J. E. Hinshaw. 1998. Dynamin undergoes a GTP-dependent conformational change causing vesiculation. Cell. 93:1021-9.

Szabo, I., G. Bathori, F. Tombola, A. Coppola, I. Schmehl, M. Brini, A. Ghazi, V. De Pinto, and M. Zoratti. 1998. Double-stranded DNA can be translocated across a planar membrane containing purified mitochondrial porin. Faseb J 12:495-502.

Takenaka, I., F. Morin, B. R. Seizinger, and N. Kley. 1995. Regulation of the sequence-specific DNA binding function of p53 by protein kinase C and protein phosphatases. J Bid Chem. 270:5405-11.

Tolar, M., M. A. Marques, J. A. Harmony, and K. A. Crutcher. 1997. Neurotoxicity of the 22 kDa thrombin-cleavage fragment ofapolipoprotein E and related synthetic peptides is receptor-mediated. J Neurosci. 17:5678-86.

Trommsdorff, M., J. P. Borg, B. Margolis, and J. Herz. 1998. Interaction of cytosolic adaptor proteins with neuronal apolipoprotein E receptors and the amyloid precursor protein. J Biol Chem. 273:33556-60.

Walicke, P. A., and A. Baird. 1991. Internalization and processing of basic fibroblast growth factor by neurons and astrocytes. J Neurosci. 11:2249-58.

Wangerek, L. A., H. -H. M. Dahl, T. J. Senden, J. B. Carlin, D. A. Jans, D. E. Dustan, P. A. Ioannou, R. Williamson, and S. M. Forrest. 2001. Atomic force microscopy imaging of DNA-cationic liposome complexes optimised for gene transfection into neuronal cells. Journal of Gene Medicine. 3:72-81.

Warshawsky, I., J. Herz, G. J. Broze, and A. L. Schwartz. 1996. The low density lipoprotein receptor-related protein can function independently from heparan sulfate proteoglycans in tissue factor pathway inhibitor endocytosis. J Biol Chem. 271:25873-9.

Wigge, P., and H. T. McMahon. 1998. The aniphiphysin family of proteins and their role in endocytosis at the synapse. Trends in Neurological Sciences. 21:339-344.

Willem, J., M. ter Beest, G. Scherphof, and D. Hoekstra. 1990. A non-exchangeable fluorescent phospholipid analog as a membrane traffic marker of the endocytic pathway. Eur J Cell Biol. 53:173-84.

Williams, S. E., J. D. Ashcom, W. S. Argraves, and D. K. Strickland. 1992. A novel mechanism for controlling the activity of alpha 2-macroglobulin receptor/low density lipoprotein receptor-related protein. Multiple regulatory sites for 39-kDa receptor-associated protein. J Biol Chem. 267:9035-40.

Willnow, T. E. 1999. The low-density lipoprotein receptor gene family: multiple roles in lipid metabolism. J Mol Med. 77:306-15.

Willnow, T. E., A. Nykjaer, and J. Herz. 1999. Lipoprotein receptors: new roles for ancient proteins. Nat Cell Biol. 1 μl 57-62.

Willnow, T. E., K. Orth, and J. Herz. 1994a. Molecular dissection of ligand binding sites on the low density lipoprotein receptor-related protein. J Biol Chem. 269:15827-32.

Willnow, T. E., Z. Sheng, S. Ishibashi, and J. Herz. 1994b. Inhibition of hepatic chylomicron remnant uptake by gene transfer of a receptor antagonist. Science. 264:1471-4.

Xiang, H., D. W. Hochman, H. Saya, T. Fujiwara, P. A. Schwartzkroin, and R. S. Morrison. 1996. Evidence for p53-mediated modulation of neuronal viability. Journal of Neuroscience. 16:6753-65.

Xu, Y., S. W. Hui, P. Frederik, and F. C. Szoka, Jr. 1999. Physicochemical characterization and purification of cationic lipoplexes. Biophys J. 77: 341-53.

Zabner, J., A. J. Fasbender, T. Moninger, K. A. Poellinger, and M. J. Welsh. 1995. Cellular and molecular barriers to gene transfer by a cationic lipid. J Biol Chem. 270:18997-9007.

Zakharenko, S., and S. Popov. 1998. Dynamics of axonal microtubules regulate the topology of new membrane insertion into the growing neurites. J Cell Biol. 143:1077-86.

Zelphati, O., and J. F. C. Szoka. 1996. Intracellular distribution and mechanism of delivery of oligonucleotides mediated by cationic lipids. Pharmaceutical Research. 13:1367-1372.

Zelphati, 0., and F. C. Szoka. 1996. Liposomes as a carrier for intracellular delivery of oligonucleotides: a real or magic bullet? J Controlled Release 41:99-119.

Zhang, Y. P., R. N. Lewis, and R. N. McElhaney. 1997. Calorimetric and spectroscopic studies of the thermotropic phase behavior of the n-saturated 1,2-diacylphosphatidyl glycerols. Biophys J. 72:779-93.

Zhuo, M., D. M. Holtzman, Y. Li, H. Osaka, J. DeMaro, M. Jacquin, and G. Bu. 2000. Role of tissue plasminogen activator receptor LRP in hippocampal long-term potentiation. J Neurosci. 20:542-9.

All references cited herein are hereby incorporated by reference.

What is claimed is:

1. A pharmaceutical composition comprising:
(a) an anionic liposome comprising a phospholipid with a head group selected from the group consisting of sn-glycero-phosphocholine; sn-glycero-phospho-rac-(1-glycerol); sn-glycero-phospho-L-serine; sn-glycero-3-phosphate; and combinations thereof;
(b) a bioactive agent; and
(c) a cation, a buffer, or a combination thereof;
wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

2. The pharmaceutical composition of claim 1 wherein the anionic liposome is 1,2-dioleoyl-sn-glycero-3-phosphocholine (DOPC); 1 ,2-dioleoyl-sn-glycero-3-[phospho-rac-(1-gylcerol)] (DOPG); 1,2-dioleoyl-sn-glycero-3-[phospho-L-serine] (DOPS); 1,2-dioleoyl-sn-glycero-3-phosphate (DOPA); or a combination thereof.

3. The pharmaceutical composition of claim 1 wherein the anionic liposome is DOPC/DOPG.

4. The pharmaceutical composition of claim 1 wherein the anionic liposome is DOPC/DOPG wherein the ratio of DOPC to DOPG is about 88:12.

5. The pharmaceutical composition of claim 1 wherein the mean diameter of the anionic liposome is about 20 nm to about 5 microns.

6. The pharmaceutical composition of claim 1 wherein the mean diameter of the anionic liposome is about 75 nm to about 500 nm.

7. The pharmaceutical composition of claim 1 wherein the mean diameter of the anionic liposome is about 175 nm to about 225 nm.

8. The pharmaceutical composition of claim 1 wherein the anionic liposome is present up to about 500 mM in the pharmaceutical composition.

9. The pharmaceutical composition of claim 1 wherein the anionic liposome is present in about 2.5 mM to about 30 mM in the pharmaceutical composition.

10. The pharmaceutical composition of claim 1 wherein the bioactive agent has a molecular weight of about 250 to about 750 and the anionic liposome is present in about 5 mM to about 25 mM in the pharmaceutical composition.

11. The pharmaceutical composition of claim 1 wherein the bioactive agent has a molecular weight of about 750 to about 1500 and the anionic liposome is present in about 7.5 mM to about 25 mM in the pharmaceutical composition.

12. The pharmaceutical composition of claim 1 wherein the bioactive agent is an oligonucleotide having a length of about 5 bases to about 50 bases and the anionic liposome is present in about 2.5 mM to about 25 mM in the pharmaceutical composition.

13. The pharmaceutical composition of claim 1 wherein the bioactive agent is an oligonucleotide having a length of about 15 bases to about 30 bases and the anionic liposome is present in about 2.5 mM to about 25 mM in the pharmaceutical composition.

14. The pharmaceutical composition of claim 1 wherein the bioactive agent is a protein having a molecular weight of up to about 75,000 and the anionic liposome is present in about 2.5 mM to about 30 mM in the pharmaceutical composition.

15. The pharmaceutical composition of claim 1 wherein the bioactive agent is a protein having a molecular weight of about 1,000 to about 5,000 and the anionic liposome is present in about 2.5 mM to about 30 mM in the pharmaceutical composition.

16. The pharmaceutical composition of claim 1 wherein the bioactive agent is double- or single-stranded genetic material, or a fragment thereof, having a molecular weight of up to about 1×10⁸ and the anionic liposome is present in about 2.5 mM to about 40 mM in the pharmaceutical composition.

17. The pharmaceutical composition of claim 1 wherein the bioactive agent is double- or single-stranded genetic material, or a fragment thereof, having a molecular weight of about 1×10⁵ to about 1×10⁷ and the anionic liposome is present in about 2.5 mM to about 40 mM in the pharmaceutical composition.

18. The pharmaceutical composition of claim 1 wherein the bioactive agent is an antiviral agent; an antibacterial agent; an antifungal agent; an antineoplastic agent; an anti-inflammatory agent; a radiolabel; a peptide; a protein; an oligonucleotide; a hormone; a carbohydrate; a growth factor; a cytokine; a radioopaque compound; a fluorescent compound; a mydriatic compound; a bronchodilator; a local anesthetic; a nucleic acid sequence; double or single stranded genetic material, or a fragment thereof; an analgesic; an antiparasitic; an antipsychotic; an antispasmodic; an arthritis medication; a biological; a bone metabolism regulator; a calcium channel blocker; a cardiovascular agent; a central nervous system stimulant; a diabetes agent; a diagnostic; a fungal medication; a gastrointestinal agent; a histamine receptor antagonist; an immunosuppressive; a muscle relaxant; a nausea medication; a nucleoside analogue; a parkinsonism drug; a platelet inhibitor; a psychotropic; a respiratory drug; a sedative; a urinary anti-infective; a urinary tract agent; a vitamin; a nucleotide; a signaling molecule; a fluorescent molecule; a bioactive lipid; a neuroactive agent; an energy substrate; or a combination thereof.

19. The pharmaceutical composition of claim 1 wherein the bioactive agent is double- or single-stranded genetic material, or a fragment thereof.

20. The pharmaceutical composition of claim 1 wherein the bioactive agent is a p53 antisense oligonucleotide.

21. The pharmaceutical composition of claim 1 wherein the bioactive agent is an antisense oligonucleotide having a sequence of SEQ ID NO:1 targeted to human p53 mRNA or an antisense oligonucleotide having a sequence of SEQ ID NO:2 targeted to rat p53 mRNA.

22. The pharmaceutical composition of claim 1 wherein the bioactive agent is present up to about 50 mM in the pharmaceutical composition.

23. The pharmaceutical composition of claim 1 wherein the bioactive agent is present in about 1 femtoM to about 1 M in the pharmaceutical composition.

24. The pharmaceutical composition of claim 1 wherein the bioactive agent is present in about 2 nM to about 10 mM in the pharmaceutical composition.

25. The pharmaceutical composition of claim 1 wherein the bioactive agent has a molecular weight of about 250 to about 750 and is present in about 0.5 MM to about 10 mM in the pharmaceutical composition.

26. The pharmaceutical composition of claim 1 wherein the bioactive agent has a molecular weight of about 750 to about 1500 and is present in about 0.5 mM to about 5 nM in the pharmaceutical composition.

27. The pharmaceutical composition of claim 1 wherein the bioactive agent is an oligonucleotide having a length of about 5 bases to about 50 bases and is present in about 25 μM to about 250 μM in the pharmaceutical composition.

28. The pharmaceutical composition of claim 1 wherein the bioactive agent is an oligonucleotide having a length of about 15 bases to about 30 bases and is present in about 25 μM to about 250 μM in the pharmaceutical composition.

29. The pharmaceutical composition of claim 1 wherein the bioactive agent is a protein having a molecular weight of up to about 75,000 and is present in about 25 μM to about 250 μM in the pharmaceutical composition.

30. The pharmaceutical composition of claim 1 wherein the bioactive agent is a protein having a molecular weight of about 1,000 to about 5,000 and is present in about 25 μM to about 250 μM in the pharmaceutical composition.

31. The pharmaceutical composition of claim 1 wherein the bioactive agent is double or single stranded genetic material, or a fragment thereof, having a molecular weight of up to about 1×10⁸ and is present in about 2 nM to about 40 nM in the pharmaceutical composition.

32. The pharmaceutical composition of claim 1 wherein the bioactive agent is double or single stranded genetic material, or a fragment thereof, having a molecular weight of about 1×10⁵ to about 1×10⁷ and is present in about 2 nM to about 40 nM in the pharmaceutical composition.

33. The pharmaceutical composition of claim 1 wherein up to about 100% of the bioactive agent is encapsulated in the anionic liposome.

34. The pharmaceutical composition of claim 1 wherein more than about 10% of the bioactive agent is encapsulated in the anionic liposome.

35. The pharmaceutical composition of claim 1 wherein more than about 20% of the bioactive agent is encapsulated in the anionic liposome.

36. The pharmaceutical composition of claim 1 wherein about 55% to about 60% of the bioactive agent is encapsulated in the anionic liposome.

37. The pharmaceutical composition of claim 1 wherein the cation is a monovalent cation.

38. The pharmaceutical composition of claim 1 wherein the cation is Na⁺, K⁺, Li⁺, Fr⁺, Rb⁺, or Cs⁺.

39. The pharmaceutical composition of claim 1 wherein the cation is Na⁺, K⁺, or Li⁺.

40. The pharmaceutical composition of claim 1 wherein the cation is present up to about 50 mM in the pharmaceutical composition.

41. The pharmaceutical composition of claim 1 wherein the cation is present up to about 5 mM in the pharmaceutical composition.

42. The pharmaceutical composition of claim 1 wherein the buffer maintains the pH of the pharmaceutical composition between about 6.0 to about 8.0.

43. The pharmaceutical composition of claim 1 wherein the buffer maintains the pH of the pharmaceutical composition between about 7.0 to about 7.5.

44. The pharmaceutical composition of claim 1 wherein the buffer is HEPES; BES; HEPPS; imidazole; MOPS; TES; TEA; monobasic or dibasic potassium phosphate; monobasic or dibasic sodium phosphate; cacodylic acid; MES; PIPES; glycine amide; glycylglycine; TAPS; boric acid; BIS-TRIS PROPANE; DIPSO; TAPSO; HEPPSO; POPSO; EPPS; TRICINE; BICINE; TAPS; a pharmaceutically acceptable salt thereof; or a combination thereof.

45. The pharmaceutical composition of claim 1 wherein the buffer is [4-(2-hydroxyethyl)-1-piperazine ethanesulfonic acid] (HEPES).

46. The pharmaceutical composition of claim 1 wherein the buffer is present up to about 50 mM in the pharmaceutical composition.

47. The pharmaceutical composition of claim 1 wherein the buffer is present up to about 10 mM in the pharmaceutical composition.

48. The pharmaceutical composition of claim 1 wherein the molar ratio of bioactive agent to anionic liposome is about 10:1 to about 1:1×10¹⁰.

49. The pharmaceutical composition of claim 1 wherein the molar ratio of bioactive agent to anionic liposome is about 5:1 to about 1:10,000.

50. A pharmaceutical composition comprising:
(a) an anionic liposome comprising a phospholipid with a head group selected from the group consisting of sn-glycero-phosphocholine; sn-glycero-phospho-rac-(1-glycerol); sn-glycero-3-phosphate; and combinations thereof;
(b) a bioactive agent; and
(c) a cation, a buffer, or a combination thereof.

51. A pharmaceutical composition comprising:
(a) an anionic liposome;
(b) a bioactive agent is an antisense oligonucleotide having a sequence of SEQ ID NO:1 targeted to human p53 mRNA or an antisense oligonucleotide having a sequence of SEQ ID NO:2 targeted to rat p53 mRNA; and
(c) a cation, a buffer, or a combination thereof.

52. A method of delivering a bioactive agent to a target comprising contacting the target with a composition, wherein the composition comprises:
(a) an anionic liposome comprising a phospholipid with a head group selected from the group consisting of sn-glycero-phosphocholine; sn-glycero-phospho-rac-(1-glycerol); sn-glycero-phospho-L-serine; sn-glycero-3-phosphate; and combinations thereof;
(b) a bioactive agent; and
(c) a cation, a buffer, or a combination thereof;
wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

53. The method of claim 52 wherein the target is a cell of an organ.

54. The method of claim 52 wherein the target is a cell of: the brain, central nervous system, peripheral nervous systems, liver, lung, larynx, bone marrow, spleen, kidney, lymphatic system, hematopoetic system, gastric mucosa, small intestine, large intestine, gall bladder, pancreas, salivary gland, testes, ovary, cervix, uterus, muscle, skin, thyroid gland, parathyroid gland, adrenal gland, connective tissue, chondroid tissue, blood vessel, macrophage, pleura, placenta, a tumor, or a growth.

55. The method of claim 52 wherein the target is non-dividing cells.

56. The method of claim 52 wherein the target is neuronal cells.

57. The method of claim 52 wherein the target is hippocampal neuronal cells.

58. The method of claim 52 wherein the target is a cell that expresses a receptor belonging to the low-density lipoprotein (LDL) gene family.

59. The method of claim 52 wherein the target is a cell that possess a low-density lipoprotein receptor-related protein (LRP) receptor.

60. The method of claim 52 wherein the target is a cell that possesses an endocytic low-density lipoprotein receptor-related protein receptor.

61. The method of claim 52 wherein the target is a cell that possesses a receptor that is expressed in mammalian central nervous system (CNS).

62. The method of claim 52 wherein the target is a pleuripotent cell.

63. The method of claim 62 wherein the target is a stem cell.

64. A method of delivering a bioactive agent to a target comprising contacting the target with a composition, wherein the composition comprises:
(a) an anionic liposome comprising a phospholipid with a head group selected from the group consisting of sn-glycero-phosphocholine; sn-glycero-phospho-rac-(1-glycerol); sn-glycero-3-phosphate; and combinations thereof;
(b) a bioactive agent; and
(c) a cation, a buffer, or a combination thereof.

65. A method of delivering a bioactive agent to a target comprising contacting the target with a composition, wherein the composition comprises:
(a) an anionic liposome;
(b) an antisense oligonucleotide having a sequence of SEQ ID NO:1 targeted to human p53 mRNA or an antisense oligonucleotide having a sequence of SEQ ID NO:2 targeted to rat p53 mRNA; and
(c) a cation, a buffer, or a combination thereof.

66. A method of delivering a bioactive agent to non dividing cells comprising contacting the non dividing cells with a composition, wherein the composition comprises:
(a) an anionic liposome;
(b) a bioactive agent; and
(c) a cation, a buffer, or a combination thereof.


SEQUENCE LISTING (4 sequences)

SEQ ID NO: 1 - DNA, 18 bp, Homo sapiens
ctcgacgctaggatctga

SEQ ID NO: 2 - DNA, 18 bp, Rattus norvegicus
ctgtgaatcctccatgac

SEQ ID NO: 3 - DNA, 18 bp, Artificial Sequence
  An oligonucleotide with a scrambled sequence used as a negative control.
tcgatctacgactgactc

SEQ ID NO: 4 - DNA, 18 bp, Artificial Sequence
  An oligonucleotide with a mismatched sequence used as a negative control.
gagtgaatgatccatggg